(19) United States
(12) Patent Application Publication
(10) Pub. No.: US 2024/0124568 A1
(43) Pub. Date: Apr. 18, 2024

(54) CHEMERIN INHIBITORS AND USES THEREOF FOR TREATING KIDNEY CANCER

(71) Applicant: University of Miami, Miami, FL (US)

(72) Inventor: Scott Welford, Miami, FL (US)

(21) Appl. No.: 18/187,960

(22) Filed: Mar. 22, 2023

Related U.S. Application Data
(60) Provisional application No. 63/322,368, filed on Mar. 22, 2022.

(51) Int. Cl. C07K 16/24; A61K 31/7105; A61P 35/00; C12N 9/22; C12N 15/11

(52) U.S. Cl. CPC C07K 16/24; A61K 31/7105; A61P 35/00; C12N 9/22; C12N 15/11; A61K 2039/505

(57) ABSTRACT

The present disclosure relates to chemerin inhibitors and uses thereof for treating, preventing, and detecting cancer.

### CROSS REFERENCE TO RELATED APPLICATIONS

This application claims the benefit of U.S. Provisional Patent Application Ser. No. 63/322,368 filed Mar. 22, 2022, the disclosure of which is expressly incorporated herein by reference in its entirety.

### REFERENCE TO A SEQUENCE LISTING

The Sequence Listing submitted Mar. 22, 2023 as an XML file named “11348-021US1.xml,” created on Mar. 21, 2023, and having a size of 77,824 bytes is hereby incorporated by reference pursuant to 37 C.F.R. § 1.831 through 1.835.

### FIELD

The present disclosure relates to chemerin inhibitors and uses thereof for treating and/or preventing kidney cancer (e.g., clear cell renal cell carcinoma).

### BACKGROUND

Renal cell carcinoma (RCC) is one of the top ten cancers diagnosed globally, with over 200,000 new cases worldwide each year. RCC currently results in approximately 74,000 cancer cases and 15,000 mortalities annually in the United States; and according to the SEER (Surveillance, Epidemiology, and End Results) database, 16% of RCC patients have distant metastatic disease, with a 5-year survival rate of only 11.6%. The most common subtype of renal cancer is clear cell renal cell carcinoma (ccRCC), which accounts for more than 70% of all kidney cancers. What is needed are novel compositions and methods for preventing and treating kidney cancer (e.g., clear cell renal cell carcinoma).

### SUMMARY

The present disclosure shows that chemerin is overexpressed in subjects having clear cell renal cell carcinoma and further shows that inhibition of chemerin can suppress tumor growth.

Accordingly, in some aspects, disclosed herein is a method of treating kidney cancer (e.g., clear cell renal cell carcinoma) in a subject, comprising administering to the subject a therapeutically effective amount of an inhibitor of chemerin.

In some embodiments, the inhibitor of chemerin is a polypeptide, a polynucleotide, a small molecule, or a gene editing tool.

In some embodiments, the polypeptide is a recombinant antibody. In some embodiments, the recombinant antibody is a humanized antibody.

In some embodiments, the recombinant antibody comprises a heavy chain variable domain (VH) comprising a heavy chain complementarity-determining region (CDRH)1, a CDRH2, and/or a CDRH3, wherein
the CDRH1 comprises a sequence at least 80% identical to SEQ ID NO: 8 or a fragment thereof,
the CDRH2 comprises a sequence at least 80% identical to SEQ ID NO: 9 or a fragment thereof, and
the CDRH3 comprises a sequence at least 80% identical to SEQ ID NO: 10 or a fragment thereof.

In some embodiments, the VH comprises the sequence of SEQ ID NO: 3 or a fragment thereof.

In some embodiments, the recombinant antibody of any preceding aspect comprises a light chain variable domain (VL) comprising a light chain complementarity-determining region (CDRL)1, a CDRL2, and/or a CDRL3, wherein
the CDRL1 comprises a sequence at least 80% identical to SEQ ID NO: 14 or a fragment thereof,
the CDRL2 comprises a sequence at least 80% identical to SEQ ID NO: 15 or a fragment thereof, and
the CDRL3 comprises a sequence at least 80% identical to SEQ ID NO: 16 or a fragment thereof.

In some embodiments, the VL comprises the sequence of SEQ ID NO: 4 or a fragment thereof.

In some embodiments, the polynucleotide is an siRNA or an shRNA. In some embodiments, the gene editing tool is a CRISPR/Cas endonuclease (Cas)9 system. In some embodiments, the CRISPR/Cas9 system comprises a guide RNA comprising a sequence selected from the group consisting of SEQ ID NOs: 17-40 or a fragment thereof.

Also disclosed herein is a method of treating kidney cancer (e.g., clear cell renal cell carcinoma) in a subject, wherein said method comprises:
obtaining a biological sample from the subject;
determining if the biological sample has an increased level of chemerin relative to a reference control; and
administering to the subject a therapeutically effective amount of an anti-cancer agent if the biological sample has an increased level of chemerin.

Also disclosed herein is a method of diagnosing kidney cancer (e.g., clear cell renal cell carcinoma) in a subject, wherein said method comprises:
obtaining a biological sample from the subject;
measuring the level of chemerin in the biological sample; and
determining that the subject has kidney cancer if the biological sample has an increased level of chemerin relative to a reference control.

Also disclosed herein is a recombinant antibody comprising a heavy chain variable domain (VH) comprising a heavy chain complementarity-determining region (CDRH)1, a CDRH2, and/or a CDRH3, wherein
the CDRH1 comprises a sequence at least 80% identical to SEQ ID NO: 8 or a fragment thereof,
the CDRH2 comprises a sequence at least 80% identical to SEQ ID NO: 9 or a fragment thereof, and
the CDRH3 comprises a sequence at least 80% identical to SEQ ID NO: 10 or a fragment thereof.

In some embodiments, the recombinant antibody further comprises a light chain variable domain (VL) comprising a light chain complementarity-determining region (CDRL)1, a CDRL2, and/or a CDRL3, wherein
the CDRL1 comprises a sequence at least 80% identical to SEQ ID NO: 14 or a fragment thereof,
the CDRL2 comprises a sequence at least 80% identical to SEQ ID NO: 15 or a fragment thereof, and
the CDRL3 comprises a sequence at least 80% identical to SEQ ID NO: 16 or a fragment thereof.

Also disclosed herein is a recombinant polynucleotide comprising a nucleic acid sequence encoding the recombinant antibody of any preceding aspect.

Also disclosed herein is an expression vector comprising the recombinant polynucleotide of any preceding aspect.

### DESCRIPTION OF DRAWINGS

The accompanying figures, which are incorporated in and constitute a part of this specification, illustrate aspects described below.

FIGS. 1A-1J show that chemerin is clinically relevant in ccRCC. FIG. 1A shows volcano plots showing the differentially expressed genes from comparison of the ccRCC tumor cluster and normal kidney epithelium cluster from single-cell RNA-sequencing (scRNA-seq). Adipogenic trans-differentiation of normal kidney epithelium to ccRCC is demonstrated by an enrichment of adipogenic genes (red circles, left), downregulation of developmental genes (blue circles, middle) and epithelial-to-mesenchymal (EMT) transition genes (downregulated epithelial genes and upregulated mesenchymal genes; orange circles, right). FIG. 1B shows list of adipkines identified by in silico data mining from The Cancer Genome Atlas (TCGA) and Oncomine databases. FIG. 1C shows scRNA-seq data from human ccRCC specimens presented as a UMAP plot demonstrating overexpression of chemerin in tumor cell cluster. FIG. 1D shows mRNA expression transcripts per million (TPM) of chemerin in ccRCC tumor samples (n=533) and the normal adjacent tissues (n=72) from TCGA. Mann-Whitney U-test. Boxplot represents median and 25th and 75th percentiles, whiskers 1.5 times the interquartile range. FIG. 1E shows mRNA expression TPM of chemerin in Stage I (n=267), Stage II (n=57), Stage III (n=123), Stage IV (n=84) of ccRCC and normal adjacent tissues (n=72) from TCGA. Mann-Whitney U-test. Boxplot represents median and 25th and 75th percentiles, whiskers 1.5 times the interquartile range. FIG. 1F shows Kaplan-Meier curve showing correlation of higher mRNA expression of RARRES2 with poorer survival of ccRCC patients in the TCGA KIRC dataset. Log rank analysis. FIG. 1G shows ELISA analysis of plasma chemerin protein level in 24 normal individuals and 59 ccRCC patients. Mann-Whitney U-test. FIG. 1H shows plasma chemerin protein level stratified according to BMI status. Normal BMI is defined as value <25 kg/m², overweight as BMI 25 kg/m²≤BMI<30 kg/m², and obese as BMI≥30 kg/m². Mann-Whitney U-test. FIG. 1I shows correlation of 20 plasma chemerin expression with corresponding Oil Red O (ORO) staining of tumor sections. Two-tailed student's t-test. FIG. 1J shows correlation of plasma chemerin expression with patient characteristics and tumor pathology. p-values based on χ² analysis. Error bars represent SEM of three independent experiments and three technical replica per experiment. *p<0.05, **p<0.01, ***p<0.001; ****p<0.0001.

FIGS. 2A-2I show that chemerin regulates ccRCC tumor growth in vitro and in vivo. FIG. 2A shows immunoblot of lysate confirming knockdown of chemerin (upper panel) and cell proliferation assay from 786-O cells infected with shGFP control or 3 different shRNAs encoding lentivirus targeting chemerin (shRARRES2-1, shRARRES2-2, shRARRES2-3). Two-way repeated measures ANOVA with Geisser-Greenhouse correction. (FIG. 2B) Same as above for 769-P cells. (FIG. 2C) Same as above for UOK101 cells. (FIG. 2D) Same as above for A-498 cells. (FIG. 2E) Cell proliferation assay of 786-O cells infected with lentivirus encoding either sgCONT or sgRARRES2 (sgRARRES2_1 and sgRARRES2_2). 2 different clones for sgRARRES2 were selected. Two-way repeated measures ANOVA with Geisser-Greenhouse correction. (FIG. 2F) Subcutaneous tumor volume measurement in nude mice implanted with 786-O cells which were infected with lentivirus encoding either shGFP (n=7) or 3 different shRARRES2 (n=7 in each arm). Two-way measures ANOVA with Dunnett's multiple comparison test correction. (FIG. 2G) Same as (FIG. 2F) from 786-O cells infected with lentivirus encoding either sgCONT (n=5) or 2 different sgRARRES2 (n=5 in each arm). Two-way measures ANOVA with Dunnett's multiple comparison test correction. (FIG. 2H) Representative EdU staining and quantification of 786-O and 769-P cells infected with shGFP control or 2 different shRARRES2 (shRARRES2-1, shRARRES2-2). One-way ANOVA. (FIG. 2I) Flow cytometry for annexin V and propidium iodide staining of 786-O and 769-P cells infected with shGFP control or 2 different shRARRES2 (shRARRES2-1, shRARRES2-2). One-way ANOVA. Error bars represent SEM of three independent experiments and three technical replica per experiment. *p<0.05, **p<0.01, ***p<0.001, ****p<0.0001.

FIGS. 3A-3G show that chemerin suppresses fatty acid oxidation. (FIG. 3A) BODIPY staining of 786-O cells infected with lentivirus targeting shGFP control or 2 different shRARRES2 (upper panels). DAPI staining of nucleus in lower panels. Quantification of BODIPY staining, normalized to DAPI staining, in 786-O cells infected with lentivirus targeting shGFP control or 2 different shRARRES2. One-way ANOVA. Treatment of chemerin-silenced 786-O cells with 50 nM recombinant chemerin protein rescued the lipid deposition defect. Two-tailed student's t-test. (FIG. 3B) Oil-Red-O staining of lipid droplets of tumors harvested from mice implanted with 786-O cells infected with lentivirus encoding either sgCONT or sgRARRES2 (4×). (FIG. 3C) Heatmap of RNA-sequencing for 786-O cells infected with lentivirus encoding either shGFP control (shGFP 1-3) or shRARRES2 (shRARRES2 1-8). (FIG. 3D) Gene set enrichment analysis of RNA-seq demonstrated downregulation of lipid metabolism pathways, including lysophospholipid pathway, steroid hormone biosynthesis pathway and ether lipid metabolism pathway. (FIG. 3E) Expression of CPT1A, ACAD9, DBI, ACOT7, FABP7, RORC, APOE, SMPD3, and PLIN4, as measured by qRT-PCR, in the 786-O cells infected with lentivirus targeting shGFP or 2 different shRARRES2. One-way ANOVA. (FIG. 3F) Fatty acid species that were significantly reduced in metabolite profiling of 786-O cells infected with lentivirus encoding shGFP or 2 different shRARRES2. Two-tailed student's t-test. (FIG. 3G) Etomoxir treatment (50 μM and 100 μM) rescues tumorigenesis in chemerin-knockdown 786-O cells, measured by EdU flow cytometry. Two-tailed student's t-test. Error bars represent SEM of three independent experiments and three technical replica per experiment. *p<0.05; **p<0.01; ***p<0.001; ****p<0.0001.

FIGS. 4A-4K show that chemerin-dependent lipid homeostasis pathways affects ferroptosis. (FIG. 4A) Heatmap from untargeted lipidomics of 786-O infected with lentivirus targeting shGFP control, shRARRES2-1, and shRARRES2-2. Blue, low expression; red, high expression. (FIG. 4B) Volcano plot showing the differentially expressed lipid metabolites in shGFP in comparison to shRARRES2. Blue and red circles represent lipid species that were significantly downregulated and upregulated, respectively, in RARRES2-targeted cells. (FIG. 4C) Proportion of oxidized or breakdown versus intact lipid levels in shRARRES2 cells compared to shGFP. (FIG. 4D) Fold change of acylcarnitine species in RARRES2-targeted 786-O cells versus shGFP. (FIG. 4E) Metabolic Pathway Analysis using KEGG human metabolome database that shows the significantly upregulated metabolic pathways according to untargeted lipidomics and metabolomics data after chemerin knockdown. (FIG. 4F) Heatmap from untargeted lipidomics of 786-O infected with lentivirus targeting shGFP (n=4) or shRARRES2 (including shRARRES2-1 and shRARRES2-2, n=8) showing lower glycerophospholipid species in chemerin deficient cells. Blue, low expression; red, high expression. PA, phosphatidic acid; PC, phosphatidylcholine; PG, phosphatidylglycerol. (FIG. 4G) Heatmap from untargeted lipidomics showing higher oxidized glycerophospholipid species in chemerin deficient cells. Blue, low expression; red, high expression. OxPC, oxidized phosphatidylcholine; OxLPC, oxidized lysophosphatidylcholine; OxPE, oxidized phosphatidylethanolamine; OxTG, oxidized triacylglycerols. (FIG. 4H) Heatmap from untargeted lipidomics showing higher lysophosphatidylcholine, the breakdown product of phosphatidylcholine, in chemerin deficient cells. Blue, low expression; red, high expression. LPC, lysophosphatidylcholine; Cer, ceramides. (FIG. 4I) Saturation index of different lipid species after chemerin knockdown. Stacked bar chart shows different saturation abundances of significant lipid classes in chemerin knockdown cells compared to controls. DB, double bond. (FIG. 4J) Lipid reactive oxygen species (ROS) measurement by BODIPY 581/591 C-11 in 786-O infected with lentivirus encoding either shGFP or shRARRES2. One-way ANOVA. (FIG. 4K) 786-O cells infected with lentivirus encoding either shGFP or shRARRES2, were treated with 2 different ferroptosis inhibitors (1 μM Ferrostatin-1 or 0.5 μM Liproxstatin-1). Two-tailed student's t-test. Error bars represent SEM of three independent experiments and three technical replica per experiment. *p<0.05; **p<0.01; ***p<0.001; ****p<0.0001.

FIGS. 5A-5E show genetic deletion of chemerin in ccRCC cells affects mitochondrial OXPHOS. (FIG. 5A) Metabolic profiling of CoQ (CoQ6, CoQ7, CoQ8, and CoQ10) and farnesyl-diphosphate from mass spectrometry analysis of 786-O cells infected with lentivirus targeting shGFP control or shRARRES2. Four technical replicates were performed. (FIG. 5B) Steady-state levels of mitochondrial individual OXPHOS complexes were evaluated by BN-PAGE analysis of whole cell extracts prepared in the presence of 1% lauryl maltoside (LM) and probed with antibodies against complex I subunit NDUFA9, complex II subunit SDHA, complex III subunit CORE2, complex IV subunit COX1 and complex V subunit ATP5. (FIG. 5C) Signals from (FIG. 5B) were quantified and normalized by VDAC using the histogram function of Adobe Photoshop on digitalized images. Error bars represent the mean±SD of three independent experiments with two technical replicates. Unpaired T-test with Welch's correction. (FIG. 5D) NADH substrate-driven cellular respiratory rates expressed as percentile of shGFP control endogenous 02 consumption. Error bars represent the mean±SD of three independent experiments with two technical replicates. Unpaired t-test with Welch's correction. (FIG. 5E) ATP measurement following chemerin knockdown in 786-O cells. One-way ANOVA. Error bars represent SEM of three independent experiments and three technical replica per experiment. *p<0.05; **p<0.01; ***p<0.001; ****p<0.0001; ns, not significant.

FIGS. 6A-6J show that chemerin expression is VHL dependent and regulates HIF expression. (FIG. 6A) scRNA-seq data from human normal transplant kidney, normal kidney tissue adjacent to tumor, ccRCC specimens with wild-type VHL and ccRCC specimens with VHL mutation, presented as UMAP plots. (FIG. 6B) Immunoblots of lysates from 786-O transfected with VHL plasmids. (FIG. 6C) Relative mRNA expression of chemerin in 786-O and 760-P cells infected with lentivirus encoding shGFP or shHIF2α. Two-tailed student's t-test. (FIG. 6D) Relative mRNA expression of chemerin in 786-O and 769-P cells transfected with control siRISC or siKLF6. Two-tailed student's t-test. (FIG. 6E) Expression of HIF1α and HIF2α after chemerin knockdown in 786-O cells, measured by qRT-PCR. One-way ANOVA. (FIG. 6F) Immunoblots of lysates from 786-O and 769-P for HIF2α after infection with lentivirus targeting shGFP or 2 different shRARRES2. (FIG. 6G) Gene set enrichment analysis of RNA-sequencing of 786-O infected with lentivirus encoding either shGFP or shRARRES2, demonstrating downregulation of hypoxia associated pathways. Blue, low expression; red, high expression. (FIG. 6H) Relative mRNA expression of HIF target genes, VEGF, LOX and IGFBP3 after chemerin knockdown in 786-O cells, measured by qRT-PCR. One-way ANOVA. (FIG. 6I) Relative mRNA expression of HIF1α and HIF2α in 786-O after incubation with 500 μM linoleic acid or 500 μM palmitic acid for 24 hours, measured by qRT-PCR. One-way ANOVA. (FIG. 6J) Proposed mechanism of chemerin regulation of ccRCC tumorigenesis. HIF2α, hypoxia inducible factor 2 alpha; MUFA, monounsaturated fatty acid; PUFA, polyunsaturated fatty acid; ROS, reactive oxygen species; SFA, saturated fatty acid; TCA cycle, tricarboxylic acid cycle. Error bars represent SEM of three independent experiments and three technical replica per experiment. *p<0.05; **p<0.01; ***p<0.001; ****p<0.0001.

FIGS. 7A-7G show that monoclonal antibody (mAb) targeting chemerin reduces ccRCC tumor burden. (FIG. 7A) Cell viability assay of 786-O and HK-2 cells treated with RARRES2 mAb (upper panels) and IgG mAb control (lower panels). Two-way repeated measures ANOVA was used for statistical analysis with Tukey correction. (FIG. 7B) ORO staining quantification in 786-O cells treated with either IgG mAb or RARRES2 mAb. Two tailed student's t-test. (FIG. 7C) Relative lipid ROS measured using BODIPY 581/591 C11 assay in 786-O cells treated with either IgG mAb or RARRES2 mAb. Two tailed student's t-test. (FIG. 7D) Representative bioluminescence imaging of before (Day 0) and Day 28 post-treatment in mice receiving 20 mg/kg of either mAb (n=6 each arm), after 786-O was implanted orthotopically under the left kidney capsule of nude mice. (FIG. 7E) Quantification of bioluminescence imaging of before (day 0) and day 52 post-treatment in mice receiving 20 mg/kg of either mAb (n=6 each arm), after 786-O cells were implanted orthotopically under the left kidney capsule of nude mice. One-tailed student's t-test. (FIG. 7F) Tumor weight measurement (n=6 each group) at the end of the assay. Student's t-test. (FIG. 7G) Representative kidney tumor from (FIG. 7E). Error bars represent SEM of three independent experiments and three technical replica per experiment. *p<0.05; **p<0.01; ***p<0.001; ****p<0.0001.

FIG. 8 shows that cells are classified into different cell clusters and display distinctive gene signatures, Related to FIG. 1. Heatmap displays the differentially expressed genes in each cluster of cell populations identified from single-cell RNA-sequencing. The differentially expressed genes were utilized to identify cell populations (Table 1). Purple, low expression; yellow, high expression.

FIGS. 9A-9E show that ccRCC tumor cluster shows specific tumor gene signatures, as compared to other cell clusters, Related to FIG. 1. (FIG. 9A) Uniform manifold approximation and projection (UMAP) includes all cells from normal transplant kidney, ccRCC tumors, normal adjacent kidney tissues and wild-type VHL ccRCC tumors. Tumors cluster annotations according to gene markers from scRNA-seq in FIG. 8. (FIG. 9B) The same UMAP as in (FIG. 9A) with selected cell types annotated; the cell types were determined according to the expression level of the representative gene markers indicated in FIG. 8. (FIG. 9C) The cluster annotations according to the tissue types. (FIG. 9D) Gene set enrichment analysis verifies the upregulation of hypoxia, glycolysis, myc-targets and mTORC1 signaling related genes in ccRCC tumor clusters as compared to epithelium. Purple, low expression; red, high expression. (FIG. 9E) Gene set enrichment analysis shows no significant correlation of hypoxia and NFKB related genes between two subclusters of ccRCC tumor cells. Purple, low expression; red, high expression.

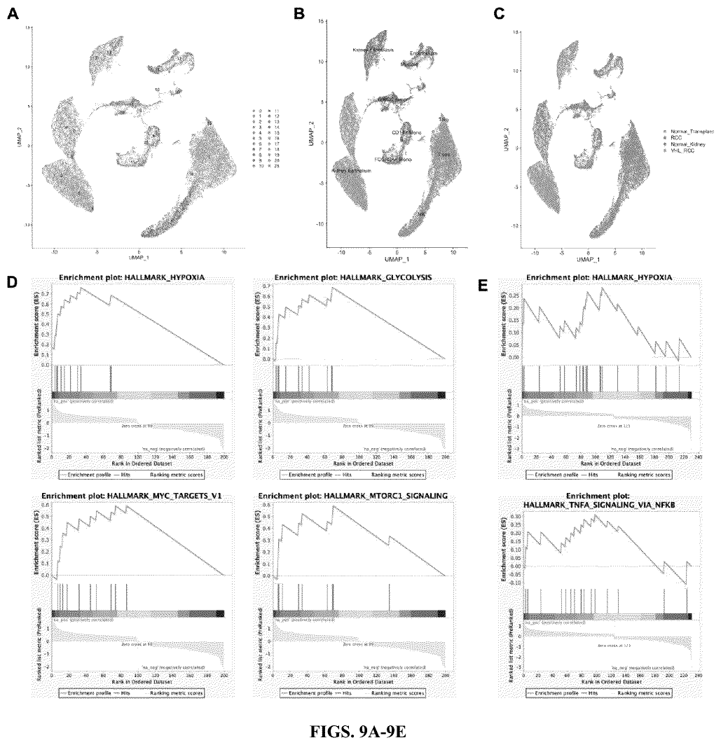

FIGS. 10A-10H show that chemerin protein is detected in ccRCC tissue; and chemerin expression is not correlated with survival of patients with papillary renal cell carcinoma and chromophobe renal cell carcinoma, Related to FIG. 1. (FIG. 10A) Kaplan-Meier curve showing no correlation of higher mRNA expression of chemerin with survival of papillary renal cell carcinoma patients in the TCGA KIRP dataset using upper and lower third cohorts. Log rank analysis. (FIG. 10B) Kaplan-Meier curve showing no correlation of higher mRNA expression of chemerin with survival of chromophobe renal cell carcinoma patients in the TCGA KICH dataset using upper and lower third cohorts. Log rank analysis. (FIG. 10C) ROC curve of plasma chemerin. Area under the curve is 0.99 with sensitivity of 98.31% and specificity of 95.83% at the cut-off value of 121.6 ng/mL. (FIG. 10D) Relative expression of chemerin in normal kidney tissue adjacent to ccRCC tumor, ccRCC tumor tissue, fat tissue adjacent to the ccRCC tumor tissue, and fat tissue distant to the ccRCC tumor tissue. Two-tailed student's t-test. (FIG. 10E) Immunohistochemistry staining (IHC) of chemerin protein expression in a tumor microarray containing 30 ccRCC tissues with matched cancer adjacent kidney tissue. Each column represents different pairs of tumor (T) and normal (N) samples. Normalized intensity of staining was quantified on the right. (FIG. 10F) Representative 10× pictures of ccRCC and normal adjacent kidney tissues in IHC sections in (FIG. 10E). (FIG. 10G) Representative Oil-Red-O pictures of ccRCC with low and high expression of chemerin (10×). (FIG. 10H) Plasma chemerin concentration in normal healthy individuals (n=24), patients with ccRCC tumor (n=59) and patients with papillary RCC (non-ccRCC) (n=26). Mann-Whitney U-test. Error bars represent SD. *p<0.05; **p<0.01; ***p<0.001; ****p<0.0001.

FIGS. 11A-11J show that chemerin is functionally important for ccRCC growth, Related to FIG. 2. (FIG. 11A) Relative mRNA expression of chemerin in 786-O following knockdown with 3 independent, non-overlapping shRNAs targeting RARRES2 (shRARRES2-1, shRARRES2-2, shRARRES2-3) or control shGFP. One-way ANOVA. (FIG. 11B) Relative mRNA expression of chemerin in 786-O following knockdown with 3 independent, non-overlapping shRNAs targeting RARRES2 (shRARRES2-1, shRARRES2-2, shRARRES2-3) or control shGFP at Day 0, Day 7 and Day 14 post-infection. One-way ANOVA. (FIG. 11C) Relative chemerin protein concentration in media of 786-O and 769-O following knockdown with 3 independent, non-overlapping shRNAs targeting RARRES2 (shRARRES2-1, shRARRES2-2, shRARRES2-3) or control shGFP 72 hours post-infection. One-way ANOVA. Error bars represent SD. (FIG. 11D) Immunoblot of different ccRCC cell lines (A-498, RCC4, 786-O, 769-P, UOK101 and HK-2) demonstrating relative chemerin protein expression. (FIG. 11E) Relative mRNA expression of chemerin in HK-2 following knockdown with 3 independent, non-overlapping shRNAs targeting RARRES2 (shRARRES2-1, shRARRES2-2, shRARRES2-3) or control shGFP. One-way ANOVA. (FIG. 11F) Cell proliferation assay from HK-2 cells infected with shGFP control or 3 different shRNAs encoding lentivirus targeting chemerin (shRARRES2-1, shRARRES2-2, shRARRES2-3). Two-way repeated measures ANOVA with Geisser-Greenhouse correction. (FIG. 11G) Colony forming assay from 786-O cells infected with shGFP control or 3 different shRNAs encoding lentivirus targeting chemerin (shRARRES2-1, shRARRES2-2, shRARRES2-3), measured by number of colonies formed from initial 100 cells after 2 weeks. Two-way repeated measures ANOVA with Geisser-Greenhouse correction. (FIG. 11H) Subcutaneous tumor volume measurement in nude mice implanted with 769-P cells which were infected with lentivirus encoding either shGFP (n=8) or 2 different shRARRES2 (n=8 in each arm). Two-way measures ANOVA with Dunnett's multiple comparison test correction. (FIG. 11I) Immunoblot of subcutaneous tumors derived from sgCONT and sgRARRES2 from FIG. 2G demonstrating complete knockout of chemerin protein. (FIG. 11J) Ki67 IHC of subcutaneous tumors derived from sgCONT and sgRARRES2 from FIG. 2G demonstrating reduced tumor cell proliferation in vivo (10×). Representative images were quantified and shown on the right. Error bars represent SEM of three independent experiments and three technical replica per experiment. *p<0.05, **p<0.01, ***p<0.001, ****p<0.0001.

FIGS. 12A-12D show lipid metabolism pathways alteration in chemerin-deficient ccRCC, Related to FIG. 3. (FIG. 12A) Volcano plot showing commonly differentially expressed genes in between 786-O cells infected with lentivirus encoding either shGFP control (n=3) or combined shRARRES2 clones (n=8). (FIG. 12B) Heatmaps showing the downregulated genes in chemerin-targeted versus control 786-O cells in different lipid metabolism pathways. Purple, low expression; red, high expression. (FIG. 12C) Representative EdU flow cytometry plots for FIG. 3G. (FIG. 12D) 50 μM Etomoxir treatment rescues proliferation defect in chemerin-knockdown 786-O cells, measured by cell proliferation assay. Two-way repeated measures ANOVA with Geisser-Greenhouse correction. Error bars represent SD. *p<0.05, **p<0.01, ***p<0.001, ****p<0.0001.

FIGS. 13A-13G show untargeted lipidomics profiling in 786-O with chemerin inhibition, Related to FIG. 4. (FIG. 13A) Overview of lipid profiles between two independent clones of shRARRSE2 obtained through untargeted lipidomic analysis as annotated by the LipidMatch (guided in-source fragment annotation) algorithm. (FIG. 13B) Clustering between control and two independent clones of shRARRSE2 obtained through untargeted lipidomic analysis as annotated by the LipidMatch (Based on MS/MS based confirmation) algorithm. (FIG. 13C) Percentage of each lipid species identified in mass spectrometry analysis. (FIG. 13D) The lipid species identified in intact and oxidized/breakdown lipid categories. (FIG. 13E) Structure of phosphatidylcholine (PC) and lysophosphatidylcholine (LPC). (FIG. 13F) Heatmap from untargeted lipidomics showing lower sphingomyelin species and increased breakdown product sphingosine in chemerin deficient cells. Blue, low expression; red, high expression. DG, diacylglcerols; SM, sphingomyelin; So, sphingosine. (FIG. 13G) Heatmap from untargeted lipidomics showing reduction in glycerolipid monogalactosyldiacylglycerol (MGDG) in chemerin deficient cells. Blue, low expression; red, high expression. MG, monoglyceride; MGDG, monogalactosyldiacylglycerol.

FIGS. 14A-14C show that chemerin inhibition reduces mitochondrial OXPHOS complex IV but do not cause overall structural change to mitochondria, Related to FIG. 5. (FIG. 14A) Steady-state levels of mitochondrial individual OXPHOS complex III subunit CORE2 and complex IV subunit COX1 in 786-O cells infected with lentivirus targeting shGFP or 2 different shRARRES2. This was evaluated by BN-PAGE analysis of whole cell extracts prepared in the presence of 1% lauryl maltoside (LM) and probed with antibodies. Four technical replicates were performed. (FIG. 14B) Seahorse Cell Mito Stress Test in 786-O following knockdown with 2 independent, non-overlapping shRNAs targeting RARRES2 (shRARRES2-1, shRARRES2-2) or control shGFP. The oxygen consumption rate (OCR) in chemerin-silenced cell is lower than in the control cell. (FIG. 14C) Confocal imaging of mitochondrial structure stained with Mitotrex and Tom20 in 786-O cells infected with lentivirus targeting shGFP or 2 different shRARRES2 (10×). Error bars represent SD. *p<0.05, **p<0.01, ***p<0.001, ****p<0.0001.

FIGS. 15A-15I show chemerin regulation by KLF6, Related to FIG. 6. (FIG. 15A) Tumor clusters of human normal kidney, ccRCC specimens with wild-type VHL and ccRCC specimens with VHL mutation from scRNA-seq data, presented as UMAP plots. (FIG. 15B) Relative mRNA expression of RARRES2 in 786-O and 786-O VHL cells, measured by qRT-PCR. Two-tailed student's t-test. (FIG. 15C) Relative mRNA expression of HIF2α in 786-O and 769-P cells infected with shGFP control or shRNAs encoding lentivirus targeting HIF2α, confirming knockdown of HIF2α in FIG. 6C. Two-tailed student's t-test. (FIG. 15D) Fold change of RARRES2 mRNA expression in 786-O with KLF6 knockdown as compared to control, extracted from RNA-seq data (28). (FIG. 15E) Relative mRNA expression of KLF6 in 786-O and 769-P cells transfected with control siRISC or siRNA targeting KLF6 (siKLF6), confirming knockdown of KLF6 in FIG. 6D. Two-tailed student's t-test. (FIG. 15F) Immunoblot of lysate confirming knockdown of KLF6 in 786-O and 769-P cells transfected with control siRISC or siKLF6 in FIG. 6D. (FIG. 15G) Correlation of chemerin and KLF6 expression in multiple ccRCC cell lines interrogated from the Cancer Dependency Map database. The R2 value of the linear correlation is 0.83 with p-value of 0.0302. (FIG. 15H) Heatmap of downregulated hypoxia pathway genes in 786-O infected with lentivirus encoding either shGFP or shRARRES2. Purple, low expression; red, high expression. (FIG. 15I) Immunoblots of lysates of 786-O and 769-P cells treated with 500 μM linoleic acid (a polyunsaturated fatty acid) or 500 μM palmitate (a saturated fatty acid), showing decreased in HIF2α and chemerin protein after incubation with 500 μM linoleic acid but increased after incubation with 500 μM palmitate. Error bars represent SEM. *p<0.05, **p<0.01, ***p<0.001, ****p<0.0001.

FIG. 16 shows that monoclonal antibody targeting chemerin shows no toxicity in mice, Related to FIG. 7. The weight of mice receiving 20 mg/kg of either mAb (n=6 each arm), after 786-O was implanted orthotopically under the left kidney capsule of nude mice. Weight is recorded weekly starting from the day of tumor implantation.

FIGS. 17A-17E show that CMKLR1 regulates cell growth and lipid metabolism in ccRCC cells. (FIG. 17A) Relative mRNA expression of CMKLR1 in 769-P cell infected with lentivirus encoding either control sgCONT or sgCMKLR1 (sgCMKLR1-1 and sgCMKLR1-2). Two-way repeated measures ANOVA with Geisser-Greenhouse correction. (FIG. 17B) Immunoblot of lysate confirming knockdown of CMKLR1 protein from 769-P cells infected with sgCONT or sgCMKLR1 (sgCMKLR1-1 and sgCMKLR1-2). (FIG. 17C) Cell proliferation assay of 769-P cells infected with lentivirus encoding either sgCONT or sgCMKLR1 (sgCMKLR1-1 and sgCMKLR1-2). Two-way repeated measures ANOVA with Geisser-Greenhouse correction. (FIG. 17D) BODIPY staining of 769-P cells infected with either sgCONT or sgCMKLR1 (sgCMKLR1-1 and sgCMKLR1-2) (upper panel). DAPI staining of nucleus in lower panel (4×). Quantification of BODIPY staining, normalized to DAPI staining in 769-P cells infected with lentivirus encoding either sgCONT or sgCMKLR1 (sgCMKLR1-1 and sgCMKLR1-2). One-way ANOVA. (FIG. 17E) Expression of CPT1A and PLIN4, as measured by qRT-PCR, in the 769-P cells infected with either sgCONT or sgCMKLR1 (sgCMKLR1-1 and sgCMKLR1-2). One-way ANOVA. Error bars represent SD. *p<0.05, **p<0.01, ***p<0.001, ****p<0.0001.

FIG. 18 shows the antibody sequences of RARRES21H1. The sequences in the drawing include SEQ ID NO: 1, SEQ ID NO: 2, SEQ ID NO: 3, and SEQ ID NO: 4.

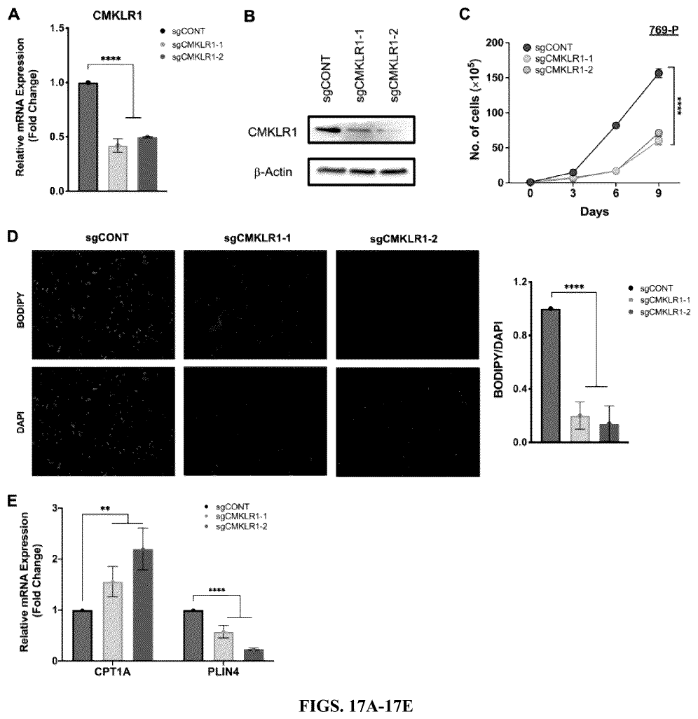

FIG. 19 shows the antibody information. The sequences in FIG. 19 include SEQ ID NO: 81 and SEQ ID NO: 82.

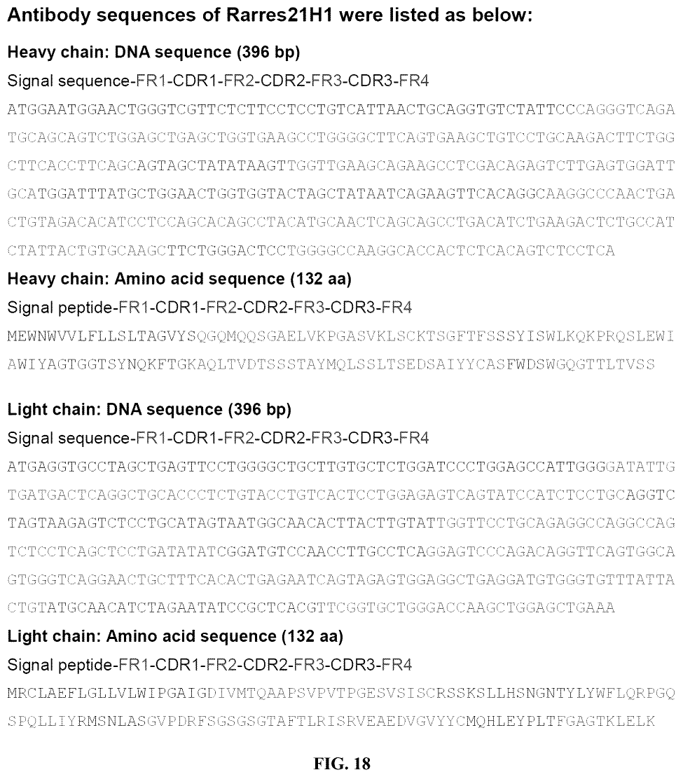

FIG. 20 shows comparison between the antibody and a standard of care drug cabozantinib. It shows that the antibody treatment (20 mg/kg twice per week) of a patient derived xenograft of kidney cancer is as effective as cabo (10 mg/kg) at 5 times per week.

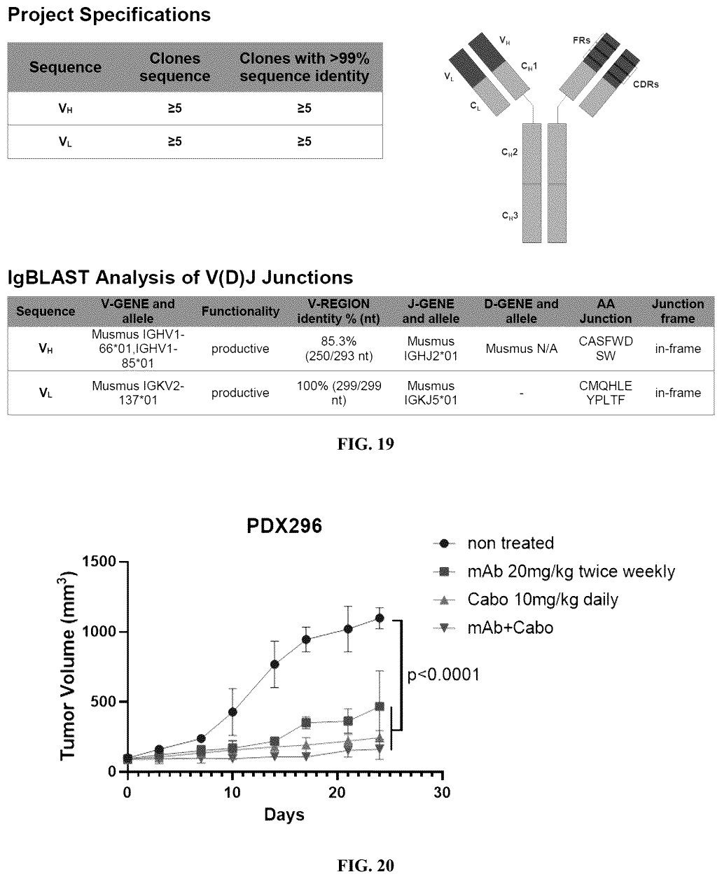

### DETAILED DESCRIPTION

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. As used in this disclosure and in the appended claims, the singular forms “a”, “an”, “the”, include plural referents unless the context clearly dictates otherwise.

The following definitions are provided for the full understanding of terms used in this specification.

### Terminology

The term “about” as used herein when referring to a measurable value such as an amount, a percentage, and the like, is meant to encompass variations of ±20%, ±10%, ±5%, or ±1% from the measurable value.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

As used herein, the term “antigen” refers to a molecule that is capable of binding to an antibody. In some embodiments, the antigen stimulates an immune response such as by production of antibodies specific for the antigen.

As used herein, “specific for” and “specificity” means a condition where one of the molecules is involved in selective binding. Accordingly, an antibody that is specific for one antigen selectively binds that antigen and not other antigens.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof. The antibodies can be tested for their desired activity using the in vitro assays described herein, or by analogous methods, after which their in vivo therapeutic and/or prophylactic activities are tested according to known clinical testing methods. Native antibodies are usually heterotetrameric glycoproteins of about 150,000 daltons, composed of two identical light (L) chains and two identical heavy (H) chains. Each heavy chain has at one end a variable domain (VH) followed by a number of constant domains. Each light chain has a variable domain at one end (VL) and a constant domain at its other end. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

Each antibody molecule is made up of the protein products of two genes: heavy-chain gene and light-chain gene. The heavy-chain gene is constructed through somatic recombination of V, D, and J gene segments. In human, there are 51 VH, 27 DH, 6 JH, 9 CH gene segments on human chromosome 14. The light-chain gene is constructed through somatic recombination of V and J gene segments. There are 40 Vκ, 31 Vλ, 5 Jκ, 4 Jλ gene segments on human chromosome 14 (80 VJ). The heavy-chain constant domains that correspond to the different classes of immunoglobulins are called α, δ, ε, γ, and μ, respectively. The “light chains” of antibodies from any vertebrate species can be assigned to one of two clearly distinct types, called kappa (κ) and lambda (λ), based on the amino acid sequences of their constant domains.

As used herein, the term “antibody or antigen binding fragment thereof” or “antibody or fragments thereof” encompasses chimeric antibodies and hybrid antibodies, with dual or multiple antigen or epitope specificities, and fragments, such as F(ab′)2, Fab′, Fab, Fv, sFv, scFv, nanoantibody and the like, including hybrid fragments. Thus, fragments of the antibodies that retain the ability to bind their specific antigens are provided. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

The terms “antigen binding site”, “binding site” and “binding domain” refer to the specific elements, parts or amino acid residues of a polypeptide, such as an antibody, that bind the antigenic determinant or epitope.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations, κ and λ light chains refer to the two major antibody light chain isotypes.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

The term “biological sample” as used herein means a sample of biological tissue or fluid. Such samples include, but are not limited to, tissue isolated from animals. Biological samples can also include sections of tissues such as biopsy and autopsy samples, frozen sections taken for histologic purposes, blood, plasma, serum, sputum, stool, tears, mucus, hair, and skin. Biological samples also include explants and primary and/or transformed cell cultures derived from patient tissues. A biological sample can be provided by removing a sample of cells from an animal, but can also be accomplished by using previously isolated cells (e.g., isolated by another person, at another time, and/or for another purpose), or by performing the methods as disclosed herein in vivo. Archival tissues, such as those having treatment or outcome history can also be used.

The term “tissue” refers to a group or layer of similarly specialized cells which together perform certain special functions. The term “tissue” is intended to include, blood, blood preparations such as plasma and serum, bones, joints, muscles, smooth muscles, lung tissues, and organs.

The term “biomarker” refers to a biological marker characterizing a phenotype. A biomarker typically includes a gene or a gene product. Depending on the gene, “detecting a biomarker” may include detecting altered gene expression, epigenetic modifications, germ-line or somatic mutations, etc. In case of a gene product, “detecting a biomarker” may mean detecting the presence, quantity or change in quantity of a cell surface marker or a soluble compound, etc. Detecting a biomarker (e.g., detecting chemerin) may also include detecting gene expression (mRNA or protein) or a metabolite reflective of a gene's expression or activity.

The term “cancer” as used herein is defined as disease characterized by the rapid and uncontrolled growth of aberrant cells. Cancer cells can spread locally or through the bloodstream and lymphatic system to other parts of the body, Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

The term “cancer cells” and “tumor cells” are used interchangeably to refer to cells derived from a cancer or a tumor, or from a tumor cell line or a tumor cell culture. The term “primary tumor” refers to a tumor growing at the site of the cancer origin. The term “metastatic tumor” refers to a secondary tumor growing at the site different from the site of the cancer origin.

The term “primary tumor” refers to a tumor growing at the site of the cancer origin.

The term “metastatic tumor” refers to a secondary tumor growing at the site different from the site of the cancer origin.

As used herein, the term “metastasis” is meant to refer to the process in which cancer cells originating in one organ or part of the body, with or without transit by a body fluid, and relocate to another part of the body and continue to replicate. Metastasized cells can subsequently form tumors which may further metastasize. Metastasis thus refers to the spread of cancer, from the part of the body where it originally occurred, to other parts of the body.

The term “CDR” as used herein refers to the “complementarity determining regions” of the antibody which consist of the antigen binding loops. (Kabat E. A. et al., (1991) Sequences of proteins of immunological interest. NIH Publication 91-3242). Each of the two variable domains of an antibody Fv fragment contain, for example, three CDRs.

A “control” is an alternative subject or sample used in an experiment for comparison purposes. A control can be “positive” or “negative.”

The term “hypervariable region” or “HVR”, as used herein, refers to each of the regions of an antibody variable domain which are hypervariable in sequence and/or form structurally defined loops (“hypervariable loops”). HVRs generally comprise amino acid residues from the hypervariable loops and/or from the complementarity determining regions (CDRs), the latter being of highest sequence variability and/or involved in antigen recognition. CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen-binding regions. The amino acid sequence boundaries of a CDR can be determined by one of skill in the art using any of a number of known numbering schemes, including those described by Kabat et al., supra (“Kabat” numbering scheme): Al-Lazikani et al., 1997. J. Mol. Biol., 273:927-948 (“Chothia” numbering scheme); MacCallum et al., 1996, J. Mol. Biol, 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme); each of which is incorporated by reference in its entirety.

“Effective amount” encompasses, without limitation, an amount that can ameliorate, reverse, mitigate, prevent, or diagnose a symptom or sign of a medical condition or disorder. Unless dictated otherwise, explicitly or by context, an “effective amount” is not limited to a minimal amount sufficient to ameliorate a condition. The severity of a disease or disorder, as well as the ability of a treatment to prevent, treat, or mitigate, the disease or disorder can be measured, without implying any limitation, by a biomarker or by a clinical parameter. In some embodiments, the term “effective amount of a recombinant antibody” refers to an amount of a recombinant antibody sufficient to prevent, treat, or mitigate a cancer.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA.

The term as used herein “engineered” and other grammatical forms thereof may refer to one or more changes of nucleic acids, such as nucleic acids within the genome of an organism. The term “engineered” may refer to a change, addition and/or deletion of a gene. Engineered cells can also refer to cells that contain added, deleted, and/or changed genes.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.)

The “fragments” or “functional fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the functional fragment must possess a bioactive property, such as binding to tumor antigen (e.g., chemerin or a fragment thereof), and/or suppressing tumor growth.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The term “increased” or “increase” as used herein generally means an increase by a statically significant amount; for example, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

“Inhibit”, “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inhibitors” of expression or of activity are used to refer to inhibitory molecules, respectively, identified using in vitro and in vivo assays for expression or activity of a described target protein, e.g., ligands, antagonists, and their homologs and mimetics. Inhibitors are agents that, e.g., inhibit expression or bind to, partially or totally block stimulation or protease activity, decrease, prevent, delay activation, inactivate, desensitize, or down regulate the activity of the described target protein, e.g., antagonists. A control sample (untreated with inhibitors) are assigned a relative activity value of 100%. Inhibition of a described target protein is achieved when the activity value relative to the control is about 80%, optionally 50% or 25, 10%, 5% or 1%.

As used herein, the terms “nanobody”, “V_{H}H”, “V_{H}H antibody fragment” and “single domain antibody” are used indifferently and designate a variable domain of a single heavy chain of an antibody of the type found in Camelidae, which are without any light chains, such as those derived from Camelids as described in PCT Publication No. WO 94/04678, which is incorporated by reference in its entirety.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

“Recombinant” used in reference to a gene refers herein to a sequence of nucleic acids that are not naturally occurring in the genome of the bacterium. The non-naturally occurring sequence may include a recombination, substitution, deletion, or addition of one or more bases with respect to the nucleic acid sequence originally present in the natural genome of the bacterium.

“Nucleotide,” “nucleoside,” “nucleotide residue,” and “nucleoside residue,” as used herein, can mean a deoxyribonucleotide, ribonucleotide residue, or another similar nucleoside analogue. A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

The method and the system disclosed here including the use of primers, which are capable of interacting with the disclosed nucleic acids, such as the antigen barcode as disclosed herein. In certain embodiments the primers are used to support DNA amplification reactions. Typically, the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically, the disclosed primers hybridize with the disclosed nucleic acids or region of the nucleic acids or they hybridize with the complement of the nucleic acids or complement of a region of the nucleic acids.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “polypeptide” refers to a compound made up of a single chain of D- or L-amino acids or a mixture of D- and L-amino acids joined by peptide bonds.

The term “host cell” as used herein shall refer to primary subject cells trans-formed to produce a particular recombinant protein, such as an antibody as described herein, and any progeny thereof. It should be understood that not all progeny are exactly identical to the parental cell (due to deliberate or inadvertent mutations or differences in environment), however, such altered progeny are included in these terms, so long as the progeny retain the same functionality as that of the originally transformed cell. The term “host cell line” refers to a cell line of host cells as used for expressing a recombinant gene to produce recombinant polypeptides such as recombinant antibodies. The term “cell line” as used herein refers to an established clone of a particular cell type that has acquired the ability to proliferate over a prolonged period of time. Such host cell or host cell line may be maintained in cell culture and/or cultivated to produce a recombinant polypeptide.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

As used herein, “operatively linked” can indicate that the regulatory sequences useful for expression of the coding sequences of a nucleic acid are placed in the nucleic acid molecule in the appropriate positions relative to the coding sequence so as to effect expression of the coding sequence. This same definition is sometimes applied to the arrangement of coding sequences and/or transcription control elements (e.g. promoters, enhancers, and termination elements), and/or selectable markers in an expression vector. The term “operatively linked” can also refer to the arrangement of polypeptide segments within a single polypeptide chain, where the individual polypeptide segments can be, without limitation, a protein, fragments thereof, linking peptides, and/or signal peptides. The term operatively linked can refer to direct fusion of different individual polypeptides within the single polypeptides or fragments thereof where there are no intervening amino acids between the different segments as well as when the individual polypeptides are connected to one another via one or more intervening amino acids.

“Operatively linked”, as used herein, means at least two chemical structures joined together in such a way as to remain linked through the various manipulations described herein. Typically the functional moiety and the encoding oligonucleotide are linked covalently via an appropriate linking group. The linking group is at least a bivalent moiety with a site of attachment for the oligonucleotide and a site of attachment for the functional moiety. For example, when the functional moiety is a polyamide compound, the polyamide compound can be attached to the linking group at its N-terminus, its C-terminus or via a functional group on one of the side chains. The linking group is sufficient to separate the polyamide compound and the oligonucleotide by at least one atom and in some embodiments by more than one atom. In some embodiments, the linking group is sufficiently flexible to allow the polyamide compound to bind target molecules in a manner which is independent of the oligonucleotide.

The term “specificity” refers to the number of different types of antigens or antigenic determinants to which a particular antigen-binding molecule (such as the recombinant antibody of the invention) can bind. As used herein, the term “specifically binds,” as used herein with respect to a recombinant antibody refers to the recombinant antibody's preferential binding to one or more epitopes as compared with other epitopes. Specific binding can depend upon binding affinity and the stringency of the conditions under which the binding is conducted. In one example, an antibody specifically binds an epitope when there is high affinity binding under stringent conditions.

It should be understood that the specificity of an antigen-binding molecule (e.g., recombinant antibodies) can be determined based on affinity and/or avidity. The affinity, represented by the equilibrium constant for the dissociation of an antigen with an antigen-binding molecule (K_{D}), is a measure for the binding strength between an antigenic determinant and an antigen-binding site on the antigen-binding molecule: the lesser the value of the K_{D}, the stronger the binding strength between an antigenic determinant and the antigen-binding molecule (alternatively, the affinity can also be expressed as the affinity constant (K_{A}), which is 1/K_{D}). As will be clear to the skilled person (for example on the basis of the further disclosure herein), affinity can be determined in a manner known per se, depending on the specific antigen of interest. Avidity is the measure of the strength of binding between an antigen-binding molecule (such as recombinant antibodies) and the pertinent antigen. Avidity is related to both the affinity between an antigenic determinant and its antigen binding site on the antigen-binding molecule and the number of pertinent binding sites present on the antigen-binding molecule. Typically, antigen-binding proteins (such as the recombinant antibodies of the invention) will bind to their antigen with a dissociation constant (K_{D}) of 10⁻⁵ to 10⁻¹² moles/liter or less, and preferably 10⁻⁷ to 10⁻¹² moles/liter or less, and more preferably 10⁻⁸ to 10⁻¹² moles/liter.

“Therapeutically effective amount” refers to the amount of a composition such as recombinant antibody that will elicit the biological or medical response of a tissue, system, animal, or human that is being sought by the researcher, veterinarian, medical doctor or other clinician over a generalized period of time. In some embodiments, a desired response is reduction of coronaviral titers in a subject. In some embodiments, the desired response is mitigation of coronavirus infection and/or related symptoms. In some instances, a desired biological or medical response is achieved following administration of multiple dosages of the composition to the subject over a period of days, weeks, or years. The therapeutically effective amount will vary depending on the composition, the disorder or conditions and its severity, the route of administration, time of administration, rate of excretion, drug combination, judgment of the treating physician, dosage form, and the age, weight, general health, sex and/or diet of the subject to be treated. The therapeutically effective amount of recombinant antibodies as described herein can be determined by one of ordinary skill in the art.

A therapeutically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. Measured or measurable parameters include clinically detectable markers of disease, for example, elevated or depressed levels of a biological marker, such as decreased size of tumor, and/or prolonged survival of a subject. It will be understood that the total daily usage of the compositions and formulations as disclosed herein will be decided by the attending physician within the scope of sound medical judgment. The exact amount required will vary depending on factors such as the type of disease being treated.

The terms “treat,” “treating,” “treatment,” and grammatical variations thereof as used herein, include partially or completely delaying, alleviating, mitigating or reducing the intensity of one or more attendant symptoms of a cancer or condition and/or alleviating, mitigating or impeding one or more causes of a cancer. Treatments according to the invention may be applied preventively, prophylactically, palliatively or remedially. Prophylactic treatments are administered to a subject prior to onset (e.g., before obvious signs of cancer), during early onset (e.g., upon initial signs and symptoms of cancer), after an established development of cancer. Prophylactic administration can occur for several minutes to months prior to the manifestation of cancer.

As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

### Chemerin Inhibitors and Methods of Treatment

In some aspects, disclosed herein is a method of treating/preventing/mitigating kidney cancer, treating/preventing/mitigating kidney cancer metastasis, and/or treating/preventing/mitigating kidney cancer relapse in a subject, comprising administering to the subject a therapeutically effective amount of an inhibitor of chemerin. In some embodiments, the kidney cancer is clear cell renal cell carcinoma.

“Chemerin” refers herein to a polypeptide that, in humans, is encoded by the RARRES2 gene. In some embodiments, the chemerin polypeptide is that identified in one or more publicly available databases as follows: HGNC: 9868; NCBI Entrez Gene: 5919; Ensembl: ENSG00000106538; OMIM®: 601973; UniProtKB/Swiss-Prot: Q99969. In some embodiments, the chemerin polypeptide comprises the sequence of SEQ ID NO: 45, or a polypeptide sequence having at or greater than about 80%, about 85%, about 90%, about 95%, or about 98% homology with SEQ ID NO: 45, or a polypeptide comprising a portion of SEQ ID NO: 45. The chemerin polypeptide of SEQ ID NO: 45 may represent an immature or “pre-processed form of mature chemerin, and accordingly, included herein are mature or processed portions of the chemerin polypeptide in SEQ ID NO: 80.

In some embodiments, the inhibitor of chemerin is a polypeptide, a polynucleotide, a small molecule, or a gene editing tool.

In some embodiments, the polypeptide is a recombinant antibody. In some embodiments, the recombinant antibody is a humanized antibody.

In some embodiments, the recombinant antibody comprises a heavy chain variable domain (VH) comprising a heavy chain complementarity-determining region (CDRH)1, a CDRH2, and/or a CDRH3, wherein
the CDRH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 8 or a fragment thereof,
the CDRH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 9 or a fragment thereof, and
the CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 10 or a fragment thereof.

In some embodiments, the recombinant antibody comprises a heavy chain variable domain (VH) comprising a heavy chain complementarity-determining region (CDRH)1, a CDRH2, and/or a CDRH3, wherein
the CDRH1 comprises the sequence of SEQ ID NO: 8 or a fragment thereof,
the CDRH2 comprises the sequence of SEQ ID NO: 9 or a fragment thereof, and
the CDRH3 comprises the sequence of SEQ ID NO: 10 or a fragment thereof.

In some embodiments, the VH comprises a sequence at least 60% (for example, at least 60%, at least 65%, at least 70%, at least 75%, at least 80%, at least 85%, at least 90%, at least 95%, at least 96%, at least 97%, at least 98%, at least 99%) SEQ ID NO: 3 or a fragment thereof. In some embodiments, the VH comprises the sequence of SEQ ID NO: 3 or a fragment thereof.

In some embodiments, the recombinant antibody disclosed herein comprises a light chain variable domain (VL) comprising a light chain complementarity-determining region (CDRL)1, a CDRL2, and/or a CDRL3, wherein
the CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 14 or a fragment thereof,
the CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 15 or a fragment thereof, and
the CDRL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 16 or a fragment thereof.

In some embodiments, the recombinant antibody of any preceding aspect comprises a light chain variable domain (VL) comprising a light chain complementarity-determining region (CDRL)1, a CDRL2, and/or a CDRL3, wherein
the CDRL1 comprises the sequence of SEQ ID NO: 14 or a fragment thereof,
the CDRL2 comprises the sequence of SEQ ID NO: 15 or a fragment thereof, and
the CDRL3 comprises the sequence of SEQ ID NO: 16 or a fragment thereof.

In some embodiments, the VL comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 4 or a fragment thereof. In some embodiments, the VL comprises the sequence of SEQ ID NO: 4 or a fragment thereof.

In some embodiments, the chemerin inhibitor disclosed herein is a polynucleotide. In some embodiments, the polynucleotide is an siRNA or an shRNA. In some embodiments, the shRNA comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 77, 78, or 79, or a fragment there of.

In some embodiments, the chemerin inhibitor disclosed herein is a gene editing tool. In some embodiments, the gene editing tool is a CRISPR/Cas endonuclease (Cas)9 system.

“CRISPR system” herein refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. CRISPR systems are known in the art. See, e.g., U.S. Pat. No. 8,697,359, incorporated by reference herein in its entirety.

A gRNA is a component of the CRISPR/Cas system. A “gRNA” (guide ribonucleic acid) herein refers to a fusion of a CRISPR-targeting RNA (crRNA) and a trans-activation crRNA (tracrRNA), providing both targeting specificity and scaffolding/binding ability for Cas9 nuclease. A “crRNA” is a bacterial RNA that confers target specificity and requires tracrRNA to bind to Cas9. A “tracrRNA” is a bacterial RNA that links the crRNA to the Cas9 nuclease and typically can bind any crRNA. The sequence specificity of a Cas DNA-binding protein is determined by gRNAs, which have nucleotide base-pairing complementarity to target DNA sequences. The native gRNA comprises a Specificity Determining Sequence (SDS), which specifies the DNA sequence to be targeted. At least a portion of the target DNA sequence is complementary to the SDS of the gRNA. For Cas9 to successfully bind to the DNA target sequence, a region of the target sequence is complementary to the SDS of the gRNA sequence and is immediately followed by the correct protospacer adjacent motif (PAM) sequence. In some embodiments, an SDS is 100% complementary to its target sequence. In some embodiments, the SDS sequence is less than 100% complementary to its target sequence and is, thus, considered to be partially complementary to its target sequence. For example, a targeting sequence may be 99%, 98%, 97%, 96%, 95%, 94%, 93%, 92%, 91%, or 90% complementary to its target sequence.

In some embodiments, the CRISPR/Cas9 system used herein comprises a guide RNA comprising a sequence at least 60% (for example, at least 60%, at least 65%, at least 70%, at least 75%, at least 80%, at least 85%, at least 90%, at least 95%, at least 96%, at least 97%, at least 98%, at least 99%) identical to any one of SEQ ID NOs: 17-40 or a fragment thereof. In some embodiments, the CRISPR/Cas9 system comprises a guide RNA comprising a sequence selected from the group consisting of SEQ ID NOs: 17-40 or a fragment thereof.

Also disclosed herein is a method of treating/preventing/mitigating kidney cancer relapse, treating/preventing/mitigating kidney cancer metastasis, and/or treating/preventing/mitigating kidney cancer in a subject, wherein said method comprises:
obtaining a biological sample from the subject;
determining if the biological sample has an increased level of chemerin relative to a reference control; and
initiating a cancer therapy to treat the subject if the biological sample has an increased level of chemerin.

Also disclosed herein is a method of diagnosing clear cell renal cell carcinoma in a subject, wherein said method comprises:
obtaining a biological sample from the subject;
measuring the level of chemerin in the biological sample; and
determining that the subject has clear cell renal cell carcinoma if the biological sample has an increased level of chemerin relative to a reference control.

In some embodiments, the kidney cancer is clear cell renal cell carcinoma.

In some embodiments, the method further comprises initiating a cancer therapy to treat the subject diagnosed as having kidney cancer (e.g., clear cell renal cell carcinoma).

In some embodiments, the biological sample has an increased level of a chemerin polypeptide. In some embodiments, the biological sample has an increased level of a chemerin polynucleotide. The term “reference control” refers to a level in detected in a subject in general or a study population (e.g., healthy control).

In some embodiments, the cancer therapy is administration of an anti-cancer agent. In some embodiments, the cancer therapy is radiation therapy, thermal ablation, or cryosurgery.

In some embodiments, the anti-cancer agent is cabozantinib, axitinib, sunitinib, sorafenib, or pazopanib. In some embodiments, the anti-cancer agent is an immune checkpoint inhibitor (e.g., an anti-PD-1 inhibitor, anti-PD-L1 inhibitor, anti-PD-L2 inhibitor, or ant-CTLA4 inhibitor). In some embodiments, the immune checkpoint inhibitor is selected from the group consisting of Ipilimumab, Pembrolizumab, Nivolumab, Atezolizumab, Avelumab, and Durvalumab. In some embodiments, the anti-cancer agent is an mTOR inhibitor (e.g., everolimus or temsirolimus). In some embodiments, the anti-cancer agent is a HIF2a inhibitor, such as MK-6482. In some embodiments, the anti-cancer agent comprises an inhibitor of chemerin disclosed herein.

In some embodiments, the inhibitor of chemerin is a polypeptide, a polynucleotide, a small molecule, or a gene editing tool.

In some embodiments, the polypeptide is a recombinant antibody. In some embodiments, the recombinant antibody is a humanized antibody. In some embodiments, the recombinant antibody is a single domain antibody.

In some embodiments, the recombinant antibody comprises a heavy chain variable domain (VH) comprising a heavy chain complementarity-determining region (CDRH)1, a CDRH2, and/or a CDRH3, wherein
the CDRH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 8 or a fragment thereof,
the CDRH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 9 or a fragment thereof, and
the CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 10 or a fragment thereof.

In some embodiments, the recombinant antibody comprises a heavy chain variable domain (VH) comprising a heavy chain complementarity-determining region (CDRH)1, a CDRH2, and/or a CDRH3, wherein
the CDRH1 comprises the sequence of SEQ ID NO: 8 or a fragment thereof,
the CDRH2 comprises the sequence of SEQ ID NO: 9 or a fragment thereof, and
the CDRH3 comprises the sequence of SEQ ID NO: 10 or a fragment thereof.

In some embodiments, the VH comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 3 or a fragment thereof. In some embodiments, the VH comprises the sequence of SEQ ID NO: 3 or a fragment thereof.

In some embodiments, the recombinant antibody of any preceding aspect comprises a light chain variable domain (VL) comprising a light chain complementarity-determining region (CDRL)1, a CDRL2, and/or a CDRL3, wherein
the CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 14 or a fragment thereof,
the CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 15 or a fragment thereof, and
the CDRL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 16 or a fragment thereof.

In some embodiments, the recombinant antibody of any preceding aspect comprises a light chain variable domain (VL) comprising a light chain complementarity-determining region (CDRL)1, a CDRL2, and/or a CDRL3, wherein
the CDRL1 comprises the sequence of SEQ ID NO: 14 or a fragment thereof,
the CDRL2 comprises the sequence of SEQ ID NO: 15 or a fragment thereof, and
the CDRL3 comprises the sequence of SEQ ID NO: 16 or a fragment thereof.

In some embodiments, the VL comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 4 or a fragment thereof. In some embodiments, the VL comprises the sequence of SEQ ID NO: 4 or a fragment thereof.

In some embodiments, the polynucleotide is an siRNA or an shRNA. In some embodiments, the gene editing tool is a CRISPR/Cas endonuclease (Cas)9 system. In some embodiments, the CRISPR/Cas9 system comprises a guide RNA comprising a sequence selected from the group consisting of SEQ ID NOs: 17-40 or a fragment thereof.

Also disclosed herein is a recombinant antibody comprising a heavy chain variable domain (VH) comprising a heavy chain complementarity-determining region (CDRH)1, a CDRH2, and/or a CDRH3, wherein
the CDRH1 comprises a sequence at least 80% identical to SEQ ID NO: 8 or a fragment thereof,
the CDRH2 comprises a sequence at least 80% identical to SEQ ID NO: 9 or a fragment thereof, and
the CDRH3 comprises a sequence at least 80% identical to SEQ ID NO: 10 or a fragment thereof.

In some embodiments, the recombinant antibody comprises a heavy chain variable domain (VH) comprising a heavy chain complementarity-determining region (CDRH)1, a CDRH2, and/or a CDRH3, wherein
the CDRH1 comprises the sequence of SEQ ID NO: 8 or a fragment thereof,
the CDRH2 comprises the sequence of SEQ ID NO: 9 or a fragment thereof, and
the CDRH3 comprises the sequence of SEQ ID NO: 10 or a fragment thereof.

In some embodiments, the recombinant antibody further comprises a light chain variable domain (VL) comprising a light chain complementarity-determining region (CDRL)1, a CDRL2, and/or a CDRL3, wherein
the CDRL1 comprises a sequence at least 80% identical to SEQ ID NO: 14 or a fragment thereof,
the CDRL2 comprises a sequence at least 80% identical to SEQ ID NO: 15 or a fragment thereof, and
the CDRL3 comprises a sequence at least 80% identical to SEQ ID NO: 16 or a fragment thereof.

In some embodiments, the recombinant antibody of any preceding aspect comprises a light chain variable domain (VL) comprising a light chain complementarity-determining region (CDRL)1, a CDRL2, and/or a CDRL3, wherein
the CDRL1 comprises the sequence of SEQ ID NO: 14 or a fragment thereof,
the CDRL2 comprises the sequence of SEQ ID NO: 15 or a fragment thereof, and
the CDRL3 comprises the sequence of SEQ ID NO: 16 or a fragment thereof.

Methods for determining antibody sequences and antigen-antibody specificities are known in the art. See, e.g., International Publication Number: WO 2020/033164, incorporated by reference.

Also disclosed herein is a method of treating kidney cancer (e.g., clear cell renal cell carcinoma) in a subject comprising administering to the subject a therapeutically effective amount of a recombinant polynucleotide comprising a nucleic acid sequence encoding the recombinant antibody disclosed herein.

### Antibody Compositions

Also disclosed herein is a recombinant antibody comprising a heavy chain variable domain (VH) comprising a heavy chain complementarity-determining region (CDRH)1, a CDRH2, and/or a CDRH3, wherein
the CDRH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 8 or a fragment thereof,
the CDRH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 9 or a fragment thereof, and
the CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 10 or a fragment thereof.

In some embodiments, the recombinant antibody comprises a heavy chain variable domain (VH) comprising a heavy chain complementarity-determining region (CDRH)1, a CDRH2, and/or a CDRH3, wherein
the CDRH1 comprises the sequence of SEQ ID NO: 8 or a fragment thereof,
the CDRH2 comprises the sequence of SEQ ID NO: 9 or a fragment thereof, and
the CDRH3 comprises the sequence of SEQ ID NO: 10 or a fragment thereof.

In some embodiments, the VH comprises a sequence at least 60% (for example, at least 60%, at least 65%, at least 70%, at least 75%, at least 80%, at least 85%, at least 90%, at least 95%, at least 96%, at least 97%, at least 98%, at least 99%) SEQ ID NO: 3 or a fragment thereof. In some embodiments, the VH comprises the sequence of SEQ ID NO: 3 or a fragment thereof.

In some embodiments, the recombinant antibody further comprises a light chain variable domain (VL) comprising a light chain complementarity-determining region (CDRL)1, a CDRL2, and/or a CDRL3, wherein
the CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 14 or a fragment thereof,
the CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 15 or a fragment thereof, and
the CDRL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 16 or a fragment thereof.

In some embodiments, the recombinant antibody of any preceding aspect comprises a light chain variable domain (VL) comprising a light chain complementarity-determining region (CDRL)1, a CDRL2, and/or a CDRL3, wherein
the CDRL1 comprises the sequence of SEQ ID NO: 14 or a fragment thereof,
the CDRL2 comprises the sequence of SEQ ID NO: 15 or a fragment thereof, and
the CDRL3 comprises the sequence of SEQ ID NO: 16 or a fragment thereof.

In some embodiments, the VL comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 4 or a fragment thereof. In some embodiments, the VL comprises the sequence of SEQ ID NO: 4 or a fragment thereof.

In some embodiments, the recombinant antibody disclosed herein is a humanized antibody. In some embodiments, the recombinant antibody is a single domain antibody.

Also disclosed herein is a recombinant polynucleotide comprising a nucleic acid sequence encoding the recombinant antibody of any preceding aspect.

Also disclosed herein is an expression vector comprising the recombinant polynucleotide of any preceding aspect.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day. In some embodiments, the active agent is administered to the subject at a dosage ranging from 0.1 μg/kg body weight to 100 g/kg body weight. In some embodiments, the active agent is administered to the subject at a dosage of from 1 μg/kg to 10 g/kg, from 10 μg/kg to 1 g/kg, from 10 μg/kg to 500 mg/kg, from 10 μg/kg to 100 mg/kg, from 10 μg/kg to 10 mg/kg, from 10 μg/kg to 1 mg/kg, from 10 μg/kg to 500 μg/kg, or from 10 μg/kg to 100 μg/kg body weight. The dosage of administration for the active agent disclosed herein can be from about 0.01 mg/kg body weight to about 100 mg/kg body weight. In some examples, the dosage is about 0.01 mg/kg body weight, about 0.05 mg/kg body weight, about 0.1 mg/kg body weight, about 0.5 mg/kg body weight, about 1 mg/kg body weight, about 1.5 mg/kg body weight, about 2 mg/kg body weight, about 2.5 mg/kg body weight, about 3 mg/kg body weight, about 3.5 mg/kg body weight, about 4 mg/kg body weight, about 4.5 mg/kg body weight, about 5 mg/kg body weight, about 5.5 mg/kg body weight, about 6 mg/kg body weight, about 6.5 mg/kg body weight, about 7 mg/kg body weight, about 7.5 mg/kg body weight, about 8 mg/kg body weight, about 8.5 mg/kg body weight, about 9 mg/kg body weight, about 9.5 mg/kg body weight, about 10 mg/kg body weight, about 11 mg/kg body weight, about 12 mg/kg body weight, about 13 mg/kg body weight, about 14 mg/kg body weight, about 15 mg/kg body weight, about 20 mg/kg body weight, about 25 mg/kg body weight, about 30 mg/kg body weight, about 35 mg/kg body weight, about 40 mg/kg body weight, about 45 mg/kg body weight, about 50 mg/kg body weight, about 55 mg/kg body weight, about 60 mg/kg body weight, about 65 mg/kg body weight, about 70 mg/kg body weight, about 75 mg/kg body weight, about 80 mg/kg body weight, about 85 mg/kg body weight, about 90 mg/kg body weight, about 95 mg/kg body weight, or about 100 mg/kg body weight. Dosages above or below the range cited above may be administered to the individual patient if desired.

### Antibodies

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with a chemerin polypeptide. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, scFv, and the like, including hybrid fragments. Thus, fragments of the antibodies that retain the ability to bind their specific antigens are provided. For example, fragments of antibodies which maintain chemerin binding activity are included within the meaning of the term “antibody or fragment thereof” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

### Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### Administration of Antibodies

Administration of the antibodies can be done as disclosed herein. Nucleic acid approaches for antibody delivery also exist. The broadly neutralizing anti-chemerin antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

### EXAMPLES

The following examples are set forth below to illustrate the antibodies, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Background

ccRCC tumors almost uniformly have alterations in the von Hippel-Lindau (VHL) gene, including mutations, deletions or hypermethylation, rendering the VHL protein (pVHL) functionally inactive. In normal oxygen conditions (normoxia), the hypoxia-inducible factors (HIFs) are hydroxylated by one of three PHD enzymes, leading to recognition and binding to pVHL. pVHL, which functions as an E3 ubiquitin ligase, subsequently targets HIFs for proteasomal degradation. In contrast, during hypoxia, or in ccRCC with a dysfunctional pVHL, HIF1α and HIF2α are not degraded and accumulate in cells. The HIFα subunits (predominantly HIF2α in ccRCC) then translocate into nucleus, bind to hypoxia responsive elements (HRE) and drive transcription of a host of hypoxia-responsive adaptation genes. Some of the HIF targets include vascular endothelial growth factor (VEGF), glycolysis genes, and CCND1, which contribute to the angiogenesis, Warburg metabolism, and hyperproliferative phenotypes of ccRCC.

The “clear cell” type of RCC is characterized by high lipid and glycogen content. Lipid homeostasis in cells is a tightly regulated process to maintain normal cellular functions, ranging from lipid synthesis, uptake, and subcellular distribution or compartmentalization. Of all, the storage of fatty acid (FA) in lipid droplets, which is the histologically identifiable phenotype of ccRCC, is an important equilibrating mechanism to prevent lipotoxicity. FAs can be used as building blocks for complex lipids, and can also be used as substrates for beta-oxidation (β-oxidation) in mitochondria to yield energy. Dysregulated metabolism is a well-established hallmark of cancer and an enticing target for cancer management. In ccRCC, reports have shown that lipid metabolism contributes to metabolic reprogramming and unbridled cell growth. Yet the exact mechanism and the significance of the lipid-storage phenotype remain poorly understood. The potential to exploit a metabolic vulnerability as a therapeutic strategy in lipid-associated cancers also remains largely unexplored.

In the past few decades, the cases of obesity and obesity-related disorders have been increasing steadily in both the developed and developing countries, leading to the declaration by the World Health Organization (WHO) of a global obesity epidemic in 1999. Evidence has also established that tumorigenesis is escalated by adiposity, showing a direct link between metabolism and cancer. In fact, obesity (BMI at least 30 kg/m²) is a known risk factor for ccRCC, and perinephric fat invasion has been associated with poor disease outcome in ccRCC. Hypertrophic obesity (increase in adipocyte size), hyperplastic obesity (increase in adipocyte number) as well as increased intracellular lipids can contribute to an increase in circulating pro-inflammatory adipokines that act on peripheral tissues and stimulate key cellular biological processes, recently found to include tumorigenesis. Realizing the intimate interplay between adiposity and cancer, this study tested if adipokines plays detrimental roles in driving tumorigenesis in lipid-laden ccRCC. Notably, the roles and mechanisms of obesity-related factors in increasing cancer risk remain unclear.

By combining single-cell resolution transcriptomic data, metabolomic analyses and multiple ccRCC model systems, this study discovered that the adipokine chemerin, which is encoded by the retinoic acid receptor responder 2 (RARRES2) gene, is overexpressed in ccRCC due to both an autocrine, tumor-cell-dependent mechanism, as well as obesity-dependent paracrine production, and plays important roles in regulating lipid metabolism and tumorigenesis. This study found that chemerin depletion results in dramatic metabolic rewiring in which excessive lipid oxidation leads to mitochondrial dysfunction and ferroptosis. Chemerin controls expression of over 300 genes, most notably HIF1α and HIF2α, through a fatty acid-dependent mechanism. The secretory nature of chemerin makes its elevation amenable to detection in the plasma of ccRCC patients, and further to interruption through the use of a monoclonal antibody. Accordingly, this study finds targeting chemerin to be effective in controlling orthotopic xenograft tumors in vivo. Together, the data provide a biological link between obesity, suppression of fatty acid oxidation, and tumor growth in lipid-laden ccRCC, showing a molecular marker and therapeutic target to improve standard therapeutic approaches.

### Example 2. Results

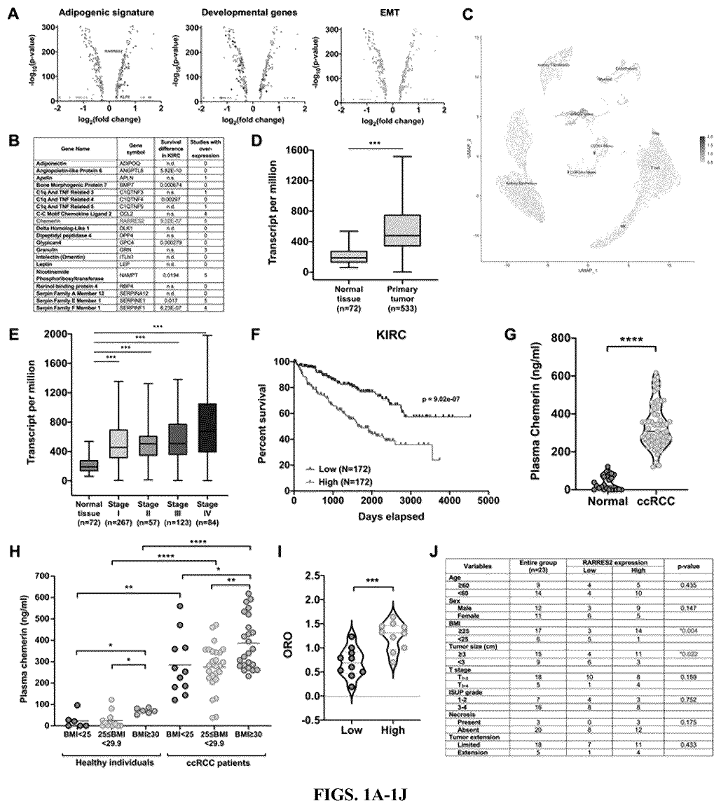

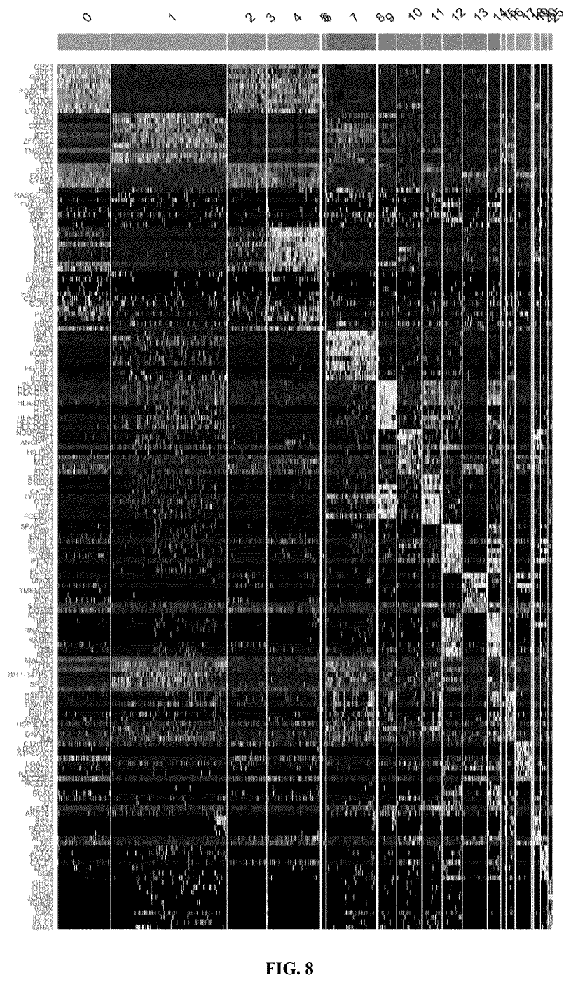

Chemerin is elevated in ccRCC patients through obesity- and tumor-dependent processes. In analysis of bulk tumors, ccRCC cells show evidence of transdifferentiation into adipocytes, which are known to secrete a variety of factors that both maintain their own adipocyte functions, and promote adipocyte differentiation of distant cells. To validate transdifferentiation in ccRCC tumor cells, this study utilized single cell RNA-sequencing (scRNA-seq) of tumors (n=3) and normal kidney tissues (n=5), and by comparing the tumor cell and normal kidney epithelium clusters (FIG. 8, 9A-9E, Table 1), this study verified that ccRCC tumor cells display a reduced renal epithelial gene signature in favor of an adipogenic signature (FIG. 1A). Tumor cells also demonstrated de-differentiation and epithelial-to-mesenchymal transition, in agreement with the literature (FIG. 1A).

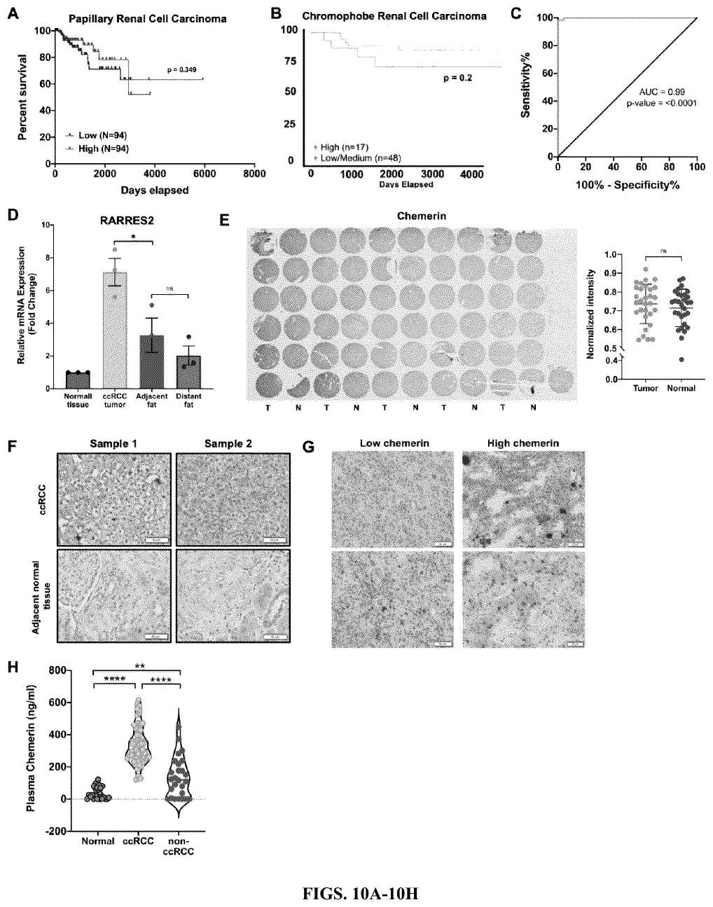

In an effort to identify a potential autocrine adipokine that can be critical for promoting and maintaining ccRCC, in silico data mining was performed using The Cancer Genome Atlas (TCGA) as well as Oncomine databases, and association of 100 known adipokines with ccRCC was examined, some of which are shown in FIG. 1B. The adipokines were ranked according to the number of studies showing an overexpression profile in ccRCC, and the correlation with patient survival; the adipokine chemerin, stood out for further investigation. From scRNA-seq data, it was determined that tumor expression of chemerin arises from ccRCC tumor cells rather than other cells in the tumor microenvironment, agreeing with a possible autocrine pathway (FIG. 1C, Table 2). In TCGA, chemerin mRNA expression is higher in ccRCC clinical samples (n=533) as compared to adjacent normal kidney tissues (n=72) (FIGS. 1D, 1E); and higher chemerin expression portends a negative prognosis in ccRCC patients (upper third versus lower third) (FIG. 1F). In contrast, chemerin expression is either not associated with outcome in papillary renal cell carcinoma patients (pRCC) (FIG. 10A) or chromophobe renal cell carcinoma (FIG. 10B) when considering upper and lower thirds, or can even define a better outcome when considering upper and lower fifths.

Since chemerin is notably overexpressed in ccRCC tumors, the next experiment measured plasma chemerin levels and investigated the possibility of using chemerin as a biomarker for ccRCC patients. This study found that chemerin protein levels are significantly elevated in patient plasma relative to healthy individuals (FIG. 1G). The area under the curve (AUC) was 0.99 at the cut-off value of 121.6 ng/mL, demonstrating a sensitivity of 98.31% and specificity of 95.83% (FIG. 10C). Because previous studies have indicated that chemerin is positively correlated with adiposity, patient BMI among the patient samples was considered and it was found that obese patients (BMI≥30) had significantly higher circulating chemerin levels than overweight patients (25≤BMI<30) or low BMI patients (BMI<25) (FIG. 1H). This confirmed the notion that the paracrine chemerin production in ccRCC is obesity-dependent. Importantly, plasma chemerin remains elevated when comparing low BMI ccRCC patients to low BMI healthy individuals, and high BMI patients to high BMI controls (FIG. 1H). This study then assessed chemerin production from tumors, normal adjacent kidney tissue, tumor-adjacent fat, and fat distant to the tumor by performing quantitative reverse transcription-polymerase chain reaction (qRT-PCR) to determine the greatest contributor of chemerin. Significantly elevated RARRES2 mRNA was found in tumor samples versus normal tissue; and that adjacent and distant fat produced equally higher levels than normal kidney, but not as much as the tumor itself (FIG. 10D). The expression changes were validated with immunohistochemistry staining of chemerin protein using a tumor microarray (TMA) with 30 ccRCC samples and associated adjacent normal kidney sections (FIGS. 10E-10F). While chemerin was clearly higher in tumors, higher than expected signals in the “normal” regions was observed, possibly due to possible diffusion of the soluble protein from nearby tumor. Fresh frozen tumor samples were then stained with the lipophilic dye Oil-Red-O (ORO), and ORO uptake was compared with plasma chemerin and a significant positive correlation was found, indicating an association between plasma chemerin, regardless of source, and lipid levels in ccRCC tumors (FIG. 1I, FIG. 10G). Furthermore, the plasma chemerin levels are also correlated with tumor size independently of BMI (FIG. 1J). Again, this study assessed chemerin in a non-clear cell subtype (pRCC), and while circulating levels were elevated over controls, they remained significantly lower than ccRCC (FIG. 10H). Together, the data validate that chemerin is a potentially relevant adipokine in ccRCC.

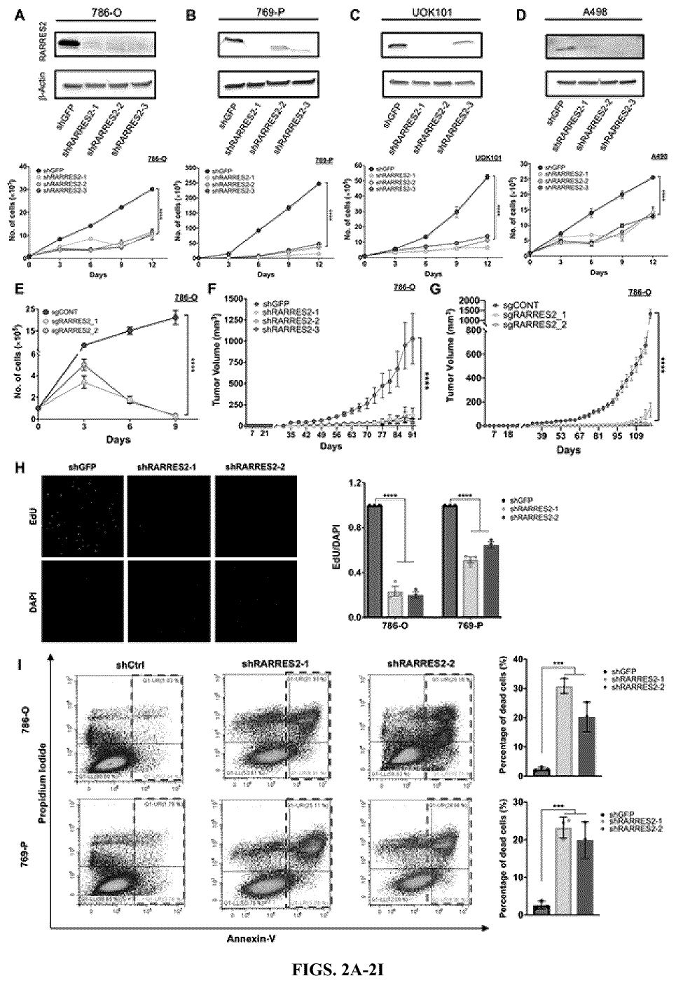

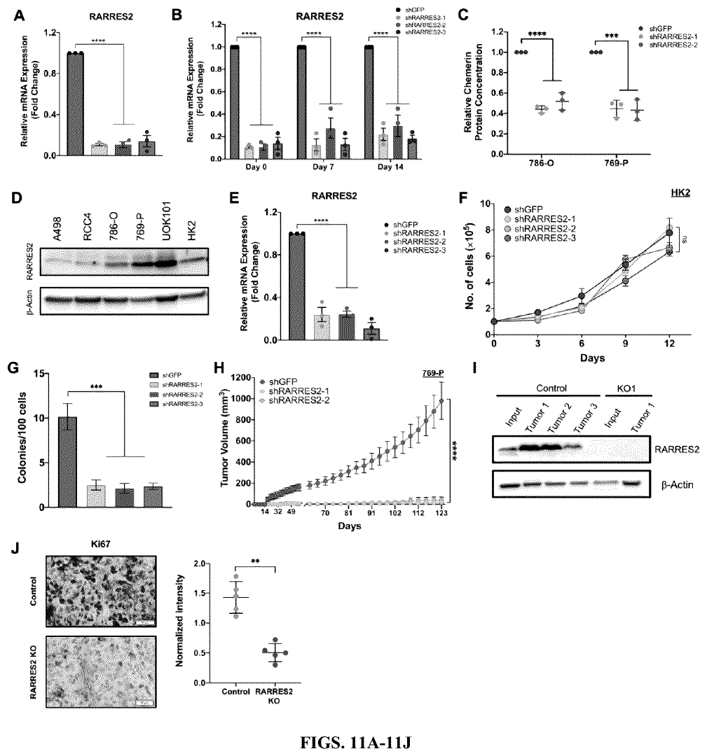

Chemerin sustains ccRCC tumor growth. To functionally dissect a contribution of chemerin in ccRCC, three different shRNAs were used to stably knockdown the chemerin gene RARRES2 in four different ccRCC cell lines: 786-O, 769-P, UOK101 and A-498 (FIGS. 2A-2D, upper panels; FIGS. 11A-11C). All of these ccRCC cell lines contain detectable amounts of chemerin protein (FIG. 11D). All ccRCC isogenic cell lines demonstrated impaired cell proliferation capacity after chemerin knockdown compared to shGFP controls (FIGS. 2A-2D, lower panels); but not HK-2 cells, which are immortalized proximal tubule epithelial cells (FIGS. 11E-11F). The magnitude of inhibition of proliferation appears to correlate with overall chemerin expression levels (i.e., greatest effect in UOK101 and 769-P). This study also observed a reduction in colony forming ability in 786-O cells after chemerin knockdown, another surrogate marker for reduced proliferation ability (FIG. 11G). To eliminate potential off-target effects of shRNAs, CRISPR-Cas9 technology was utilized to knockout chemerin, and the cells showed an even more pronounced impairment of proliferative capacity, where almost all cells died by Day 9 of the assay (FIG. 2E). To investigate whether chemerin is important for ccRCC tumorigenesis in vivo, the 786-O and 769-P cell models infected with either shGFP control or shRARRES2 viruses were transplanted subcutaneously into the flanks of nude mice. Similarly, two unique CRISPR-Cas9 clones of 786-O cells with chemerin knocked-out, and a polyclonal 786-O population with control sgRNA, were also implanted subcutaneously into the flanks of nude mice. Tumors with chemerin inhibition uniformly showed dramatically impaired tumor formation capability in both the knockdown (FIG. 2F, FIG. 11H) and knockout systems (FIG. 2G, FIG. 11I); as well as reduced Ki67 staining in the knockout system (FIG. 11J).

Next, this study interrogated the cellular changes that contribute to the functional phenotypes observed by measuring DNA synthesis as a surrogate for proliferation, and by assessing Annexin V/PI staining for programmed cell death. Knockdown of chemerin in both 786-O and 769-P cells reduced DNA synthesis, derived from reduced EdU incorporation (FIG. 2H); and Annexin-V/PI staining showed increased cell death in the cells after chemerin knockdown (FIG. 2I). Thus, chemerin expression in ccRCC tumor cells is functionally critical for sustaining tumor cell survival.

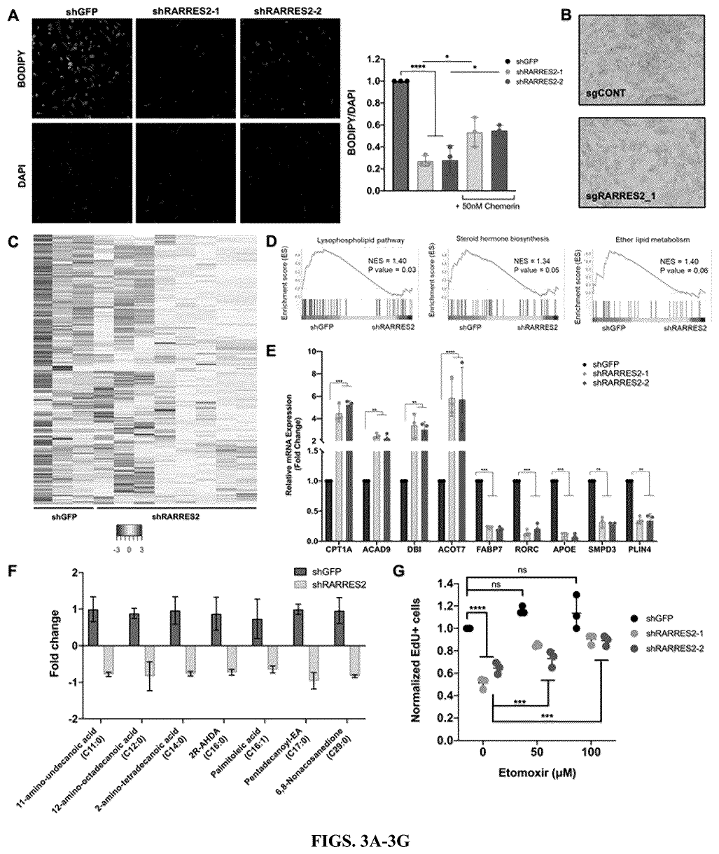

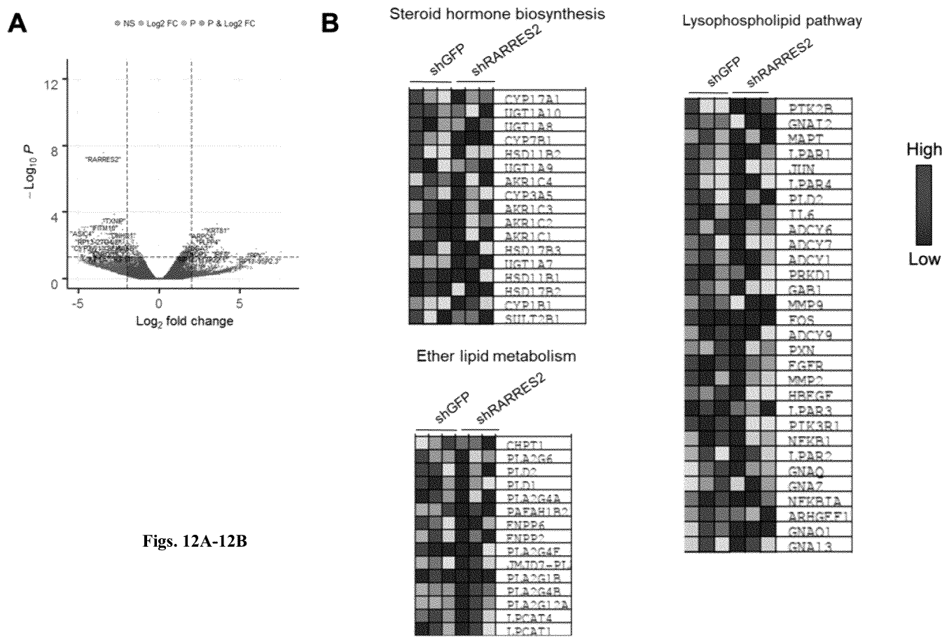

Inhibition of chemerin reprograms lipid metabolism in ccRCC and promotes fatty acid oxidation. To understand whether the chemerin-mediated survival in ccRCC is driven by a change in lipid phenotype, the effect of chemerin on lipid droplets was investigated. To this end, chemerin expression was depleted using shRNAs and the cells were stained with the lipophilic dye BODIPY 493/503 (4,4-Difluoro-1,3,5,7,8-Pentamethyl-4-Bora-3a,4a-Diaza-s-Indacene). Chemerin silencing significantly reduced lipid deposition in the cells; an effect that was partially rescued by incubation of the cells with 50 nM of recombinant chemerin protein for 18 hours (FIG. 3A). The reduction in lipid deposition was also seen in chemerin knockout tumors measured by ORO staining (FIG. 3B). RNA-sequencing of 786-O shGFP control and shRARRES2 cells revealed that chemerin regulates numerous lipid metabolism genes (FIGS. 3C-3E, FIG. 12A). According to genome set enrichment analysis (GSEA) of the transcriptomes, several lipid metabolism pathways were downregulated including the lysophospholipid pathway, steroid hormone biosynthesis and ether lipid metabolism (FIG. 3D, FIG. 12B). Some of the genes were then confirmed by qRT-PCR. The genes tested included the fatty acid oxidation (FAO) enzymes CPT1A, ACAD9, DBI and ACOT7 (FIG. 3E), which were upregulated after chemerin silencing in ccRCC and suggested increased FAO. In contrast, the transcription of the lipid uptake protein FABP7 and the lipid droplet component protein PLIN4 were reduced after chemerin knockdown (FIG. 3E). Consistent with the gene expression changes, there was a reduction in fatty acids measured by mass spectrometry (FIG. 3F), especially the saturated fatty acids (SFA) and monounsaturated fatty acids (MUFA). These results confirm dramatic lipid metabolism alterations after chemerin inhibition, highlighting an important role of chemerin in regulating the ccRCC cellular lipid profile. To determine if decreased tumor cell growth (and tumorigenesis) from chemerin suppression is due to the enhanced mitochondrial β-oxidation of lipids, chemerin-inhibited cells were treated with etomoxir, an inhibitor of the fatty acid mitochondrial transporter carnitine-palmitoyltransferase 1 (CPT1). It was found that etomoxir treatment rescued the reduction in DNA synthesis (FIG. 3G, FIG. 12C) and proliferation defect (FIG. 12D) from chemerin silencing. This observation shows that chemerin-mediated suppression of fatty acid oxidation is necessary to maintain cell proliferation in ccRCC.

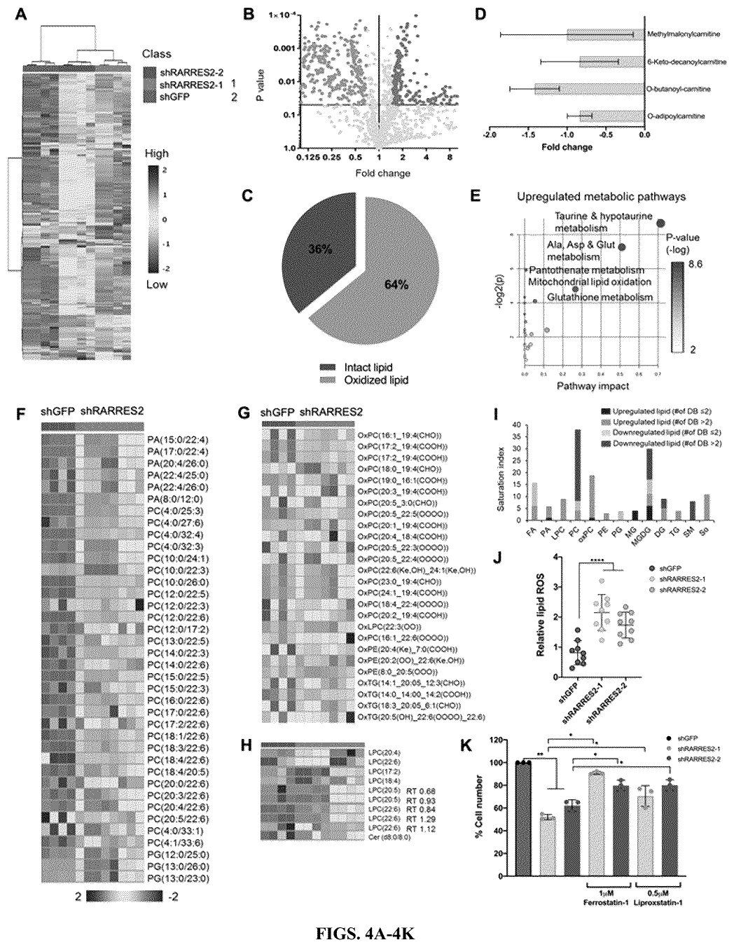

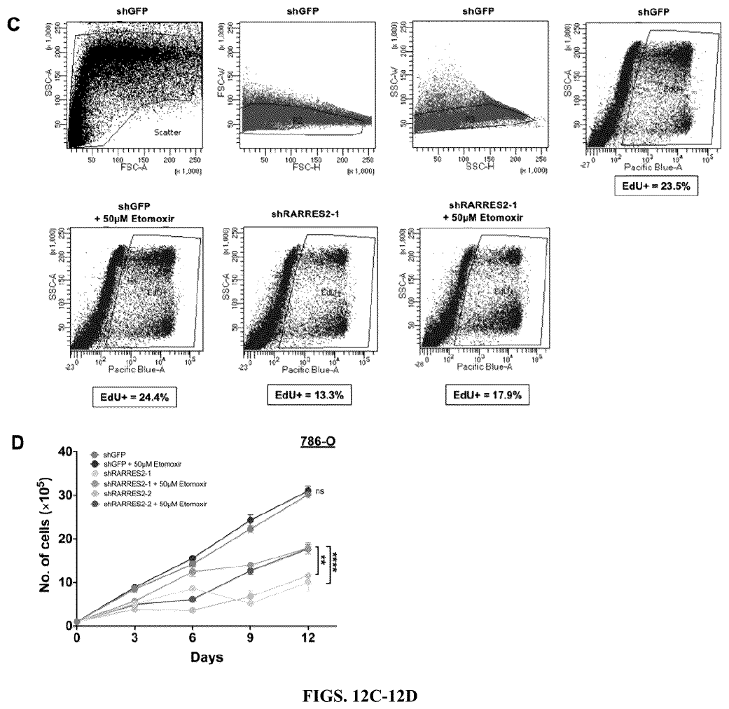

Chemerin inhibition increases oxidized lipid and enhances ferroptosis susceptibility. To further characterize the role of chemerin in lipid metabolism, this study utilized an untargeted lipidomics approach (FIGS. 4A-4B, FIGS. 13A-13B). The lipid species in the analysis included phosphatidylcholine (PC), plasmanyl-PC, phosphatidylethanolamine (PE), phosphatidic acid (PA), ceramides, coenzyme Q (CoQ), acetoacetate (AcAc), phosphatidylglycerol (PG), sphingomyelin, sphingosine, oxidized triacylglycerols, oxidized PE, oxidized PC, monogalactosyldiacylglycerol (MGDG), monoglyceride (MG), lysophosphatidylethanolamine (LPE), lysophosphatidylcholine (LPC), oxidized LPC, and diacylglcerols (DG) (FIGS. 13C-13D). Consistent with transcriptomic findings demonstrating increased FAO, the data show an overall increase in oxidized or breakdown lipids and reduction of intact lipids in 786-O cells transfected with shRNAs targeting chemerin compared to shGFP control (FIG. 4C). For instance, during mitochondrial FAO, carnitine is added to fatty acyl-CoA by CPT1 to form acylcarnitines, which are then transported across mitochondrial inner membrane. Notably, the fatty acyl-carnitine species which feed the downstream FAO cycle were significantly reduced after knockdown of chemerin, validating an increased in oxidation of acylcarnitine species (FIG. 4D). Pathway analysis of the metabolome also revealed upregulated mitochondrial β-oxidation, taurine and hypotaurine metabolism (which promote lipid metabolism) and amino acid pathways that regulate redox balance in the cells after chemerin knockdown (FIG. 4E).

The significant reduction in glycerophospholipid (GPL) species including PA, PC and PG (FIG. 4F) observed in chemerin-deficient cells is accompanied with a complementary increase in corresponding oxidized GPL products (FIG. 4G). Concurrently, the LPC, which are produced after removal of a single acyl group from standard phospholipids with two tails, was increased (FIG. 4H, FIG. 13E). This shows that chemerin-knockdown cells scavenge fatty acid tails from most of the GPL species as substrates for β-oxidation. In addition to oxidation in GPL, other lipid classes were also observed to have undergone oxidation or breakdown. In sphingolipids, intact sphingomyelin was reduced after chemerin knockdown, and the breakdown product sphingosine was significantly increased (FIG. 13F). Another product in sphingolipid metabolism, ceramide, also known to induce cell death, was increased after chemerin knockdown (FIG. 13F). A similar trend was observed in glycerolipids (FIG. 13G).

As accumulation of oxidized lipid products are known to induce ferroptosis in cells, the next experiment sought to investigate if the chemerin knockdown-induced cell death could be explained by ferroptosis. It was found that polyunsaturated fatty acid (PUFA) species, potent ferroptosis inducers, increased after chemerin inhibition (FIG. 4I, orange bars). The increase in PUFA was observed in peroxidized glycerophospholipids, particularly oxidized PC that contain PUFA chains including arachidonic acid (C20:4) and docosahexaenoic acid (C22:6). On the other hand, monounsaturated fatty acid (MUFA) which reduces PUFA incorporation into phospholipid and decreases ferroptosis, was decreased (FIG. 3F). In addition, the increase in LPC is a lipidomic signature of ferroptosis when oxidized PUFA is cleaved from the sn2 position of phospholipid (FIG. 4H, FIG. 13E). PUFAs have been reported to be tumoricidal by enhancing free radical generation. This was also evidenced in cells with chemerin knockdown where an increase in the production of lipid reactive oxygen species (ROS) was observed as measured by BODIPY C-11 581/591 (FIG. 4J). The treatment of chemerin-deficient cells with ferroptosis inhibitors, 1 μM Ferrostatin-1 and 0.5 μM Liproxstatin-1, rescued the proliferation defect (FIG. 4K). Collectively, the results show that ferroptosis is induced in ccRCC after chemerin inhibition by peroxidized lipid species with increased PUFAs and elevated lipid ROS.

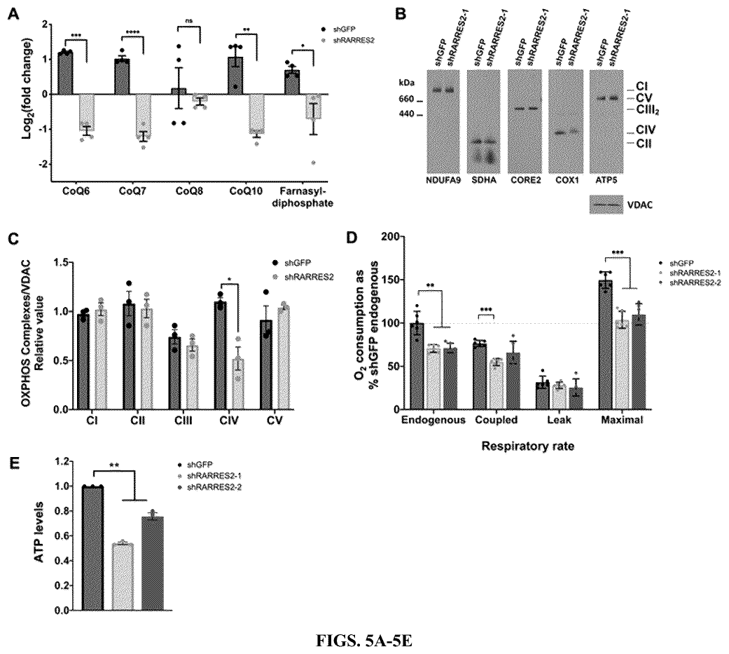

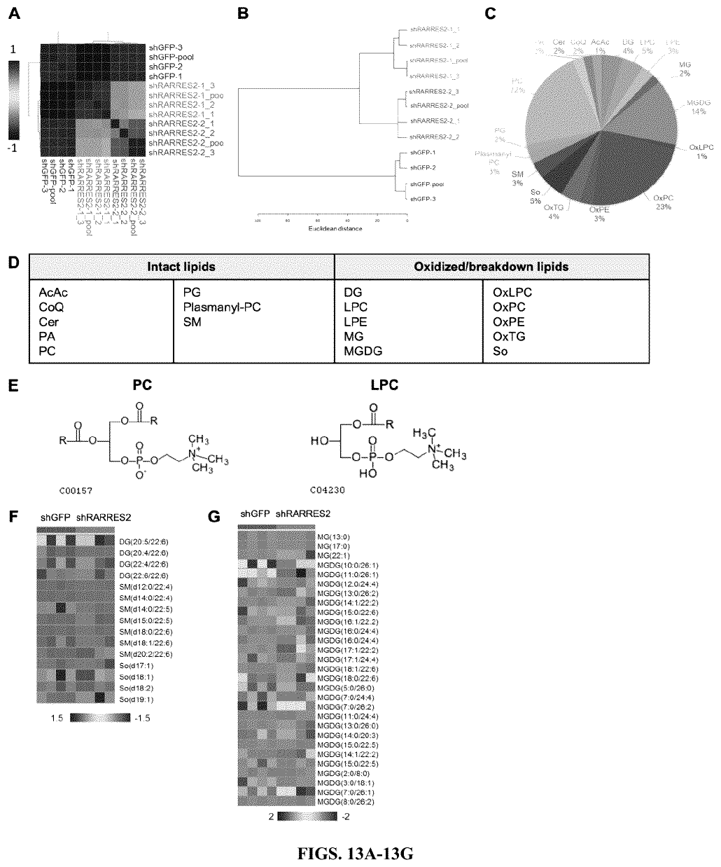

Reduction of OXPHOS complex IV after chemerin inhibition enhances ROS production. As part of the lipid reprogramming in ccRCC cells after chemerin inhibition, this study found that CoQ₁₀, the major form of CoQ found in humans, was also decreased (FIG. 5A). Since CoQ is an electron carrier of the mitochondrial respiratory chain (MRC), this study tested whether chemerin silencing results in mitochondrial dysfunction that can promote ferroptosis. The other components of the MRC and oxidative phosphorylation system (OXPHOS) were analyzed upon chemerin knockdown. The result shows that the level of complex IV (CIV), the fourth and last enzyme of the MRC, was also decreased (FIGS. 5B, 5C, and FIG. 14A). It is relevant to note that CIV contains heme A as a cofactor. Heme A is essential for CIV enzymatic activity and it is also required for CIV biogenesis, since defects in heme A synthesis severely compromise CIV holoenzyme formation. In cells, heme A is present exclusively in CIV and differs from heme B (the more common type of heme in the cell) as it has a farnesyl group at the C2 position of the porphyrin ring. Lipid profiling confirmed that farnesyl-diphosphate, which is the farnesyl group donor for heme A biosynthesis, was reduced after chemerin silencing (FIG. 5A), showing a defect in heme A and, consequently, CIV levels. Furthermore, several phospholipids were shown to intimately interact with CIV and were proposed to play a role in its function and stability. The reduction of phospholipid after chemerin inhibition (FIG. 4F) can also contribute to the observed decrease in CIV.

Due to the alteration in the MRC components CoQ and CIV, this study next sought to investigate whether the mitochondrial respiratory function was affected by chemerin inhibition by polarographic analysis of cellular respiration. Consistently, this study observed a defect in respiratory capacities, including endogenous, coupled and maximal respiration (FIG. 5D), which were similarly observed in Seahorse Mito Stress Test assay (FIG. 14B). This was associated with a reduction in ATP production (FIG. 5E). In order for β-oxidation to support ATP synthesis, cells need a functional MRC so that the electrons from lipid oxidation can be transferred to MRC complexes by reducing equivalents NADH and FADH2. However, chemerin knockdown cells have high FA β-oxidation yet low respiratory capacity. This can create an unbalance between the high electron influx from the β-oxidation pathway and the downstream slow electron flux in the MRC. A large input of electrons in a CIV-compromised MRC represents a favorable condition for the premature electron escape from MRC CI and CIII to molecular oxygen, generating superoxide radicals. The increase in ROS species could in turn contribute to promote ferroptosis (FIG. 4J), consistent with previous observations. It is important also to note that there was no structural change in the filamentous mitochondrial network nor the number of mitochondria after silencing of chemerin (FIG. 14C).

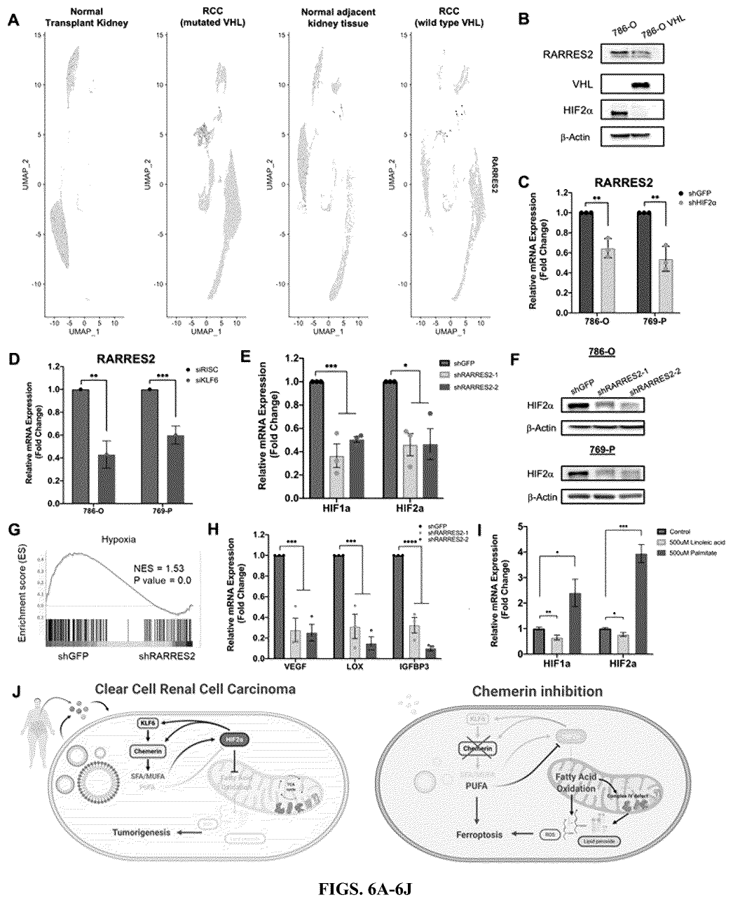

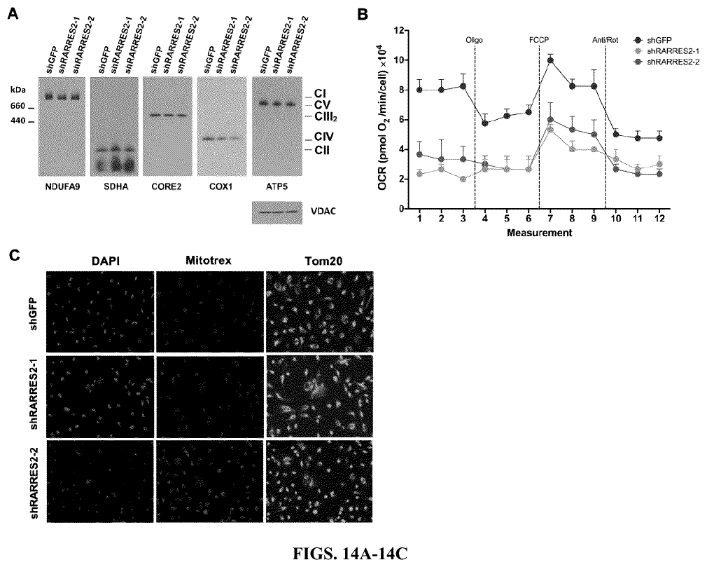

Chemerin expression is VHL dependent and regulates HIF in ccRCC. This study next sought to investigate underlying signaling mechanism of how chemerin regulates fatty acid oxidation and mitochondrial functions. Since most of the ccRCC tumors are VHL null, this study first examined whether chemerin expression is VHL dependent. By interrogating the scRNA-seq data, it was found that chemerin was detected at higher expression only in ccRCC with VHL mutation, but generally not in rare ccRCC that retain wild-type VHL, or in normal kidney epithelium (FIG. 6A, FIG. 15A). To validate this observation, 786-O cells with VHL mutation, were transduced with an adenovirus overexpressing VHL protein (FIG. 6B) to restore VHL function of HIF regulation. When VHL was expressed, this study observed destabilization of HIF2α, and a subsequent reduction in both the chemerin protein as well as mRNA level (FIG. 6B, FIG. 15B). HIF2α silencing also transcriptionally reduced chemerin expression (FIG. 6C, FIG. 15C). Thus, chemerin expression in ccRCC is VHL dependent. In the case of wild type VHL and HIF2α dependent, hypoxia could therefore lead to elevated expression as well.

This study noted from the literature the recent description of the zinc finger DNA-binding transcription factor KLF6 as a target of the VHL-HIF2 axis in ccRCC, and after interrogating the list of KLF6 responsive genes, chemerin was found (FIG. 15D). Accordingly, knockdown of KLF6 leads to reduced chemerin expression (FIG. 6D, FIGS. 15E-15F). Furthermore, RNA-sequencing data of multiple ccRCC cell lines from the Cancer Dependency Map (www.depmap.org), demonstrate a positive correlation between KLF6 and RARRES2 expression (R²=0.8344, p-value=0.0302). (FIG. 15G). Together, the data show that VHL alterations, through deregulation of HIF2α and KLF6, mediates tumor-derived chemerin expression in ccRCC.

Strikingly, several FAO genes in this analysis that were regulated by chemerin have been reported as HIF target genes, and chemerin inhibition reduced transcription of both HIF1α and HIF2α in cells (FIG. 6E). HIF2α protein level was also significantly decreased by chemerin silencing in both 786-O and 769-P cells (FIG. 6F); where HIF1α protein is not detectable. GSEA analysis of the RNA-seq data confirmed that in addition to the lipid pathways noted in FIG. 3D, the hypoxia pathway was one of the most downregulated pathways after chemerin knockdown (FIG. 6G, FIG. 15H). This study validated reduced expression of the HIF target genes VEGF, LOX and IGFBP3 by qRT-PCR (FIG. 6H). As a link between lipid metabolism and HIFα mRNA expression, it was previously demonstrated that the HepG2 cells induce HIF1α expression as a protective measure against fatty acid-induced toxicity. Exogenous addition of palmitate, for example, led to a robust induction of HIF1α mRNA. This study thus tested whether levels of free FA in ccRCC cells could promote HIF1α or HIF2α expression. This study found that palmitate (an SFA), opposite pattern seen in linoleic acid (a PUFA), increases mRNA (FIG. 6I) and protein levels of HIF2α, and subsequently chemerin in ccRCC cells (FIG. 15I), and thus this study shows that through maintenance of low FA metabolism, chemerin participates in a positive feedback loop to maintain high levels of HIF (FIG. 6J).

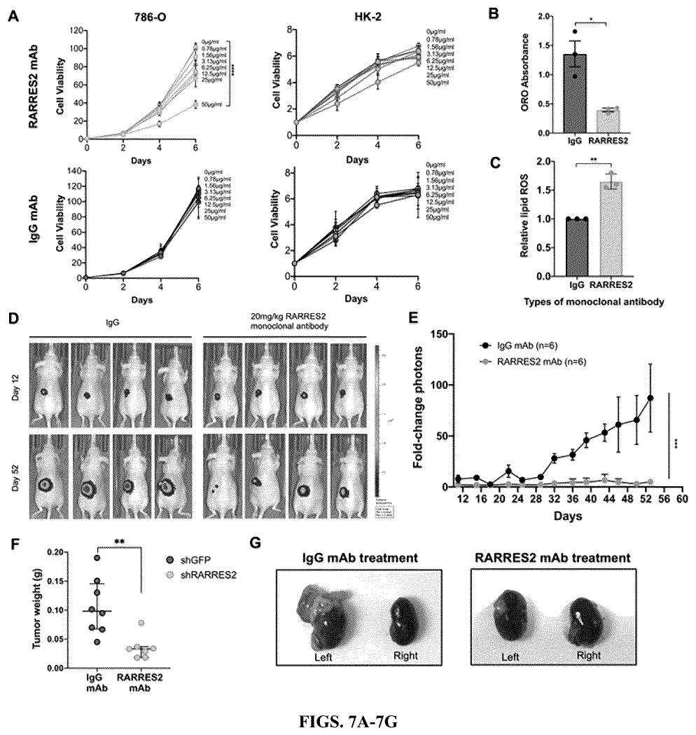

Monoclonal antibody against chemerin reduces ccRCC tumor growth. Given the significant clinical correlates of chemerin in ccRCC, this study investigated the translational impact of inhibiting chemerin with a monoclonal antibody (mAb). Treatment of ccRCC 786-O cells showed a dose-dependent reduction in cell viability with increasing concentrations of chemerin mAb (FIG. 7A, left upper panel). In contrast, cell viability was not affected when HK-2 cells were treated with a similar dose range of the antibody (FIG. 7A, upper right panel). Likewise, neither cell line was affected by control IgG mAb treatment at the same doses (FIG. 7A, lower panels). Importantly, 786-O cells treated with chemerin mAb also showed a decrease in lipid droplets (FIG. 7B) and an increase in lipid ROS (FIG. 7C) as compared to treatment with IgG.

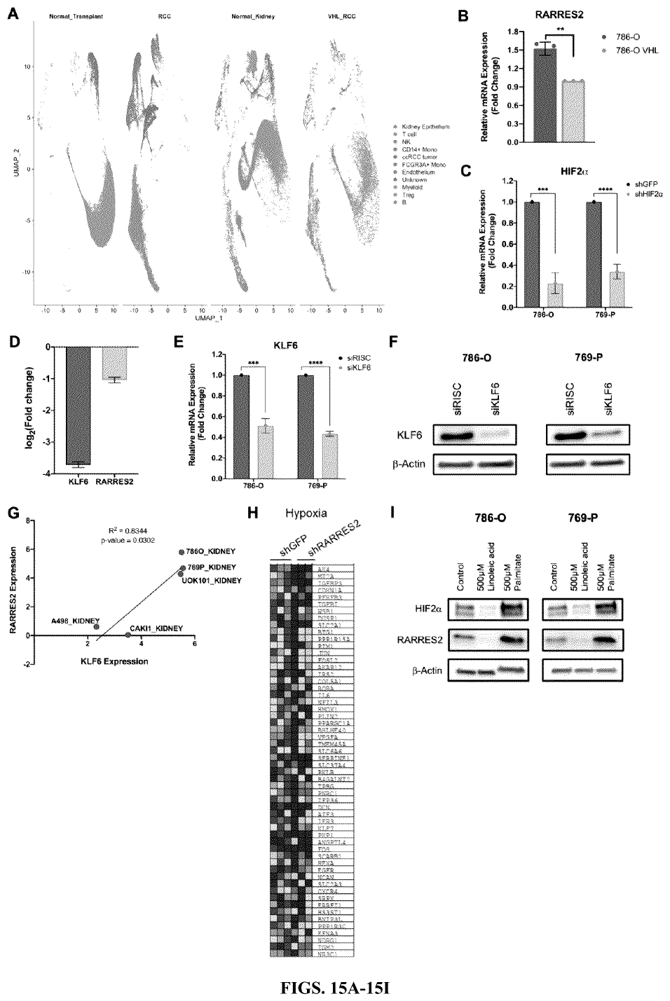

Next, this study examined the potential for therapeutic targeting of chemerin using the antibody in an orthotopic ccRCC model. 786-O cells that were engineered to express firefly luciferase were transplanted under the left kidney capsules in nude mice, and tumor growth was monitored using bioluminescent imaging (BLI). The mice were randomly assigned into two different treatment arms: one receiving 20 mg/kg chemerin mAb twice weekly, and the other receiving IgG control mAb of the same dose. The doses of antibody used in this study did not result in significant changes in animal weight, indicating minimal toxicity (FIG. 16A). Consistent with the cell culture results, in vivo 786-O tumor growth was significantly reduced by treatment of antibody targeting chemerin (FIGS. 7D-7E). The mice treated with chemerin mAb also had lower tumor weights than those receiving IgG mAb upon sacrifice (FIGS. 7F-7G). Collectively, the results show that targeting chemerin therapeutically in ccRCC represents a promising avenue that can have therapeutic effects.

This study utilized a multidisciplinary approach including scRNA-seq, functional genomics, untargeted lipidomics and metabolomics to provide new insights into the role of the adipokine chemerin in lipid metabolism and tumorigenesis in ccRCC. The identification of HIF-dependent chemerin expression to prevent FAO and escape from ferroptosis, highlights a critical metabolic dependency of ccRCC on suppression of lipid metabolism. The findings have several direct implications, including the linkage of an obesity-driven systemic factor with tumor promotion, as well as a novel regulatory mechanism of HIF signaling. The soluble nature of chemerin indicates that both biomarker and therapeutic opportunities can be useful in devising new strategies to treat kidney cancer.

Obesity has been recognized as a global public health issue, and it correlates with higher incidences of cancer. With more than half of the US population currently overweight (BMI more than 25), it is thought that increased body mass is contributing to the rise in kidney cancer incidence. The hypertrophied adipose tissues in obesity can trigger inflammation, hypoxia, and angiogenesis, which subsequently can promote tumor formation. While efforts are underway to elucidate mechanisms explaining the link between obesity and cancer, the dysregulated secretion of signaling molecules by adipose tissue (adipokines), is a culprit. Over the last decade, many reports have demonstrated that adipokines play roles in cancer development. For instance, some adipokines, including leptin, have proinflammatory roles and can stimulate cancer stem cells to become more tumorigenic. Chemerin, a recently discovered multifunctional adipokine, has been shown to regulate adipogenesis, inflammation, angiogenesis and energy metabolism. Chemerin expression increases when preadipocytes differentiate into adipocytes and circulating chemerin has been correlated with BMI of patients. However, the underlying mechanisms of adipokines on cancer pathogenesis and lipid metabolism remain enigmatic. Because normal kidney epithelium undergoes adipogenic trans-differentiation when becoming ccRCC, this study explored the dependency of autocrine and paracrine derived chemerin in lipid metabolism and tumor survival.

While this study found consistently that chemerin expression is elevated at both the mRNA (from database analyses) and at the protein level (from the TMA) in ccRCC as compared to normal adjacent tumors, it is interesting to note that the magnitude of change tended to be less in the protein staining. Considering the extracellular role of chemerin, it is not unreasonable to hypothesize that tumor-derived protein diffuses into the adjacent tissue, as has been shown for VEGF, much like chemerin production from systemic or adjacent fat can diffuse into the tumor or a premalignant lesion and contribute to ccRCC biology. The nature of a secreted factor thus holds promise of both a detectable biomarker and an actionable target pathway. One significant question is relative contributions of systemic chemerin (potentially related to obesity) versus tumor-derived chemerin to ccRCC. These data support a model where obesity elevates chemerin in a supporting manner, that is superseded by tumor production to maintain tumor growth. Considering the levels in the cohort of patients, it appears that approximately 25% of the circulating chemerin could be attributable to obesity, while 75% can be associated with the presence of a tumor. Chemerin is likely not driving tumorigenesis as an oncogene, but rather promoting expansion and survival of an incipient VHL-deficient cell as it rewires its metabolism via constitutive HIF activation. An obesity environment can therefore be tumor promoting.

Critically, although obesity is a recognized as risk factor of ccRCC, it is commonly accepted that higher BMI is associated with improved survival. It remains unclear as to whether this phenomenon, known as the “obesity paradox,” is due to the nature of tumors in obese patients, reverse causation of cancer cachexia, or detection bias. Nevertheless, how chemerin can fit into a model as an obesity-dependent driver of ccRCC and yet be prognostic of worse outcome could be explained by several possibilities. First, it is unlikely that chemerin is the only adipokine playing a role in ccRCC. Indeed, this initial informatics screening process identified other potential proteins that associate with poor outcome and are also overexpressed in tumors. This study did not look for potentially counteracting molecules that can explain better outcomes, but it is possible that they exist. Secondly, these studies have not attempted as of yet to determine how chemerin impacts responses to standard of care approaches such as tyrosine kinase inhibitors. Obesity contributes to tumor formation, but also results in better drug distribution or responses. In fact, there are reports that obesity associates with more highly angiogenic tumors; and chemerin itself has noted roles in recruiting endothelial progenitor cells through a proangiogenic role.

Accumulation of lipids in lipid droplets is a well-established phenomenon in ccRCC, but the significance of lipid storage is still ambiguous. Some have suggested that the significance is to prevent lipotoxicity, while others have proposed a role in suppressing ER stress. The observation that chemerin prevents ferroptosis by suppressing FAO aligns more with the evasion of lipotoxicity theory; but the fact that reduced FAO also supports elevated HIF expression adds a new layer of benefit to ccRCC tumors. Ferroptosis is a recently described form of regulated cell death that results from iron-dependent lipid peroxidation, and is a promising strategy to induce cancer cell death. It was reported that RCC cells are more prone to ferroptosis if they are grown at low cell density (<50% confluency) as compared to high cell density. This can be due to the inadequate production of a secreted factor, such as chemerin, to support cell survival. This study also noted in a prior work that induction of lipid droplets in vitro is density dependent. Thus, the link between ferroptosis evasion and lipid droplet production is the common secretion of chemerin.

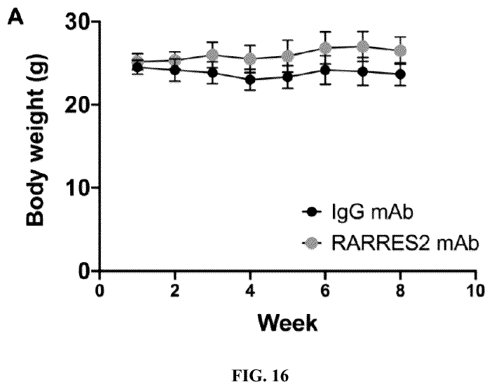

While this current work demonstrated an important role of chemerin in ccRCC tumor biology, the question remains if a receptor is involved for the action of this circulating adipokine. In the literature, chemerin acts as a natural ligand for chemokine-like receptor 1 (CMKLR1, also known as ChemR23), G protein-coupled receptor 1 (GPR1) and C-C chemokine receptor-like 2 (CCRL2). Among these, CMKLR1 is the most well-studied in cancers and chemerin/CMKLR1 has been shown to be involved in gastric cancer, hepatocellular carcinoma metastasis and immune cell trafficking. After knocking out CMKLR1 in 769-P (FIGS. 17A-17B), this study observed a decrease in cell proliferation ability (FIG. 17C) and lipid deposition (FIG. 17D), similar to the effect after chemerin silencing. Some of the lipid metabolism genes regulated by chemerin (CPT1A and PLIN4) were also downregulated by CMKLR1 knockout (FIG. 17E). This shows the involvement of CMKLR1 receptor in lipid biology of chemerin in ccRCC. Yet more work needs to be done to fully elucidate the functional role of CMKLR1 in the ferroptosis pathway. In addition, chemerin binds to GPR1 with similar affinity as CMKLR1 and GPR1 has been demonstrated to be involved in glucose homeostasis in obesity, breast cancer and adipogenesis. On the other hand, CCRL2 is not involved in signal transduction and its role in human biology is still poorly understood. Hence, it is of high interest to clearly delineate the roles of these receptors in ccRCC biology as they can also be useful therapeutic targets.

Chemerin has a physiological role as an inflammatory mediator in homeostasis as well as recently described pathological inflammatory roles in multiple cancers. Thus, the effect of chemerin on tumor growth can also be driven by its function on inflammatory cells. Chemerin has been reported to recruit immune effector cells and increase T-cell mediated cytotoxicity in breast cancer, prostate cancer and sarcoma models. However, these cancers, in contrast to ccRCC, have lower chemerin expression in tumor tissues than healthy individuals, and higher chemerin expression is correlated with better survival outcome. It was demonstrated in multiple reports that chemerin increases NK cell and CD8+ T cell infiltration in orthotopic syngeneic models, thus these tumors downregulate chemerin expression as a means of immune escape. On the other hand, chemerin has also been shown to promote Treg differentiation, and promotes polarization of M2 macrophages in pRCC, both of which are tumor-promoting. It was also noted that chemerin plays a role in increasing lipid deposition in macrophages, showing the interplay of chemerin in immune cell modulation, lipid deposition and tumor progression. In the study of pRCC, elevated chemerin did associate with worse survival.

The classic triad symptoms of RCC are hematuria, abdominal pain and a palpable mass, yet most patients are asymptomatic until incidental detection by radiographic imaging. Most of the cases therefore have advanced to higher stages or even metastasized when diagnosed. Hence, there is a need to have a circulating biomarker that can be useful for early detection. Several adipokines have been described as possible biomarker candidates. For instance, a large case-control study in 2013 reported reduced serum adiponectin in RCC patients, but one of the biggest limitations of the study is that only males were included. Moreover, the correlations of adipokines (including adiponectin and leptin) and RCC risks are inconsistent in multiple reports, further weakening their potential as biomarkers. Serum chemerin is higher in oral squamous cell carcinoma patients than healthy individuals. In the current report, this study detected an elevated chemerin level in ccRCC patient plasma, which is also correlated with BMI status. Nonetheless, plasma chemerin can be informative when combined with existing practice tools for early detection and follow up ccRCC patients.

The implications of an adipokine regulating lipid metabolism and ferroptosis in ccRCC can have broad impact, as obesity is increasingly recognized as risk factor for a multitude of cancers. Before the recent advances in immunotherapy, the primary focus of cancer therapy had traditionally been on tumor and tumor microenvironmental phenotypes. Consideration of systemic mediators of transformation and progression has remained an overlooked attribute. While obesity driven metabolic mediators can be particularly relevant for highly lipid-laden cancers such as ccRCC and clear cell ovarian tumors, metabolic alterations in cancers are essentially universal. Targeting chemerin to block its downstream effects on fatty acid oxidation (FAO) and ferroptosis is therefore a novel therapeutic approach.

### Example 3. Methods

The example shown here combines both experimental and computational efforts. In particular, a variety of techniques are utilized, bringing together microfluidics, next-generation sequencing, and protein science technologies, combined with computational analysis of the experimentally generated datasets.

Cell Cultures. 786-O, UOK101, A498, and HEK293T cells were cultured in DMEM (Corning, Cat #10-013-CV) containing 10% fetal bovine serum (FBS) (VWR, Cat #89510-186). 769-P was cultured in RPMI (Gibco, Cat #11875-093, 31800-022) containing 10% fetal bovine serum. HK-2 cell was cultured in Keratinocyte Serum Free Medium (K-SFM) (Invitrogen, Cat #10724-011) supplemented with bovine pituitary extract (BPE) and human recombinant epidermal growth factor (EGF). 769-P, A-498, HEK293T and HK-2 were obtained from ATCC. 786-O and UOK-101 were gifts of Dr. Sandra Turcotte and Dr. W. Marston Linehan (National Cancer Institute, Bethesda), respectively. Cells were used for experiments within 10-15 passages from thawing. All the cells were tested to ensure they are mycoplasma free and they were authenticated by short tandem repeat profiling by Genetica DNA Laboratories.

Animal Studies. All animal experiments were in compliance with National Institutes of Health guidelines and were approved by the Institutional Animal Care and Use Committee (IACUC) of the University of Miami Miller School of Medicine. Six-week-old female athymic nude mice (Charles River Laboratories) were used for xenograft studies. Mice were housed (3-5 mice per cage) on a 12-hour light/dark cycle in individually ventilated cages, and with ad libitum access to food and water. The experimental holding room had temperature (21.5° C. set point) and humidity control (40% set point), and it was equipped with HEPA-filtered air. For subcutaneous tumor growth model, cells were pelleted and resuspended in a PBS/Matrigel Matrix (Corning, Cat #356234) mix at 1:1 ratio. 2×10⁶ cells in a 100 μL solution were injected subcutaneously into each flank. Once palpable tumors were established, digital calipers were used to measure tumor size twice weekly. Tumor volume was calculated using the equation V=½ (length×width²). For orthotopic tumor growth assays, soft collagen pellets containing 1×106 cells were implanted orthotopically under the capsules of the left kidney. Tumors were palpated and monitored twice weekly using IVIS bioluminescence imaging (Perkin Elmer). At the end of the experiments, blood was collected and both kidneys were harvested.

Patient Information. Fresh normal-tumor tissues were obtained from nephrectomy cases at the University of Miami Hospital. Patients' whole blood was also obtained and processed in the laboratory to obtain plasma. The samples were obtained under approvals from institutional research ethics review committees and with written patient consent under the institutional review board protocol 20140385.

RNA Isolation and Real Time Quantitative Polymerase Chain Reaction (qRT-PCR). Total RNA was extracted using TRIzol reagent (Invitrogen, Cat #15596018). The concentration and quality of RNA were determined using a NanoDrop 1000 spectrophotometer (Thermo). 500 ng of RNA was used to synthesize cDNA using the Quantabio qScript cDNA SuperMix (Quantabio, Cat #95048-100) according to the manufacturer's recommendations. The cDNA was diluted by 10-fold before used for qRT-PCR gene expression analysis. qRT-PCR was performed using Power SYBR Green PCR Master Mix (Applied Biosystems, Cat #4367659) and SYBR Green primers (Table 3). The Ct values of the gene of interest was normalized to the Ct value of the housekeeping control, β-actin. The fold change was then calculated using the 2^{-ΔΔCt} method.

Lentiviral transduction. For shRNA knockdown models, HEK293T cells were transfected with a mixture of the lentiviral transfer plasmid of interest (Table 4), psPAX2 and PVSVG using Lipofectamine 2000 transfection reagent (Invitrogen, Cat #11668-019). shRNA GFP was used as a control. The media containing the viral particles was collected 24 hours post-transfection and filtered through 0.45 μm PVDF sterile syringe filter (Santa Cruz). Cells were infected with the lentiviral supernatant in the presence of 10 μg/mL of polybrene (Sigma-Aldrich, Cat #H9268). RARRES2 knockout cell lines were generated by cloning sgRNAs (Table 5) into the lentiviral vector lentiCrisprv2 (Addgene), followed by lentivirus production in HEK293T. After puromycin selection, single cell clones were generated by limiting dilution in 96 well plates. Single-cell clones were expanded and lysates were collected to verify knockout by western blotting.

Protein Extraction and Western blotting. The cells were washed with 1× ice-cold PBS (GE, Cat #SH30256.01) once before they were lysed in RIPA lysis buffer (50 mM Tris-HCl, pH 7.5; 150 mM NaCl; 0.5% NP-40; 50 mM NaF) and scraped from the plate on ice. The RIPA lysis buffer contained 1:1000 1× protease inhibitor cocktail (Sigma-Aldrich) and 1:100 1× phosphatase inhibitor (Sigma-Aldrich). The lysates were passed through a 16G needle 15 times before they were centrifuged at 4° C. for 20 minutes at 20,000×g. The protein lysates were quantified using the Pierce BCA Protein Assay Kit (Thermo) according to the manufacturer's protocols and absorbance was measured using GloMax Discover Microplate Reader (Promega). Between 50 and 100 μg of protein samples were mixed with 5×SDS Laemmli loading buffer and boiled at 95° C. for 5 minutes. Proteins were separated using 4-20% SDS-PAGE gels (Bio-Rad) and transferred onto PVDF membrane (Millipore). The membrane was blocked with TBS-T supplemented with 5% non-fat dry milk primary antibody for 45 minutes at room temperature rocking before blotting with primary antibodies at 4° C. overnight. The membrane was washed in TB S-T 3 times with 5 minutes each and blotted in secondary antibodies at room temperature for 2 hours. An Azure 300 chemiluminescent western blot imaging system (Azure biosystems) was used to visualize the membrane. The antibodies used were listed in Supplementary Table S6.

Enzyme-linked Immunosorbent Assay (ELISA) for Plasma Chemerin. Plasma were diluted 1:1000 before incubated in a high-binding microplate (Santa Cruz) coated with capture antibody according to manufacturer's instructions (R&D, Cat #DY2324). The samples were washed, and biotin-conjugated detection antibody was added into the plate wells for further incubation. Next, HRP-conjugated streptavidin was added, followed by substrate AB 1:1 mixture. Finally, the catalytic reaction was terminated by adding the stop solution and the absorbance was measured at 450 nm using GloMax Discover Microplate Reader (Promega).

Cell Proliferation Assay. Cells were plated at a density of 100,000 cells in triplicate wells of a 6-well plate, supplemented with DMEM containing 10% FBS. Cells were trypsinized and were counted using the Cell Counter Countess II (Life Technologies) at days 3, 6, 9 and 12.

Cell Viability Assay. After treatment with monoclonal antibodies, 786-O cells were incubated with 3-(4,5-dimethylthiazol-2-yl)-2,5-diphenylterazolium bromide (MTT, Sigma-Aldrich) to assess cell survival. Briefly, the cells were plated at density of 1000 cells/plate in 96-well plates and treated with different concentrations of monoclonal antibodies (IgG or anti-RARRES2) for indicated incubation times (2 days, 4 days and 6 days). MTT solution was diluted 5-fold in DMEM media and incubated in 37° C. incubator for 4 hours. The media was carefully removed and the precipitate was dissolved in DMSO. The solution later immediately quantified at 450 nm using the GloMax Discover Microplate Reader (Promega).

EdU incorporation assay. Cells were plated on coverslips at 1×10⁴ cells in 12-well plate and incubated with 10 μM EdU solution overnight at 37° C., 5% CO₂. After incubation, the cells were fixed with 3.7% formaldehyde/PBS for 15 minutes at room temperature and washed with 1 mL of 3% BSA/PBS twice. 1 mL of 0.5% TritonX-100 in PBS was added for 20 minutes at room temperature to permeabilize the cells. Next, cells were incubated with 0.5 mL of Click-iT reaction cocktail (Invitrogen, Cat #C10337) prepared according to manufacturer's instructions, at room temperature for 30 minutes and away from direct light. DAPI (Millipore, Cat #268298) was added later for DNA staining. Coverslips were removed from wells and visualized using the z-stack of an Olympus FV1000 confocal scanning microscope. The images were analyzed using Fiji software (ImageJ).

Clonogenic survival assay. The cells were plated in triplicate in 6 cm plates (100 cells/well) with complete DMEM media to measure survival by colony forming unit (CFU). The colonies were counted after 2 weeks and the experiment was repeated twice.

siRNA transfection. In one tube, the stock siRNA was diluted in serum-free medium and DharmaFECT transfection reagent (Horizon) was added to another tube. The content of each tube was gently mixed and incubated for 5 minutes at room temperature. The contents of both tubes were mixed and incubated for 20 minutes at room temperature. The culture media were removed from the plate and 2.5 mL of serum-free medium was added together with the transfection medium. The cells were incubated at 37° C. in 5% CO2 for 5 hours before changing the media to complete media with 10% FBS. Non-targeting Control siRNAs (siRISC) and KLF6 targeting siRNA (Horizon) were used.

Flow Cytometry. Cells were stained with fluorescein isothiocyanate (FITC)-conjugated annexin V and propidium iodide (ChemCruz, Cat #sc-3541) using the Alexa Fluor 488 Annexin V Kit (Invitrogen, Cat #V13245) according to the manufacturer's protocol. The cells were later analyzed using an LSR II cytometer instrument (BD Biosciences) and analyzed by FACS DIVA 8.1 software (BD Biosciences). The percentage of dead cells in measured by combining cells with annexin V-positive and annexin V/PI-double-positive.

BODIPY Lipid Droplet Imaging. Cells were plated on round glass coverslips of 12-well plates and incubated with 1 mL of DMEM supplemented with 10% FBS. The medium was removed and cells were washed with room temperature PBS, fixed with 4% formaldehyde for 30 minutes and incubated with 300 μL of 1 uM BODIPY 493/503 (Life Technologies, Cat #D3922) for 15 minutes, protected from direct light. 1 μg/mL of DAPI (Millipore) was added for nucleus staining. The z stack images were acquired using Olympus FV1000 confocal scanning microscope and analyzed using Fiji software (ImageJ).

Lipid Reactive Oxygen Species. The cells were plated at 50,000 cells per well in 24-well plate. 10 uM BODIPY-581/591 C11 (Invitrogen, Cat #D3861) and 1 ug/mL DAPI (Millipore) were added to the media and incubated for 1 hour at 37° C., 5% CO₂. Cells were imaged using Olympus FV1000 confocal scanning microscope and analyzed using Fiji software (ImageJ). 9 randomly selected images per condition were selected for analysis.

Oil Red O (ORO) Lipid Droplet Staining. For tissue sections staining, the tissues were cryoprotected by incubating in 30% sucrose/PBS solution. After the tissues sank to the bottom of the vial, the tissues were embedded in OCT (Fisher Healthcare). Embedded tissue blocks were sectioned at 10 μm thickness and stored at −80° C. until ready for staining. Before ORO staining, the frozen sections were air dried and fixed in 10% neutral buffered formalin (Sigma-Aldrich, Cat #HT501128). After fixing in 100% propylene glycol (VWR, Cat #0575) twice, sections were stained with freshly made ORO solution (Sigma-Aldrich, Cat #00625) followed by staining for hematoxylin. Then, the slides were washed with distilled water and mounted using glycerin jelly (Sigma-Aldrich). The sections were visualized using a VS120 Virtual Slide Microscope scanner (Olympus). For cell staining, the cells plated in 12-well plates in triplicate were rinsed with PBS twice, and fixed with 10% formaldehyde for 1 hour. They were then rinsed with 60% isopropanol for 5 minutes, followed by ORO staining (3 mg/mL) for 4 minutes and the ORO solution was removed. After the cells were air dried, 250 μL of isopropanol was added to the wells and the eluted solution was measured at 510 nm. Next, the cells were stained with sulforhodamine B as previously described, and measured at 564 nm using GloMax Discover Microplate Reader (Promega), as previously described.

BN-PAGE analysis. Blue native-polyacrylamide gel electrophoresis (BN-PAGE) was performed as previously described. Briefly, 2×10⁶ shGFP control and shRARRES2 cells were collected, resuspended in PBS and permeabilized with 2 mg/mL digitonin. Permeabilized cells were pelleted and resuspended in 100 μl buffer containing 1.5 M aminocaproic acid, 50 mM Bis-Tris pH 7.0. Mitochondrial proteins were extracted in the presence of 1% lauryl maltoside (LM), followed by clarification spin at 22,000 g for 30 minutes at 4° C. Native PAGE™ Novex® 3-12% Bis-Tris protein gels (Life Technologies) were loaded with 60-80 μg of total LM cell extracts. After electrophoresis, proteins were transferred to PDVF membranes and used for immunoblotting. Western blot was performed using primary antibodies raised against the following human OXPHOS subunits: NDUFA9 (Abcam, ab14713), CORE2 (UQCRC2, Abcam, ab14745), COX1 (Abeam, ab14705), SDHA (Protein tech, 14865-1-AP), ATP5 (ATP5A, Abcam, ab14748). In parallel, 60 μg aliquots of the same samples were resolved in a denaturing 12% SDS-PAGE and probed with a primary antibody against VDAC as loading control. Peroxidase-conjugated anti-mouse and anti-rabbit IgGs were used as a secondary antibodies (Rockland).

Polarographic analysis of cellular respiration. Endogenous cell respiration was measured polarographically at 37° C. using a Clark-type electrode from Hansatech Instruments (Norfolk, United Kingdom). Substrate-driven respiration was assayed in digitonin-permeabilized cultured cells as reported. Briefly, trypsinized cells were washed with permeabilized-cell respiration buffer (PRB) containing 0.3 M mannitol, 10 mM KCl, 5 mM MgCl₂, 0.5 mM EDTA, 0.5 mM EGTA, 1 mg/ml BSA and 10 mM KH₃PO₄ (pH 7.4). The cells were resuspended at −4×10⁶ cells/ml in 0.5 ml of the same buffer air-equilibrated at 37° C. and supplemented with 10 units of hexokinase and 2 mM ADP. The cell suspension was immediately placed into the polarographic chamber to measure endogenous respiration. Cells were then permeabilized with digitonin (3.4 μg/10⁶ cells) and complex I substrates glutamate and malate (to 2 mM each final concentration) were added to measure state 3 or ADP-stimulated (coupled) respiration. Oligomycin (to 1 μM final concentration) was successively added to inhibit ATP synthesis, and non-phosphorylating condition (state 4 or leak respiration) was measured. Mitochondrial respiration was uncoupled by 1 μL incremental addition of 0.1 M CCCP to reach maximal oxygen consumption. Lastly 3 μL of an 80 mM KCN solution were finally added to assess the specificity of the assay. Oxygen consumption rate were measured as nmol O₂ consumed/min, normalized by number of cells and expressed as percentage of the shGFP control endogenous respiration.

ATP Measurement. The relative cellular ATP concentration was measured using Cell Titer-Glo Luminescent reagent (Promega) according to manufacturer's protocol. Briefly, an equal volume of the single-one-step reagent was added to each well of cells and incubated at room temperature for 15 minutes rocking. The luminescence was quantified using GloMax Discover Microplate Reader (Promega). The ATP level is normalized to cell count from an additional plate with same setup.

Extracellular Flux Analysis. Prior to the Seahorse XF96 Cell Mito Stress Test assay, the optimal cell density was determined to be 2000 cells per well. A day before the experiment, the cells were plated in the Seahorse XF96 Cell Culture Microplate (Agilent 102601-100) using DMEM. The sensor cartridge was hydrated in Seahorse XF Calibrant at 37° C. in a non-CO₂ incubator overnight. The medium (Agilent 103575-100) was supplemented with 1 mM pyruvate, 2 mM glutamine and 10 mM glucose before adjusting the pH to be 7.4. 20 μL of 15 μM stock Oligomycin, 22 μL of 5 μM stock FCCP and 25 μL of 5 μM stock Rotenone/Antimycin A, were added to Port A, B and C of the microplate respectively. After replacing the cell media in the cell culture microplate with warmed assay medium using a multichannel pipette, the microplate was placed in a 37° C. non-CO₂ incubator for 45 hours. The assay was run using the Agilent Seahorse XF96 Analyzer (Seahorse Biosciences) and the assay parameters (including oxygen consumption rate) were exported from the Seahorse XF Stress Test Report Generator.

Immunohistochemistry staining. The paraffin-embedded ccRCC tumor microarray (KD601) was purchased from US Biolab that contains clear cell renal cell carcinoma tissue with matched cancer adjacent kidney tissue, 2 cores per case. The slides were baked for 60 min at 60° C., deparaffinized in xylene, and rehydrated through graded concentrations of ethanol in water. The slides were then subjected to antigen retrieval. Immunohistochemistry staining was performed using BOND-III Fully Automated IHC and ISH Stainer according to the manufacturer's instructions (Leica Biosystems, Wetzlar, Germany). Anti-rabbit polyclonal human chemerin (29-67) (H-002-52, Phoenix Pharmaceuticals) was used at 1:200 dilution, and other reagents for IHC were provided by Bond™ Polymer Refine Detection (DS9800) kit. The microarray sections were left to air-dry, mounted with permanent mounting medium and coverslipped. The bright field images were acquired by an automated VS200 Research Slide Scanner (Olympus) at 40× magnification. The images were subsequently viewed using the OlyVIA software (Olympus). The intensity of the staining was quantified using Image J.

Sodium palmitate preparation and supplementation. To prepare BSA-palmitate conjugate, sodium palmitate (Thermo, Cat #P0500) was added to a NaCl solution and heated in a water bath while stirring. The hot palmitate solution was mixed with BSA solution, 5 mL at a time. The final volume was adjusted with 150 mM NaCl for 1 mM palmitate solution and pH was adjusted to 7.4. Sodium palmitate solution was added to cell at final concentration 500 μM and incubated for 24 hours.

RNA-sequencing. Total RNA was extracted using the RNeasy Mini Kit (Qiagen, Cat #74104) according to the manufacturer's instructions. Before library construction, all samples were assessed for RNA purity via 260/280 ratio using Nanodrop, then RNA integrity and potential DNA contamination using agarose gel electrophoresis, and RNA integrity again using the Agilent Bioanalyzer 2100. mRNA was purified from total RNA using poly-T-oligo-attached magnetic beads and the mRNA were fragmented randomly by addition of fragmentation buffer. For NEB library preparation using NEBNext® Ultra® RNA Library Prep Kit for Illumina®, briefly, the first strand cDNA was synthesized using random hexamer primer and M-MuLV Reverse Transcriptase (RNase H-) and second strand cDNA synthesis was subsequently performed using DNA polymerase I and RNase H. The double-stranded cDNA was then purified using AMPure XP beads and the remaining overhangs of the purified double-stranded cDNA are converted into blunt ends via exonuclease/polymerase activities. After adenylation of 3′ ends of DNA fragments, NEBNext Adaptor with hairpin loop structure was ligated to prepare for hybridization. In order to select cDNA fragments of preferentially 150200 bp in length, the library fragments are purified with AMPure XP system (Beckman Coulter, Beverly, USA). Finally, the final library was obtained by PCR amplification and purification of PCR products by AMPure XP beads. The libraries were pooled in equimolar amounts and sequenced in paired end 150 bp reactions on the Illumina NovaSeq 6000.

Untargeted lipidomics and metabolomics. Cell samples were extracted using the modified folch biphasic extraction procedure. Briefly, for metabolomics, 20 μL of internal standard mixture were added to cell pellets of 10 million cells. Protein quantification were done for all the samples for pre-normalization. Ice cold methanol (80%) were used for global metabolome extraction. The supernatant was collected after centrifugation, transferred to a new tube and dried under nitrogen. The dried sample was reconstituted in 0.1% formic acid in water (70 For lipidomics, 20 μL of 10× diluted internal standard mixture (stock solution of 50 ppm, w:v) was added. Samples were extracted using ice cold 4:2:1 chloroform:methanol:water (v:v:v), and the organic phase was collected, dried down under nitrogen flow, and reconstituted in 75 μL of isopropanol plus 1 μL of injection standard mixture (100 ppm, w:v). They were used for global lipidome extraction. Metabolomics and lipidomics samples ran separately and for each sequence, solvent blanks, extraction blanks (without internal standard), neat quality controls (QCs, blanks with internal standards), pooled samples QCs were also prepared for evaluation of extraction and data collection efficiency. High-pressure liquid chromatography coupled high-resolution tandem mass spectrometry (LC-HRMS/MS) were used for data collection. Chromatographic separation was achieved using reverse phase chromatography (Dionex Ultimate 3000 RS UHLPC system, Thermo Scientific, San Jose, CA, USA) with a Waters C18-pfp column (Ace, 100×2.1 mm, 2 μM) for metabolomics and with a Waters Acquity C18 BEH column maintained at 30° C. (2.1×100 mm, 1.7 μm particle size, Waters, Milford, MA, US) for lipidomics. In the case of metabolomics, the gradient consisted of solvent A (0.1% FA in H2O) and solvent B (Acetonitrile), both with 10 mM ammonium formate and 0.1% formic acid. The flow rate was 350 μL/min. The column temperature was maintained at 25° C. In the case of lipidomics, the gradient consisted of solvent A (60:40 acetonitrile:water) and solvent B (90:8:2 isopropanol:acetonitrile:water), both with 10 mM ammonium formate and 0.1% formic acid. The flow rate was 500 μL/min. The column temperature was maintained at 50° C. Samples were analyzed in positive and negative electrospray ionization on a ThermoScientific Q-Exactive mass spectrometry (Thermo Scientific, San Jose, CA). Data-dependent (ddMS2-top5) MS/MS data were obtained on pooled samples per group for identification purposes. In addition, full-scan data was acquired for all the samples without MS/MS for comparing metabolite or lipid intensities across groups.

Quantification and statistical analysis. Unless indicated in the figure legend, all experiments were performed at least in triplicate and results were presented as mean±standard error of mean (SEM) of absolute values or percentages of control. All the statistical analyses performed were described in each figure legend. Statistical p values were obtained by application of the appropriate statistical tests using the Prism 8 program. P-values lower than 0.05 were considered significant (*p<0.05; **p<0.01; ***p<0.001; ****p<0.0001; ns, not significance).

RNA-sequencing data analysis. The raw FASTQ data were processed through an in-house bioinformatics pipeline including quality and adapter trimming with TrimGalore version 0.6.1 (www.bioinformatics.babraham.ac.uk/projects/trim_galore/), alignment to the human genome hg38/GRCh38 and gene quantification with the STAR algorithm v2.5.2. The differential expression analysis was performed using the quasi-likelihood function implemented in edgeR. Gene set enrichment analysis was performed using fully unfiltered gene list in the GSEA desktop application (software.broadinstitute.org/gsea/downloads.jsp).

Single cell RNA-sequencing data analysis. Single-cell RNA-sequencing data was assessed from Young et al. and analyzed as previously described. Briefly, in order to remove low quality cells, only single cell libraries with at least 1000 features with greater than 1 count per million were retained and cells with high fraction of counts from mitochondrial genes (indicating dying cells) were removed. In addition, genes with expression less than 1 count per million in at least 2 cells per tumor were excluded from analysis. After concatenating cell by gene count matrices of all samples into a single matrix and normalized, the relationship between cells was visualized in two dimensions through Uniform Manifold Approximation and Projection (UMAP) plots. Each cluster was assigned to respective cell identity by marker differential gene expressions described in previous literatures. The data supplement from Young et al. defined tissue of origin, and was used to stratify during several different analyses. The analysis followed the primary workflow of the Seurat pipeline. According to the authors, the data was normalized by dividing the total number of unique molecular identifiers (UMIs) in each cell to obtain the sequencing depth before transforming to a log scale for each cell using the Seurat (http://satijalab.org/seurat/) NormalizeData function:

$${y}_{gc} = log ( 1 + F \frac{{x}_{gc}}{{\sum }_{g} {x}_{gc}} )$$

x: UMI count matrix
g: gene
c: cell
F: Seurat “scale.factor” (default value 10,000)

Next, COMBAT was used in batch correction step to regress out the variability introduced by individual 10× channels. All the entries which were 0 before batch correction were maintained to be at 0 after batch correction to prevent imputing expression from genes with no expression. The data was re-normalized so that it was consistent with being derived from an expression vector with the sum to 1 after such correction:

$${\sum }_{g} ( {e}^{{y}_{gc}} - 1 ) = F$$

The threshold for mitochondrial genes is set at 5%.

Lipidomics and metabolomics data analysis. For lipidomics data analysis, LipidMatch Flow was used for file conversion, peak picking (implementing MZMine 2, blank filtration, lipid annotation, and combining positive and negative datasets. LipidMatch Flow software and tutorials (including video tutorials) can be found at secim.ufl.edu/secim-tools/. LipidMatch was used to annotate ions using data dependent MS/MS analysis. For metabolomics data analysis, metabolite identification was performed with MZmine 2.0 and matching metabolite retention time and m/z value to an internal library of over 1000 metabolites. Metabolic pathway analysis was conducted using the Kyoto Encyclopedia of Genes and Genomes (KEGG) pathway database by matching metabolite sets with human metabolome (www.genome.jp/kegg/pathway.html). Metabolite set enrichment (fold enrichment) were investigated using MetaboAnalyst (open source R package). The RNA-sequencing data was deposited in GEO database GSE159716. The lipidomics and metabolomics data were deposited in MetaboLights MTBL2435.

TABLE 1
Differential expression matrix utilized in single-cell RNA sequencing
data analysis to identify cell clusters, Related to FIG. 1.

| | p_val | avg_logFC | pct.1 | pct.2 | p_val_adj | cluster | gene |
|---|---|---|---|---|---|---|---|
| 1 | 2.33E−13 | 1.13706469 | 1 | 0.369 | 5.53E−09 | 0 | SMIM24 |
| 2 | 2.09E−12 | 1.2526384 | 1 | 0.356 | 4.95E−08 | 0 | GSTA1 |
| 3 | 9.16E−12 | 1.14127977 | 1 | 0.371 | 2.17E−07 | 0 | NAT8 |
| 4 | 5.82E−11 | 1.32278455 | 0.842 | 0.314 | 1.38E−06 | 0 | PRAP1 |
| 5 | 5.86E−11 | 1.25721015 | 0.842 | 0.268 | 1.39E−06 | 0 | GSTA2 |
| 6 | 2.16E−10 | 1.18964875 | 1 | 0.502 | 5.12E−06 | 0 | FXYD2 |
| 7 | 3.19E−10 | 1.5495097 | 1 | 0.653 | 7.56E−06 | 0 | GPX3 |
| 8 | 5.67E−10 | 1.41319349 | 0.895 | 0.383 | 1.34E−05 | 0 | UGT2B7 |
| 9 | 9.18E−10 | 1.18238298 | 0.789 | 0.268 | 2.18E−05 | 0 | DPEP1 |
| 10 | 1.61E−09 | 1.82701734 | 0.947 | 0.445 | 3.82E−05 | 0 | SPP1 |
| 11 | 0 | 1.29251681 | 0.867 | 0.194 | 0 | 1 | RGS1 |
| 12 | 0 | 1.27547813 | 0.981 | 0.451 | 0 | 1 | BTG1 |
| 13 | 0 | 1.27334569 | 0.787 | 0.184 | 0 | 1 | CCL5 |
| 14 | 0 | 1.24743324 | 0.944 | 0.298 | 0 | 1 | CXCR4 |
| 15 | 0 | 1.2395727 | 0.614 | 0.059 | 0 | 1 | GZMK |
| 16 | 0 | 1.22534593 | 1 | 0.736 | 0 | 1 | TMSB4X |
| 17 | 0 | 1.13419167 | 0.866 | 0.278 | 0 | 1 | ZFP36L2 |
| 18 | 0 | 1.0380251 | 1 | 0.897 | 0 | 1 | B2M |
| 19 | 0 | 0.97783515 | 0.757 | 0.159 | 0 | 1 | CD69 |
| 20 | 0 | 0.97104098 | 0.999 | 0.964 | 0 | 1 | RPS27 |
| 21 | 1.28E−86 | 1.11996081 | 1 | 0.499 | 3.03E−82 | 2 | FXYD2 |
| 22 | 5.26E−82 | 1.05296776 | 0.874 | 0.354 | 1.25E−77 | 2 | GSTA1 |
| 23 | 1.93E−81 | 1.2157664 | 1 | 0.652 | 4.58E−77 | 2 | GPX3 |
| 24 | 2.16E−68 | 0.98685794 | 0.747 | 0.266 | 5.12E−64 | 2 | GSTA2 |
| 25 | 9.17E−67 | 0.91516763 | 0.864 | 0.381 | 2.17E−62 | 2 | UGT2B7 |
| 26 | 3.78E−66 | 0.93675198 | 0.99 | 0.563 | 8.96E−62 | 2 | ALDOB |
| 27 | 1.50E−58 | 0.84946764 | 0.742 | 0.312 | 3.56E−54 | 2 | PRAP1 |
| 28 | 7.59E−56 | 0.79670525 | 1 | 0.964 | 1.80E−51 | 2 | FTL |
| 29 | 1.93E−51 | 0.78471767 | 0.894 | 0.443 | 4.57E−47 | 2 | SPP1 |
| 30 | 5.62E−44 | 0.88269511 | 0.783 | 0.411 | 1.33E−39 | 2 | CRYAB |
| 31 | 0 | 1.75769636 | 0.99 | 0.259 | 0 | 3 | MTRNR2L8 |
| 32 | 0 | 1.38247461 | 1 | 0.978 | 0 | 3 | MT-CO3 |
| 33 | 0 | 1.34784555 | 1 | 0.959 | 0 | 3 | MT-ND3 |
| 34 | 0 | 1.34551515 | 1 | 0.974 | 0 | 3 | MT-ATP6 |
| 35 | 0 | 1.34518217 | 1 | 0.978 | 0 | 3 | MT-ND4 |
| 36 | 0 | 1.28424027 | 1 | 0.959 | 0 | 3 | MT-CYB |
| 37 | 0 | 1.27180382 | 1 | 0.977 | 0 | 3 | MT-CO2 |
| 38 | 0 | 1.24880372 | 1 | 0.986 | 0 | 3 | MT-CO1 |
| 39 | 0 | 1.23924522 | 1 | 0.944 | 0 | 3 | MT-ND1 |
| 40 | 0 | 1.22551173 | 0.931 | 0.216 | 0 | 3 | MTRNR2L12 |
| 41 | 0 | 1.50621274 | 0.983 | 0.489 | 0 | 4 | MT1G |
| 42 | 0 | 1.29085758 | 0.932 | 0.369 | 0 | 4 | MIOX |
| 43 | 0 | 1.21682324 | 0.99 | 0.513 | 0 | 4 | ALDOB |
| 44 | 0 | 1.13682999 | 0.825 | 0.319 | 0 | 4 | MT1H |
| 45 | 0 | 1.11862845 | 0.929 | 0.453 | 0 | 4 | MT1X |
| 46 | 0 | 1.10199825 | 0.972 | 0.413 | 0 | 4 | GATM |
| 47 | 0 | 1.06819814 | 1 | 0.959 | 0 | 4 | FTL |
| 48 | 0 | 0.98756068 | 0.794 | 0.286 | 0 | 4 | PCK1 |
| 49 | 0 | 0.96600914 | 0.98 | 0.54 | 0 | 4 | PEBP1 |
| 50 | 0 | 0.96161909 | 0.798 | 0.302 | 0 | 4 | GSTA1 |
| 51 | 0 | 1.25868522 | 0.938 | 0.484 | 0 | 5 | MT-ND4L |
| 52 | 0 | 1.23694343 | 0.998 | 0.8 | 0 | 5 | MT-ND5 |
| 53 | 0 | 1.19341647 | 1 | 0.979 | 0 | 5 | MT-CO3 |
| 54 | 0 | 1.1649156 | 1 | 0.961 | 0 | 5 | MT-ND3 |
| 55 | 0 | 1.14091094 | 1 | 0.975 | 0 | 5 | MT-ND2 |
| 56 | 0 | 1.13965987 | 1 | 0.946 | 0 | 5 | MT-ND1 |
| 57 | 0 | 1.10044059 | 1 | 0.976 | 0 | 5 | MT-ATP6 |
| 58 | 0 | 1.08164834 | 1 | 0.987 | 0 | 5 | MT-CO1 |
| 59 | 0 | 1.03414262 | 1 | 0.96 | 0 | 5 | MT-CYB |
| 60 | 0 | 1.02467779 | 0.999 | 0.524 | 0 | 5 | ALDOB |
| 61 | 0 | 1.24314122 | 1 | 0.623 | 0 | 6 | GPX3 |
| 62 | 0 | 1.13078167 | 0.821 | 0.14 | 0 | 6 | HMGCS2 |
| 63 | 0 | 1.05595761 | 0.983 | 0.43 | 0 | 6 | GATM |
| 64 | 0 | 1.00986355 | 0.997 | 0.504 | 0 | 6 | MT1G |
| 65 | 0 | 0.99720586 | 0.992 | 0.484 | 0 | 6 | PDZK1IP1 |
| 66 | 0 | 0.9834094 | 0.892 | 0.326 | 0 | 6 | NAT8 |
| 67 | 0 | 0.98214461 | 0.903 | 0.328 | 0 | 6 | MT1H |
| 68 | 0 | 0.97420194 | 0.911 | 0.327 | 0 | 6 | FABP1 |
| 69 | 0 | 0.9460348 | 0.85 | 0.286 | 0 | 6 | ACAA2 |
| 70 | 0 | 0.93525367 | 0.998 | 0.637 | 0 | 6 | APOE |
| 71 | 0 | 2.44704985 | 0.914 | 0.127 | 0 | 7 | GNLY |
| 72 | 0 | 1.80778093 | 0.895 | 0.182 | 0 | 7 | CCL4 |
| 73 | 0 | 1.74949827 | 0.96 | 0.201 | 0 | 7 | NKG7 |
| 74 | 0 | 1.42462495 | 0.914 | 0.048 | 0 | 7 | KLRD1 |
| 75 | 0 | 1.37757054 | 0.792 | 0.062 | 0 | 7 | GZMB |
| 76 | 0 | 1.33480919 | 0.459 | 0.081 | 0 | 7 | CCL3 |
| 77 | 0 | 1.16028312 | 0.878 | 0.232 | 0 | 7 | CCL5 |
| 78 | 0 | 1.15548564 | 0.789 | 0.086 | 0 | 7 | PRF1 |
| 79 | 0 | 1.0114638 | 0.599 | 0.081 | 0 | 7 | KLRB1 |
| 80 | 0 | 0.96600929 | 0.44 | 0.094 | 0 | 7 | AREG |
| 81 | 3.76E−18 | 1.52583829 | 1 | 0.445 | 8.92E−14 | 8 | SPP1 |
| 82 | 7.21E−16 | 1.42089274 | 1 | 0.653 | 1.71E−11 | 8 | GPX3 |
| 83 | 2.27E−14 | 1.18926861 | 0.931 | 0.382 | 5.38E−10 | 8 | UGT2B7 |
| 84 | 1.48E−13 | 0.95470434 | 0.897 | 0.314 | 3.51E−09 | 8 | PRAP1 |
| 85 | 4.07E−13 | 1.13838265 | 1 | 0.981 | 9.65E−09 | 8 | MT-CO3 |
| 86 | 9.31E−13 | 1.05724685 | 1 | 0.988 | 2.21E−08 | 8 | MT-CO1 |
| 87 | 1.73E−12 | 1.07296567 | 1 | 0.964 | 4.10E−08 | 8 | MT-ND3 |
| 88 | 5.68E−12 | 0.97427297 | 1 | 0.978 | 1.35E−07 | 8 | MT-ATP6 |
| 89 | 9.07E−12 | 0.96476107 | 1 | 0.964 | 2.15E−07 | 8 | MT-CYB |
| 90 | 3.61E−11 | 0.98715804 | 0.931 | 0.501 | 8.56E−07 | 8 | FXYD2 |
| 91 | 0 | 2.71352687 | 0.991 | 0.424 | 0 | 9 | HLA-DRA |
| 92 | 0 | 2.5778438 | 0.984 | 0.31 | 0 | 9 | HLA-DPB1 |
| 93 | 0 | 2.46341823 | 0.977 | 0.293 | 0 | 9 | HLA-DPA1 |
| 94 | 0 | 2.39136426 | 0.998 | 0.552 | 0 | 9 | CD74 |
| 95 | 0 | 2.26894004 | 0.976 | 0.364 | 0 | 9 | HLA-DRB1 |
| 96 | 0 | 2.10175443 | 0.805 | 0.051 | 0 | 9 | C1QB |
| 97 | 0 | 2.06032349 | 0.745 | 0.072 | 0 | 9 | APOC1 |
| 98 | 0 | 2.0016182 | 0.825 | 0.06 | 0 | 9 | C1QA |
| 99 | 0 | 1.94458984 | 0.927 | 0.14 | 0 | 9 | HLA-DQA1 |
| 100 | 0 | 1.88145967 | 0.934 | 0.166 | 0 | 9 | HLA-DQB1 |
| 101 | 0 | 1.72824537 | 0.842 | 0.069 | 0 | 10 | NDUFA4L2 |
| 102 | 0 | 1.66167028 | 0.842 | 0.648 | 0 | 10 | MT2A |
| 103 | 0 | 1.6268939 | 0.822 | 0.079 | 0 | 10 | NNMT |
| 104 | 0 | 1.36102074 | 0.656 | 0.1 | 0 | 10 | ANGPTL4 |
| 105 | 0 | 1.21385537 | 0.885 | 0.382 | 0 | 10 | VIM |
| 106 | 0 | 1.16031882 | 0.817 | 0.335 | 0 | 10 | LDHA |
| 107 | 0 | 1.12599097 | 0.725 | 0.171 | 0 | 10 | CD24 |
| 108 | 0 | 1.10758215 | 0.513 | 0.031 | 0 | 10 | HILPDA |
| 109 | 0 | 1.05688842 | 0.808 | 0.426 | 0 | 10 | ENO1 |
| 110 | 6.67E−109 | 0.97975237 | 0.65 | 0.499 | 1.58E−104 | 10 | MT1X |
| 111 | 0 | 2.33244027 | 0.804 | 0.097 | 0 | 11 | S100A9 |
| 112 | 0 | 2.20084206 | 0.862 | 0.125 | 0 | 11 | LYZ |
| 113 | 0 | 2.11811648 | 0.66 | 0.077 | 0 | 11 | S100A8 |
| 114 | 0 | 1.57751049 | 0.846 | 0.149 | 0 | 11 | CTSS |
| 115 | 0 | 1.52897618 | 0.823 | 0.088 | 0 | 11 | LST1 |
| 116 | 0 | 1.52336256 | 0.897 | 0.184 | 0 | 11 | TYROBP |
| 117 | 0 | 1.48021636 | 0.838 | 0.101 | 0 | 11 | AIF1 |
| 118 | 0 | 1.42727896 | 0.855 | 0.142 | 0 | 11 | FCER1G |
| 119 | 0 | 1.41727498 | 0.775 | 0.023 | 0 | 11 | FCN1 |
| 120 | 0 | 1.28698636 | 0.94 | 0.395 | 0 | 11 | SRGN |
| 121 | 0 | 2.26328526 | 0.815 | 0.079 | 0 | 12 | SPARCL1 |
| 122 | 0 | 2.12502012 | 0.673 | 0.063 | 0 | 12 | ENPP2 |
| 123 | 0 | 2.03985709 | 0.76 | 0.041 | 0 | 12 | ESM1 |
| 124 | 0 | 1.97612784 | 0.887 | 0.321 | 0 | 12 | IGFBP7 |
| 125 | 0 | 1.8991249 | 0.661 | 0.074 | 0 | 12 | IGFBP3 |
| 126 | 0 | 1.65551409 | 0.792 | 0.092 | 0 | 12 | SPARC |
| 127 | 0 | 1.45106962 | 0.657 | 0.097 | 0 | 12 | INSR |
| 128 | 0 | 1.36260562 | 0.662 | 0.048 | 0 | 12 | FLT1 |
| 129 | 0 | 1.31506409 | 0.697 | 0.044 | 0 | 12 | PLVAP |
| 130 | 0 | 1.29378217 | 0.744 | 0.136 | 0 | 12 | GSN |
| 131 | 0 | 2.68282907 | 0.957 | 0.112 | 0 | 13 | DEFB1 |
| 132 | 0 | 1.87923948 | 0.402 | 0.033 | 0 | 13 | UMOD |
| 133 | 0 | 1.17112143 | 0.535 | 0.011 | 0 | 13 | KNG1 |
| 134 | 0 | 1.13296445 | 0.367 | 0.007 | 0 | 13 | TMEM52B |
| 135 | 0 | 1.09950055 | 0.848 | 0.181 | 0 | 13 | CD24 |
| 136 | 0 | 1.09567238 | 0.691 | 0.06 | 0 | 13 | CKB |
| 137 | 0 | 0.99196574 | 0.734 | 0.166 | 0 | 13 | MPC1 |
| 138 | 4.51E−306 | 1.4764746 | 0.96 | 0.494 | 1.07E−301 | 13 | FXYD2 |
| 139 | 1.03E−287 | 1.13034106 | 0.935 | 0.508 | 2.45E−283 | 13 | S100A6 |
| 140 | 5.68E−110 | 0.93374621 | 0.565 | 0.266 | 1.35E−105 | 13 | ATP1B1 |
| 141 | 0 | 1.9326565 | 0.869 | 0.087 | 0 | 14 | TIMP3 |
| 142 | 0 | 1.85377099 | 0.711 | 0.054 | 0 | 14 | IGFBP5 |
| 143 | 0 | 1.44099376 | 0.86 | 0.091 | 0 | 14 | IFI27 |
| 144 | 0 | 1.41482812 | 0.913 | 0.222 | 0 | 14 | IFITM3 |
| 145 | 0 | 1.37809838 | 0.85 | 0.069 | 0 | 14 | RNASE1 |
| 146 | 0 | 1.20988824 | 0.694 | 0.044 | 0 | 14 | SDPR |
| 147 | 0 | 1.1527853 | 0.847 | 0.127 | 0 | 14 | GSN |
| 148 | 0 | 1.07172159 | 0.672 | 0.047 | 0 | 14 | SLC9A3R2 |
| 149 | 0 | 1.03895791 | 0.754 | 0.048 | 0 | 14 | RAMP2 |
| 150 | 0 | 1.037622 | 0.578 | 0.074 | 0 | 14 | HES1 |
| 151 | 0 | 1.71459859 | 1 | 0.888 | 0 | 15 | MALAT1 |
| 152 | 0 | 0.82034934 | 0.66 | 0.204 | 0 | 15 | XIST |
| 153 | 0 | 0.7910443 | 0.777 | 0.298 | 0 | 15 | PTPRC |
| 154 | 1.54E−206 | 0.58901669 | 0.558 | 0.2 | 3.66E−202 | 15 | CD2 |
| 155 | 2.77E−203 | 0.616086 | 0.634 | 0.28 | 6.56E−199 | 15 | SRSF7 |
| 156 | 2.69E−169 | 0.5132314 | 0.453 | 0.161 | 6.37E−165 | 15 | RP11-347P5.1 |
| 157 | 4.97E−169 | 0.63232946 | 0.78 | 0.388 | 1.18E−164 | 15 | CXCR4 |
| 158 | 2.09E−151 | 0.53292704 | 0.845 | 0.574 | 4.95E−147 | 15 | DDX5 |
| 159 | 6.52E−100 | 0.56461798 | 0.875 | 0.718 | 1.55E−95 | 15 | HLA-A |
| 160 | 1.81E−85 | 0.5533682 | 0.999 | 0.91 | 4.29E−81 | 15 | B2M |
| 161 | 0 | 1.74160925 | 0.95 | 0.257 | 0 | 16 | HSPA1A |
| 162 | 0 | 1.66441292 | 0.659 | 0.092 | 0 | 16 | HSPA6 |
| 163 | 0 | 1.53033619 | 0.884 | 0.125 | 0 | 16 | HSPA1B |
| 164 | 0 | 1.4500448 | 0.905 | 0.295 | 0 | 16 | DNAJB1 |
| 165 | 0 | 1.41620777 | 0.98 | 0.651 | 0 | 16 | HSP90AA1 |
| 166 | 0 | 0.90530788 | 0.875 | 0.421 | 0 | 16 | JUN |
| 167 | 0 | 0.71224943 | 0.91 | 0.516 | 0 | 16 | HSPA8 |
| 168 | 0 | 0.70123161 | 0.645 | 0.238 | 0 | 16 | DNAJA1 |
| 169 | 0 | 0.67311439 | 0.739 | 0.263 | 0 | 16 | CCL5 |
| 170 | 0 | 0.66830377 | 0.92 | 0.471 | 0 | 16 | HSPE1 |
| 171 | 0 | 1.65442859 | 0.898 | 0.012 | 0 | 17 | TMEM213 |
| 172 | 0 | 1.47064702 | 0.867 | 0.003 | 0 | 17 | ATP6V1G3 |
| 173 | 4.74E−180 | 1.35131245 | 0.734 | 0.08 | 1.12E−175 | 17 | CA12 |
| 174 | 8.97E−160 | 1.25824941 | 0.719 | 0.078 | 2.13E−155 | 17 | IGFBP5 |
| 175 | 1.96E−137 | 1.74014461 | 0.445 | 0.037 | 4.65E−133 | 17 | KRT7 |
| 176 | 2.03E−131 | 1.64517193 | 0.797 | 0.124 | 4.80E−127 | 17 | DEFB1 |
| 177 | 5.39E−120 | 1.43682033 | 0.836 | 0.156 | 1.28E−115 | 17 | CD9 |
| 178 | 1.87E−109 | 1.20360434 | 0.875 | 0.205 | 4.42E−105 | 17 | ATP6AP2 |
| 179 | 1.06E−84 | 1.44831262 | 0.539 | 0.086 | 2.51E−80 | 17 | WFDC2 |
| 180 | 9.74E−69 | 1.32910867 | 0.281 | 0.028 | 2.31E−64 | 17 | SPINK1 |
| 181 | 1.53E−213 | 1.24160693 | 0.571 | 0.16 | 3.63E−209 | 18 | CLU |
| 182 | 1.32E−187 | 0.97132163 | 0.389 | 0.083 | 3.14E−183 | 18 | WFDC2 |
| 183 | 1.04E−125 | 0.98571432 | 1 | 0.988 | 2.46E−121 | 18 | MT-CO1 |
| 184 | 2.98E−122 | 1.01049368 | 0.998 | 0.981 | 7.07E−118 | 18 | MT-CO3 |
| 185 | 2.88E−120 | 0.94648935 | 0.997 | 0.963 | 6.83E−116 | 18 | MT-CYB |
| 186 | 4.38E−118 | 1.10049066 | 0.992 | 0.964 | 1.04E−113 | 18 | MT-ND3 |
| 187 | 7.41E−110 | 0.9345293 | 0.997 | 0.98 | 1.76E−105 | 18 | MT-CO2 |
| 188 | 1.05E−104 | 0.90605286 | 0.967 | 0.814 | 2.49E−100 | 18 | MT-ND5 |
| 189 | 1.29E−103 | 0.92084702 | 0.995 | 0.95 | 3.06E−99 | 18 | MT-ND1 |
| 190 | 1.76E−95 | 0.94143902 | 0.997 | 0.977 | 4.18E−91 | 18 | MT-ND2 |
| 191 | 0 | 3.15465769 | 0.867 | 0.036 | 0 | 19 | SAA1 |
| 192 | 0 | 2.11233827 | 0.658 | 0.019 | 0 | 19 | SAA2 |
| 193 | 0 | 1.76645735 | 0.923 | 0.096 | 0 | 19 | NNMT |
| 194 | 0 | 1.70513005 | 0.832 | 0.157 | 0 | 19 | CLU |
| 195 | 0 | 1.54113296 | 0.826 | 0.089 | 0 | 19 | NDUFA4L2 |
| 196 | 0 | 1.35434435 | 0.556 | 0.011 | 0 | 19 | REG1A |
| 197 | 0 | 1.29822347 | 0.793 | 0.041 | 0 | 19 | KRT19 |
| 198 | 0 | 1.25184393 | 0.808 | 0.192 | 0 | 19 | SOD2 |
| 199 | 0 | 1.23087313 | 0.637 | 0.063 | 0 | 19 | TGFBI |
| 200 | 2.25E−237 | 1.27198774 | 0.836 | 0.347 | 5.33E−233 | 19 | LDHA |
| 201 | 0 | 2.93105821 | 0.829 | 0.036 | 0 | 20 | RGS5 |
| 202 | 0 | 2.47695582 | 0.882 | 0.041 | 0 | 20 | ACTA2 |
| 203 | 0 | 2.39597548 | 0.971 | 0.33 | 0 | 20 | IGFBP7 |
| 204 | 0 | 2.1496319 | 0.804 | 0.019 | 0 | 20 | TAGLN |
| 205 | 0 | 1.92891179 | 0.893 | 0.162 | 0 | 20 | CALD1 |
| 206 | 0 | 1.67413539 | 0.841 | 0.1 | 0 | 20 | MGP |
| 207 | 0 | 1.6505872 | 0.798 | 0.043 | 0 | 20 | MYL9 |
| 208 | 0 | 1.46988035 | 0.765 | 0.015 | 0 | 20 | TPM2 |
| 209 | 0 | 1.43556621 | 0.72 | 0.018 | 0 | 20 | BGN |
| 210 | 2.48E−186 | 1.58169189 | 0.817 | 0.39 | 5.88E−182 | 20 | ADIRF |
| 211 | 0 | 3.04365017 | 1 | 0.066 | 0 | 21 | IGFBP5 |
| 212 | 0 | 2.45433947 | 0.992 | 0.05 | 0 | 21 | EMCN |
| 213 | 0 | 2.3759083 | 0.985 | 0.04 | 0 | 21 | PLAT |
| 214 | 0 | 2.19181514 | 0.957 | 0.022 | 0 | 21 | CRHBP |
| 215 | 0 | 2.17178005 | 0.961 | 0.097 | 0 | 21 | MGP |
| 216 | 0 | 1.76014631 | 0.949 | 0.109 | 0 | 21 | IFI27 |
| 217 | 0 | 1.74477144 | 0.946 | 0.067 | 0 | 21 | PLPP3 |
| 218 | 0 | 1.64998693 | 0.98 | 0.105 | 0 | 21 | TIMP3 |
| 219 | 0 | 1.61516995 | 0.967 | 0.238 | 0 | 21 | IFITM3 |
| 220 | 0 | 1.59153063 | 0.959 | 0.102 | 0 | 21 | TGFBR2 |
| 221 | 0 | 5.18224192 | 1 | 0.263 | 0 | 22 | HBA2 |
| 222 | 0 | 4.69608842 | 1 | 0.094 | 0 | 22 | HBA1 |
| 223 | 0 | 0.50837446 | 0.39 | 0.001 | 0 | 22 | HBD |
| 224 | 1.04E−264 | 4.99810634 | 1 | 0.394 | 2.47E−260 | 22 | HBB |
| 225 | 6.37E−129 | 0.50514144 | 0.469 | 0.089 | 1.51E−124 | 22 | SLC25A37 |
| 226 | 1.43E−18 | 0.37196617 | 0.399 | 0.206 | 3.39E−14 | 22 | IGKC |
| 227 | 1.25E−11 | 0.26342162 | 0.286 | 0.155 | 2.96E−07 | 22 | LYZ |
| 228 | 0 | 2.13634022 | 0.979 | 0.044 | 0 | 23 | KRT19 |
| 229 | 0 | 2.12355431 | 0.89 | 0.021 | 0 | 23 | SPINK1 |
| 230 | 0 | 2.11726456 | 0.932 | 0.015 | 0 | 23 | S100P |
| 231 | 0 | 1.92242401 | 0.879 | 0.004 | 0 | 23 | KRT13 |
| 232 | 0 | 1.81045045 | 0.803 | 0.025 | 0 | 23 | S100A2 |
| 233 | 0 | 1.66936232 | 0.814 | 0.004 | 0 | 23 | KRT17 |
| 234 | 0 | 1.66397077 | 0.913 | 0.033 | 0 | 23 | ELF3 |
| 235 | 0 | 1.48724278 | 0.84 | 0.062 | 0 | 23 | AQP3 |
| 236 | 0 | 1.45798791 | 0.84 | 0.084 | 0 | 23 | ID1 |
| 237 | 3.45E−200 | 1.65673793 | 0.99 | 0.511 | 8.17E−196 | 23 | S100A6 |
| 238 | 0 | 2.83901268 | 1 | 0.344 | 0 | 24 | MTRNR2L8 |
| 239 | 0 | 2.61433644 | 1 | 0.299 | 0 | 24 | MTRNR2L12 |
| 240 | 1.53E−182 | 1.2415044 | 1 | 0.981 | 3.61E−178 | 24 | MT-ND4 |
| 241 | 5.06E−182 | 1.2942469 | 1 | 0.95 | 1.20E−177 | 24 | MT-ND1 |
| 242 | 6.69E−181 | 1.17523073 | 1 | 0.98 | 1.59E−176 | 24 | MT-CO2 |
| 243 | 1.20E−173 | 1.23963965 | 1 | 0.981 | 2.85E−169 | 24 | MT-CO3 |
| 244 | 1.04E−169 | 1.19669558 | 1 | 0.977 | 2.47E−165 | 24 | MT-ATP6 |
| 245 | 2.15E−168 | 1.12527158 | 1 | 0.964 | 5.10E−164 | 24 | MT-CYB |
| 246 | 8.37E−168 | 1.14858834 | 1 | 0.988 | 1.98E−163 | 24 | MT-CO1 |
| 247 | 1.68E−148 | 1.10043084 | 1 | 0.964 | 3.98E−144 | 24 | MT-ND3 |
| 248 | 0 | 2.85605374 | 0.462 | 0.016 | 0 | 25 | JCHAIN |
| 249 | 0 | 1.66449328 | 0.456 | 0.005 | 0 | 25 | IGHM |
| 250 | 0 | 0.99575881 | 0.68 | 0.005 | 0 | 25 | CD79A |
| 251 | 6.12E−243 | 3.53659658 | 0.45 | 0.027 | 1.45E−238 | 25 | IGHG1 |
| 252 | 6.70E−177 | 2.73910845 | 0.284 | 0.015 | 1.59E−172 | 25 | IGHG4 |
| 253 | 1.05E−154 | 3.47126356 | 0.331 | 0.023 | 2.49E−150 | 25 | IGHG3 |
| 254 | 1.28E−103 | 4.81169846 | 0.775 | 0.205 | 3.02E−99 | 25 | IGKC |
| 255 | 1.24E−86 | 3.71764698 | 0.29 | 0.031 | 2.94E−82 | 25 | IGLC3 |
| 256 | 8.45E−66 | 4.06843384 | 0.426 | 0.082 | 2.00E−61 | 25 | IGLC2 |
| 257 | 1.89E−36 | 3.81621056 | 0.385 | 0.103 | 4.48E−32 | 25 | IGHA1 |
| 258 | 0 | 4.32178839 | 1 | 0.022 | 0 | 26 | TPSB2 |
| 259 | 0 | 4.08934084 | 1 | 0.018 | 0 | 26 | TPSAB1 |
| 260 | 0 | 1.83084649 | 0.92 | 0.002 | 0 | 26 | CPA3 |
| 261 | 0 | 1.3536883 | 0.389 | 0.001 | 0 | 26 | CTSG |
| 262 | 0 | 1.2822239 | 0.814 | 0.002 | 0 | 26 | MS4A2 |
| 263 | 0 | 1.23803482 | 0.805 | 0.003 | 0 | 26 | HPGDS |
| 264 | 1.84E−131 | 1.4677551 | 0.832 | 0.117 | 4.36E−127 | 26 | AREG |
| 265 | 1.26E−86 | 1.78849966 | 0.92 | 0.25 | 2.98E−82 | 26 | CD69 |
| 266 | 3.95E−53 | 1.26733156 | 0.938 | 0.434 | 9.35E−49 | 26 | JUN |
| 267 | 7.27E−48 | 1.27935318 | 0.903 | 0.418 | 1.72E−43 | 26 | FOS |
| 268 | 1.62E−69 | 1.29130392 | 0.684 | 0.028 | 3.84E−65 | 27 | TAGLN |
| 269 | 8.08E−65 | 1.14802376 | 0.842 | 0.046 | 1.92E−60 | 27 | RGS5 |
| 270 | 8.40E−59 | 1.31744483 | 0.842 | 0.051 | 1.99E−54 | 27 | ACTA2 |
| 271 | 2.33E−46 | 1.9511384 | 1 | 0.1 | 5.53E−42 | 27 | HBA1 |
| 272 | 8.07E−26 | 1.47564845 | 0.947 | 0.17 | 1.91E−21 | 27 | CALD1 |
| 273 | 8.63E−22 | 2.04749914 | 1 | 0.268 | 2.05E−17 | 27 | HBA2 |
| 274 | 2.11E−17 | 1.84492504 | 1 | 0.338 | 5.00E−13 | 27 | IGFBP7 |
| 275 | 3.11E−17 | 2.33249827 | 1 | 0.398 | 7.36E−13 | 27 | HBB |
| 276 | 5.56E−17 | 1.58003263 | 0.947 | 0.297 | 1.32E−12 | 27 | LGALS1 |
| 277 | 2.68E−16 | 1.24370923 | 0.789 | 0.175 | 6.36E−12 | 27 | TIMP1 |

TABLE 2
Differential expression of genes between ccRCC tumor cluster
and normal epithelium cluster, Related to FIG. 1.

| Symbol | p_val | avg_logFC | pct.1 | pct.2 | p_val_adj | log10p-value | #NAME? |
|---|---|---|---|---|---|---|---|
| NDUFA4L2 | 1.00E−16 | 1.592 | 0.764 | 0.107 | 0 | −16.000 | 16.000 |
| NNMT | 0 | 1.519 | 0.747 | 0.091 | 0 | −16.000 | 16.000 |
| ANGPTL4 | 0 | 1.332 | 0.581 | 0.032 | 0 | −16.000 | 16.000 |
| CD24 | 0 | 1.223 | 0.645 | 0.045 | 0 | −16.000 | 16.000 |
| RNASE1 | 0 | −0.870 | 0.011 | 0.506 | 0 | −16.000 | 16.000 |
| TMSB4X | 0 | −0.956 | 0.957 | 0.998 | 0 | −16.000 | 16.000 |
| PECAM1 | 0 | −0.968 | 0.018 | 0.526 | 0 | −16.000 | 16.000 |
| IFI27 | 0 | −1.067 | 0.072 | 0.61 | 0 | −16.000 | 16.000 |
| B2M | 0 | −1.127 | 0.992 | 1 | 0 | −16.000 | 16.000 |
| PLVAP | 0 | −1.179 | 0.014 | 0.554 | 0 | −16.000 | 16.000 |
| FLT1 | 0 | −1.201 | 0.012 | 0.508 | 0 | −16.000 | 16.000 |
| MGP | 0 | −1.225 | 0.032 | 0.548 | 0 | −16.000 | 16.000 |
| GSN | 0 | −1.287 | 0.044 | 0.606 | 0 | −16.000 | 16.000 |
| SPARC | 0 | −1.524 | 0.077 | 0.681 | 0 | −16.000 | 16.000 |
| ESM1 | 0 | −1.898 | 0.028 | 0.665 | 0 | −16.000 | 16.000 |
| ENPP2 | 0 | −1.911 | 0.033 | 0.57 | 0 | −16.000 | 16.000 |
| IGFBP7 | 0 | −2.009 | 0.301 | 0.82 | 0 | −16.000 | 16.000 |
| SPARCL1 | 0 | −2.161 | 0.035 | 0.716 | 0 | −16.000 | 16.000 |
| TIMP3 | 1.57E−305 | −1.112 | 0.029 | 0.53 | 3.71E−301 | −304.805 | 304.805 |
| GAPDH | 5.36E−305 | 0.903 | 0.991 | 0.906 | 1.27E−300 | −304.271 | 304.271 |
| RPL41 | 1.81E−302 | 0.813 | 0.997 | 0.98 | 4.29E−298 | −301.742 | 301.742 |
| A2M | 5.03E−301 | −1.129 | 0.022 | 0.512 | 1.19E−296 | −300.299 | 300.299 |
| PLPP1 | 9.49E−297 | −0.921 | 0.026 | 0.515 | 2.25E−292 | −296.023 | 296.023 |
| SLC9A3R2 | 8.58E−294 | −0.848 | 0.007 | 0.479 | 2.03E−289 | −293.067 | 293.067 |
| RPLP0 | 3.35E−292 | 0.911 | 0.959 | 0.767 | 7.93E−288 | −291.475 | 291.475 |
| RAMP2 | 1.07E−291 | −0.772 | 0.01 | 0.48 | 2.54E−287 | −290.971 | 290.971 |
| CD59 | 2.36E−287 | −1.041 | 0.141 | 0.637 | 5.59E−283 | −286.627 | 286.627 |
| EPAS1 | 4.91E−286 | −0.914 | 0.04 | 0.521 | 1.16E−281 | −285.308 | 285.308 |
| LDHA | 9.30E−286 | 1.078 | 0.738 | 0.268 | 2.20E−281 | −285.032 | 285.032 |
| RPL10 | 2.14E−284 | 0.759 | 0.999 | 0.98 | 5.07E−280 | −283.670 | 283.670 |
| ENO1 | 1.72E−283 | 1.086 | 0.746 | 0.284 | 4.07E−279 | −282.765 | 282.765 |
| ADGRL4 | 2.01E−282 | −0.763 | 0.003 | 0.458 | 4.77E−278 | −281.696 | 281.696 |
| FTH1 | 7.83E−281 | 0.810 | 0.997 | 0.971 | 1.86E−276 | −280.106 | 280.106 |
| RPS2 | 8.37E−277 | 0.819 | 0.99 | 0.925 | 1.98E−272 | −276.077 | 276.077 |
| MT2A | 1.21E−274 | 1.712 | 0.877 | 0.581 | 2.87E−270 | −273.917 | 273.917 |
| PRSS23 | 1.88E−273 | −0.938 | 0.028 | 0.487 | 4.46E−269 | −272.725 | 272.725 |
| RGCC | 6.63E−270 | −1.053 | 0.051 | 0.519 | 1.57E−265 | −269.179 | 269.179 |
| GNG11 | 3.93E−268 | −0.908 | 0.125 | 0.602 | 9.31E−264 | −267.406 | 267.406 |
| CLEC14A | 5.29E−260 | −0.742 | 0.005 | 0.431 | 1.25E−255 | −259.277 | 259.277 |
| HLA-B | 3.02E−259 | −0.937 | 0.805 | 0.968 | 7.15E−255 | −258.521 | 258.521 |
| RPS8 | 2.57E−255 | 0.820 | 0.985 | 0.919 | 6.10E−251 | −254.590 | 254.590 |
| HLA-C | 9.30E−255 | −0.883 | 0.711 | 0.948 | 2.20E−250 | −254.032 | 254.032 |
| VWF | 3.95E−254 | −0.941 | 0.015 | 0.441 | 9.37E−250 | −253.403 | 253.403 |
| RPL13 | 1.07E−247 | 0.679 | 0.999 | 0.975 | 2.53E−243 | −246.972 | 246.972 |
| RPS19 | 2.92E−247 | 0.724 | 0.995 | 0.963 | 6.93E−243 | −246.534 | 246.534 |
| SPRY1 | 1.05E−245 | −0.783 | 0.022 | 0.443 | 2.49E−241 | −244.979 | 244.979 |
| HLA-E | 5.14E−242 | −0.977 | 0.272 | 0.689 | 1.22E−237 | −241.289 | 241.289 |
| NOTCH4 | 1.84E−241 | −0.707 | 0.006 | 0.408 | 4.37E−237 | −240.734 | 240.734 |
| RPL36 | 2.36E−241 | 0.785 | 0.982 | 0.909 | 5.59E−237 | −240.627 | 240.627 |
| RPS18 | 7.49E−239 | 0.682 | 0.997 | 0.965 | 1.78E−234 | −238.125 | 238.125 |
| SDPR | 5.84E−235 | −0.782 | 0.037 | 0.454 | 1.39E−230 | −234.233 | 234.233 |
| RPS5 | 2.02E−234 | 0.768 | 0.949 | 0.792 | 4.79E−230 | −233.694 | 233.694 |
| RPL18A | 4.16E−230 | 0.726 | 0.985 | 0.932 | 9.87E−226 | −229.380 | 229.380 |
| RBP7 | 1.60E−228 | −0.900 | 0.014 | 0.406 | 3.80E−224 | −227.796 | 227.796 |
| ID3 | 3.71E−225 | −0.681 | 0.028 | 0.428 | 8.78E−221 | −224.431 | 224.431 |
| HSPG2 | 5.37E−225 | −0.705 | 0.009 | 0.39 | 1.27E−220 | −224.270 | 224.270 |
| HILPDA | 2.49E−224 | 1.014 | 0.448 | 0.02 | 5.91E−220 | −223.604 | 223.604 |
| EDNRB | 2.82E−221 | −0.666 | 0.007 | 0.383 | 6.69E−217 | −220.549 | 220.549 |
| ITGB1 | 3.80E−221 | −0.874 | 0.13 | 0.55 | 9.00E−217 | −220.421 | 220.421 |
| CRYAB | 7.28E−218 | 0.921 | 0.752 | 0.389 | 1.73E−213 | −217.138 | 217.138 |
| ENG | 1.83E−217 | −0.602 | 0.008 | 0.38 | 4.34E−213 | −216.737 | 216.737 |
| ESAM | 3.66E−216 | −0.521 | 0.006 | 0.372 | 8.67E−212 | −215.437 | 215.437 |
| MT1X | 3.76E−215 | 1.637 | 0.673 | 0.287 | 8.91E−211 | −214.425 | 214.425 |
| RPL39 | 2.78E−212 | 0.665 | 0.987 | 0.939 | 6.58E−208 | −211.557 | 211.557 |
| TCF4 | 1.52E−210 | −0.592 | 0.008 | 0.37 | 3.60E−206 | −209.819 | 209.819 |
| EGFL7 | 1.52E−207 | −0.531 | 0.005 | 0.36 | 3.60E−203 | −206.819 | 206.819 |
| HYAL2 | 4.07E−204 | −0.524 | 0.006 | 0.356 | 9.64E−200 | −203.391 | 203.391 |
| RPS21 | 2.93E−201 | 0.641 | 0.947 | 0.797 | 6.95E−197 | −200.533 | 200.533 |
| SEC14L1 | 4.88E−200 | −0.534 | 0.026 | 0.39 | 1.16E−195 | −199.312 | 199.312 |
| MIF | 2.94E−198 | 0.812 | 0.691 | 0.316 | 6.96E−194 | −197.532 | 197.532 |
| TMEM204 | 1.50E−194 | −0.473 | 0.004 | 0.337 | 3.56E−190 | −193.823 | 193.823 |
| RPS27 | 5.12E−194 | 0.590 | 0.997 | 0.987 | 1.21E−189 | −193.291 | 193.291 |
| RAMP3 | 1.27E−192 | −0.530 | 0.007 | 0.342 | 3.02E−188 | −191.895 | 191.895 |
| RPL21 | 1.92E−192 | 0.614 | 0.993 | 0.958 | 4.54E−188 | −191.717 | 191.717 |
| FKBP1A | 4.00E−190 | −0.738 | 0.164 | 0.561 | 9.47E−186 | −189.398 | 189.398 |
| RARRES2 | 1.28E−189 | 0.595 | 0.411 | 0.027 | 3.03E−185 | −188.894 | 188.894 |
| RPS4X | 1.58E−188 | 0.606 | 0.99 | 0.946 | 3.76E−184 | −187.800 | 187.800 |
| YWHAH | 1.98E−188 | −0.648 | 0.058 | 0.429 | 4.68E−184 | −187.704 | 187.704 |
| RNASET2 | 9.60E−186 | 0.689 | 0.451 | 0.061 | 2.28E−181 | −185.018 | 185.018 |
| RPL36A | 9.94E−185 | 0.661 | 0.89 | 0.69 | 2.36E−180 | −184.003 | 184.003 |
| RPL3 | 1.22E−184 | 0.553 | 0.992 | 0.952 | 2.89E−180 | −183.914 | 183.914 |
| RPL26 | 3.30E−183 | 0.611 | 0.987 | 0.94 | 7.83E−179 | −182.481 | 182.481 |
| CD74 | 1.28E−182 | −0.771 | 0.611 | 0.883 | 3.05E−178 | −181.891 | 181.891 |
| APP | 2.71E−182 | −0.785 | 0.152 | 0.528 | 6.43E−178 | −181.567 | 181.567 |
| LDB2 | 3.89E−182 | −0.447 | 0.003 | 0.316 | 9.21E−178 | −181.410 | 181.410 |
| INSR | 4.53E−180 | −1.118 | 0.144 | 0.511 | 1.07E−175 | −179.344 | 179.344 |
| PCAT19 | 4.96E−180 | −0.439 | 0.002 | 0.312 | 1.18E−175 | −179.304 | 179.304 |
| ZFAS1 | 6.84E−179 | 0.721 | 0.52 | 0.126 | 1.62E−174 | −178.165 | 178.165 |
| RPL31 | 5.12E−178 | 0.612 | 0.979 | 0.911 | 1.21E−173 | −177.291 | 177.291 |
| RPL37 | 7.58E−178 | 0.597 | 0.981 | 0.9 | 1.80E−173 | −177.120 | 177.120 |
| SPTBN1 | 3.68E−176 | −0.525 | 0.04 | 0.383 | 8.71E−172 | −175.435 | 175.435 |
| BCAM | 5.52E−176 | −0.505 | 0.029 | 0.363 | 1.31E−171 | −175.258 | 175.258 |
| APOLD1 | 2.90E−175 | −0.459 | 0.008 | 0.317 | 6.87E−171 | −174.538 | 174.538 |
| ADGRF5 | 3.39E−175 | −0.437 | 0.002 | 0.304 | 8.03E−171 | −174.470 | 174.470 |
| ANGPT2 | 1.50E−173 | −0.597 | 0.011 | 0.32 | 3.56E−169 | −172.823 | 172.823 |
| RPS28 | 6.90E−172 | 0.516 | 0.994 | 0.974 | 1.63E−167 | −171.161 | 171.161 |
| PDGFD | 3.13E−171 | −0.399 | 0.001 | 0.295 | 7.41E−167 | −170.505 | 170.505 |
| MEF2C | 8.69E−171 | −0.414 | 0.003 | 0.3 | 2.06E−166 | −170.061 | 170.061 |
| BNIP3 | 1.00E−170 | 0.594 | 0.41 | 0.042 | 2.38E−166 | −169.998 | 169.998 |
| SEPW1 | 2.38E−170 | −0.691 | 0.151 | 0.516 | 5.65E−166 | −169.623 | 169.623 |
| EGLN3 | 9.32E−170 | 0.514 | 0.347 | 0.009 | 2.21E−165 | −169.030 | 169.030 |
| RPL13A | 7.92E−169 | 0.499 | 0.997 | 0.974 | 1.88E−164 | −168.101 | 168.101 |
| RPS14 | 2.35E−168 | 0.538 | 0.988 | 0.959 | 5.58E−164 | −167.628 | 167.628 |
| BTNL9 | 5.99E−168 | −0.502 | 0.003 | 0.297 | 1.42E−163 | −167.223 | 167.223 |
| IFITM3 | 1.89E−166 | −0.756 | 0.478 | 0.771 | 4.49E−162 | −165.723 | 165.723 |
| GIMAP7 | 5.05E−166 | −0.458 | 0.013 | 0.315 | 1.20E−161 | −165.297 | 165.297 |
| INHBB | 8.18E−166 | −0.473 | 0.003 | 0.294 | 1.94E−161 | −165.087 | 165.087 |
| IFITM2 | 1.05E−163 | −0.639 | 0.177 | 0.541 | 2.50E−159 | −162.977 | 162.977 |
| COL4A1 | 2.25E−163 | −0.650 | 0.017 | 0.319 | 5.34E−159 | −162.647 | 162.647 |
| ITGA6 | 1.10E−162 | −0.457 | 0.011 | 0.307 | 2.61E−158 | −161.959 | 161.959 |
| RPL23A | 1.14E−162 | 0.559 | 0.983 | 0.946 | 2.69E−158 | −161.945 | 161.945 |
| RPS15 | 2.54E−162 | 0.529 | 0.988 | 0.95 | 6.03E−158 | −161.595 | 161.595 |
| RPL11 | 4.21E−162 | 0.518 | 0.994 | 0.957 | 9.98E−158 | −161.376 | 161.376 |
| RPL6 | 4.95E−162 | 0.569 | 0.965 | 0.85 | 1.17E−157 | −161.305 | 161.305 |
| RPL7 | 8.06E−161 | 0.536 | 0.987 | 0.931 | 1.91E−156 | −160.094 | 160.094 |
| RPS6 | 2.82E−160 | 0.541 | 0.99 | 0.964 | 6.69E−156 | −159.549 | 159.549 |
| RPL10A | 6.45E−160 | 0.546 | 0.947 | 0.8 | 1.53E−155 | −159.191 | 159.191 |
| ECSCR.1 | 1.42E−159 | −0.375 | 0.007 | 0.294 | 3.35E−155 | −158.849 | 158.849 |
| TGFBR2 | 6.28E−159 | −0.438 | 0.016 | 0.313 | 1.49E−154 | −158.202 | 158.202 |
| LINC01320 | 2.49E−158 | 0.538 | 0.333 | 0.011 | 5.90E−154 | −157.604 | 157.604 |
| MGST1 | 1.94E−157 | 0.513 | 0.363 | 0.026 | 4.61E−153 | −156.711 | 156.711 |
| PTPRB | 2.02E−157 | −0.393 | 0.003 | 0.28 | 4.78E−153 | −156.695 | 156.695 |
| TMEM88 | 2.39E−157 | −0.403 | 0.003 | 0.281 | 5.66E−153 | −156.622 | 156.622 |
| ARHGAP29 | 2.79E−157 | −0.472 | 0.048 | 0.369 | 6.61E−153 | −156.555 | 156.555 |
| EFNB2 | 2.36E−156 | −0.456 | 0.003 | 0.277 | 5.58E−152 | −155.628 | 155.628 |
| RPL24 | 3.23E−156 | 0.572 | 0.948 | 0.825 | 7.66E−152 | −155.490 | 155.490 |
| RPS16 | 1.03E−155 | 0.561 | 0.97 | 0.885 | 2.45E−151 | −154.986 | 154.986 |
| RPL18 | 1.15E−153 | 0.574 | 0.953 | 0.835 | 2.74E−149 | −152.937 | 152.937 |
| TACC1 | 1.66E−153 | −0.497 | 0.055 | 0.375 | 3.94E−149 | −152.779 | 152.779 |
| EMCN | 6.59E−153 | −0.471 | 0.003 | 0.272 | 1.56E−148 | −152.181 | 152.181 |
| PLIN2 | 1.15E−152 | 0.738 | 0.501 | 0.141 | 2.73E−148 | −151.939 | 151.939 |
| TM4SF1 | 4.31E−152 | −0.531 | 0.039 | 0.346 | 1.02E−147 | −151.365 | 151.365 |
| EEF1A1 | 5.77E−151 | 0.509 | 0.998 | 0.984 | 1.37E−146 | −150.239 | 150.239 |
| PLPP3 | 9.34E−150 | −0.554 | 0.037 | 0.336 | 2.21E−145 | −149.030 | 149.030 |
| CALCRL | 1.63E−149 | −0.385 | 0.007 | 0.278 | 3.86E−145 | −148.788 | 148.788 |
| FAM84A | 4.88E−149 | −0.425 | 0.011 | 0.286 | 1.16E−144 | −148.311 | 148.311 |
| RPS25 | 5.24E−149 | 0.529 | 0.985 | 0.934 | 1.24E−144 | −148.281 | 148.281 |
| COL4A2 | 2.85E−148 | −0.490 | 0.016 | 0.295 | 6.75E−144 | −147.546 | 147.546 |
| GJA1 | 6.42E−148 | −0.385 | 0.003 | 0.265 | 1.52E−143 | −147.193 | 147.193 |
| RPL35 | 6.51E−148 | 0.530 | 0.976 | 0.9 | 1.54E−143 | −147.186 | 147.186 |
| AQP1 | 1.03E−146 | −0.532 | 0.018 | 0.298 | 2.44E−142 | −145.988 | 145.988 |
| PODXL | 1.71E−146 | −0.404 | 0.003 | 0.263 | 4.06E−142 | −145.766 | 145.766 |
| TPI1 | 4.15E−146 | 0.616 | 0.752 | 0.501 | 9.85E−142 | −145.381 | 145.381 |
| RPS20 | 1.05E−144 | 0.522 | 0.975 | 0.903 | 2.48E−140 | −143.980 | 143.980 |
| GIMAP4 | 1.07E−144 | −0.394 | 0.023 | 0.306 | 2.54E−140 | −143.971 | 143.971 |
| RPL38 | 3.76E−144 | 0.534 | 0.952 | 0.843 | 8.92E−140 | −143.425 | 143.425 |
| CLDN5 | 2.46E−143 | −0.587 | 0.006 | 0.266 | 5.84E−139 | −142.608 | 142.608 |
| KRT18 | 1.35E−141 | 0.504 | 0.34 | 0.03 | 3.21E−137 | −140.869 | 140.869 |
| SERPINA1 | 5.51E−141 | 0.758 | 0.37 | 0.05 | 1.31E−136 | −140.259 | 140.259 |
| RPS15A | 5.97E−141 | 0.483 | 0.986 | 0.944 | 1.41E−136 | −140.224 | 140.224 |
| RPL34 | 1.66E−139 | 0.491 | 0.99 | 0.979 | 3.95E−135 | −138.779 | 138.779 |
| NUPR1 | 2.44E−138 | 0.501 | 0.366 | 0.048 | 5.77E−134 | −137.613 | 137.613 |
| ALDOA | 3.00E−138 | 0.662 | 0.693 | 0.433 | 7.11E−134 | −137.523 | 137.523 |
| RPL35A | 3.00E−138 | 0.497 | 0.981 | 0.929 | 7.12E−134 | −137.523 | 137.523 |
| CD34 | 6.92E−138 | −0.333 | 0.005 | 0.254 | 1.64E−133 | −137.160 | 137.160 |
| RPS17 | 2.23E−137 | 0.493 | 0.976 | 0.908 | 5.29E−133 | −136.652 | 136.652 |
| MGST2 | 1.57E−136 | −0.389 | 0.038 | 0.323 | 3.72E−132 | −135.804 | 135.804 |
| ITGA1 | 4.92E−136 | −0.411 | 0.029 | 0.303 | 1.17E−131 | −135.308 | 135.308 |
| NPM1 | 5.51E−136 | 0.656 | 0.607 | 0.287 | 1.31E−131 | −135.259 | 135.259 |
| RPL19 | 7.19E−136 | 0.461 | 0.986 | 0.933 | 1.70E−131 | −135.143 | 135.143 |
| VEGFA | 2.97E−135 | 0.503 | 0.291 | 0.01 | 7.04E−131 | −134.527 | 134.527 |
| LIFR | 6.25E−135 | −0.395 | 0.022 | 0.288 | 1.48E−130 | −134.204 | 134.204 |
| TMEM176B | 7.81E−135 | 0.453 | 0.325 | 0.026 | 1.85E−130 | −134.107 | 134.107 |
| TMEM176A | 1.33E−134 | 0.463 | 0.327 | 0.028 | 3.15E−130 | −133.877 | 133.877 |
| SOD2 | 5.46E−134 | 0.742 | 0.45 | 0.121 | 1.29E−129 | −133.263 | 133.263 |
| MYL12A | 1.21E−132 | −0.579 | 0.191 | 0.531 | 2.88E−128 | −131.916 | 131.916 |
| CLU | 1.89E−132 | 0.596 | 0.408 | 0.084 | 4.47E−128 | −131.724 | 131.724 |
| PKM | 1.14E−131 | 0.594 | 0.474 | 0.143 | 2.71E−127 | −130.942 | 130.942 |
| TSC22D1 | 3.04E−130 | −0.666 | 0.209 | 0.532 | 7.22E−126 | −129.516 | 129.516 |
| RPL5 | 1.33E−129 | 0.487 | 0.943 | 0.822 | 3.14E−125 | −128.878 | 128.878 |
| CA9 | 5.71E−129 | 0.393 | 0.265 | 0.003 | 1.35E−124 | −128.244 | 128.244 |
| CRIP2 | 1.03E−128 | −0.469 | 0.103 | 0.409 | 2.44E−124 | −127.987 | 127.987 |
| HLA-A | 1.50E−128 | −0.554 | 0.807 | 0.948 | 3.55E−124 | −127.824 | 127.824 |
| VAMP5 | 4.49E−127 | −0.533 | 0.128 | 0.436 | 1.07E−122 | −126.347 | 126.347 |
| RPS7 | 1.55E−126 | 0.453 | 0.97 | 0.879 | 3.68E−122 | −125.808 | 125.808 |
| GNAS | 3.80E−126 | −0.404 | 0.048 | 0.325 | 9.00E−122 | −125.421 | 125.421 |
| RPS27A | 5.89E−125 | 0.428 | 0.991 | 0.964 | 1.40E−120 | −124.230 | 124.230 |
| IVNS1ABP | 4.69E−124 | −0.487 | 0.056 | 0.332 | 1.11E−119 | −123.329 | 123.329 |
| ATP1B1 | 1.61E−122 | 0.514 | 0.344 | 0.052 | 3.82E−118 | −121.793 | 121.793 |
| KRT8 | 1.99E−122 | 0.417 | 0.306 | 0.028 | 4.72E−118 | −121.701 | 121.701 |
| CCDC85B | 4.24E−121 | −0.388 | 0.053 | 0.325 | 1.01E−116 | −120.373 | 120.373 |
| RPL9 | 1.34E−117 | 0.472 | 0.98 | 0.93 | 3.18E−113 | −116.872 | 116.872 |
| MYL12B | 4.69E−117 | −0.507 | 0.361 | 0.674 | 1.11E−112 | −116.328 | 116.328 |
| RPS3A | 5.77E−114 | 0.437 | 0.986 | 0.94 | 1.37E−109 | −113.239 | 113.239 |
| NOL3 | 2.42E−113 | 0.372 | 0.286 | 0.027 | 5.75E−109 | −112.615 | 112.615 |
| FAU | 2.02E−112 | 0.424 | 0.954 | 0.87 | 4.79E−108 | −111.694 | 111.694 |
| RPL12 | 4.80E−112 | 0.453 | 0.977 | 0.908 | 1.14E−107 | −111.319 | 111.319 |
| SNHG8 | 6.15E−111 | 0.458 | 0.402 | 0.104 | 1.46E−106 | −110.211 | 110.211 |
| RPL28 | 3.29E−110 | 0.426 | 0.989 | 0.941 | 7.79E−106 | −109.483 | 109.483 |
| RPS13 | 1.31E−109 | 0.433 | 0.969 | 0.89 | 3.09E−105 | −108.884 | 108.884 |
| RPL8 | 1.03E−108 | 0.417 | 0.973 | 0.9 | 2.45E−104 | −107.986 | 107.986 |
| RPLP2 | 4.71E−108 | 0.392 | 0.992 | 0.961 | 1.12E−103 | −107.327 | 107.327 |
| RPS3 | 6.90E−108 | 0.429 | 0.976 | 0.922 | 1.63E−103 | −107.161 | 107.161 |
| RPS24 | 9.12E−108 | 0.446 | 0.98 | 0.935 | 2.16E−103 | −107.040 | 107.040 |
| PGK1 | 2.97E−107 | 0.503 | 0.47 | 0.173 | 7.05E−103 | −106.527 | 106.527 |
| RPS23 | 1.07E−105 | 0.396 | 0.982 | 0.941 | 2.53E−101 | −104.972 | 104.972 |
| RPL27A | 2.58E−105 | 0.387 | 0.984 | 0.949 | 6.11E−101 | −104.589 | 104.589 |
| NACA | 1.94E−103 | 0.403 | 0.914 | 0.794 | 4.60E−99 | −102.712 | 102.712 |
| CD9 | 3.59E−103 | −0.514 | 0.149 | 0.429 | 8.52E−99 | −102.445 | 102.445 |
| NPDC1 | 1.58E−101 | −0.299 | 0.036 | 0.265 | 3.73E−97 | −100.803 | 100.803 |
| RPS12 | 1.83E−101 | 0.387 | 0.987 | 0.951 | 4.34E−97 | −100.737 | 100.737 |
| SRP14 | 1.32E−100 | −0.490 | 0.658 | 0.844 | 3.14E−96 | −99.878 | 99.878 |
| PTRF | 2.83E−99 | −0.367 | 0.081 | 0.333 | 6.71E−95 | −98.548 | 98.548 |
| RPL30 | 8.41E−99 | 0.396 | 0.969 | 0.892 | 1.99E−94 | −98.075 | 98.075 |
| RPS11 | 1.97E−96 | 0.424 | 0.926 | 0.845 | 4.66E−92 | −95.706 | 95.706 |
| RPL22 | 3.35E−96 | 0.390 | 0.92 | 0.813 | 7.94E−92 | −95.475 | 95.475 |
| RPL23 | 3.63E−96 | 0.396 | 0.896 | 0.765 | 8.60E−92 | −95.440 | 95.440 |
| CALM1 | 7.21E−94 | −0.445 | 0.288 | 0.582 | 1.71E−89 | −93.142 | 93.142 |
| STC1 | 1.52E−93 | −0.628 | 0.123 | 0.375 | 3.60E−89 | −92.819 | 92.819 |
| RPLP1 | 7.87E−93 | 0.351 | 0.996 | 0.977 | 1.87E−88 | −92.104 | 92.104 |
| ANXA4 | 1.22E−91 | 0.383 | 0.271 | 0.041 | 2.89E−87 | −90.914 | 90.914 |
| ITM2B | 2.10E−91 | −0.422 | 0.89 | 0.969 | 4.98E−87 | −90.677 | 90.677 |
| STOM | 2.39E−91 | −0.338 | 0.07 | 0.304 | 5.67E−87 | −90.621 | 90.621 |
| SOX4 | 1.79E−90 | 0.455 | 0.27 | 0.042 | 4.23E−86 | −89.748 | 89.748 |
| TPT1 | 5.80E−90 | 0.361 | 0.99 | 0.95 | 1.38E−85 | −89.236 | 89.236 |
| RHOA | 3.91E−89 | −0.365 | 0.111 | 0.365 | 9.26E−85 | −88.408 | 88.408 |
| HNRNPA1 | 5.01E−89 | 0.444 | 0.747 | 0.588 | 1.19E−84 | −88.300 | 88.300 |
| PTMA | 1.12E−88 | −0.459 | 0.941 | 0.967 | 2.65E−84 | −87.952 | 87.952 |
| EEF2 | 2.22E−86 | 0.444 | 0.686 | 0.492 | 5.26E−82 | −85.654 | 85.654 |
| RPL7A | 6.20E−86 | 0.376 | 0.937 | 0.836 | 1.47E−81 | −85.208 | 85.208 |
| HES1 | 5.54E−84 | −0.552 | 0.11 | 0.346 | 1.31E−79 | −83.256 | 83.256 |
| RHOC | 6.77E−84 | −0.381 | 0.14 | 0.394 | 1.61E−79 | −83.169 | 83.169 |
| ARHGDIB | 2.23E−83 | −0.325 | 0.104 | 0.353 | 5.28E−79 | −82.652 | 82.652 |
| OAZ2 | 9.57E−82 | −0.339 | 0.078 | 0.3 | 2.27E−77 | −81.019 | 81.019 |
| HTRA1 | 1.10E−81 | −0.292 | 0.053 | 0.26 | 2.60E−77 | −80.960 | 80.960 |
| COX7C | 8.74E−81 | 0.312 | 0.925 | 0.78 | 2.07E−76 | −80.059 | 80.059 |
| EFNA1 | 1.47E−79 | −0.283 | 0.073 | 0.292 | 3.49E−75 | −78.832 | 78.832 |
| HLA-DRB1 | 3.87E−78 | −0.485 | 0.246 | 0.502 | 9.18E−74 | −77.412 | 77.412 |
| DUSP1 | 8.64E−78 | 0.606 | 0.715 | 0.548 | 2.05E−73 | −77.063 | 77.063 |
| NFIB | 1.47E−75 | −0.328 | 0.107 | 0.333 | 3.49E−71 | −74.832 | 74.832 |
| ARGLU1 | 1.44E−73 | −0.306 | 0.08 | 0.291 | 3.41E−69 | −72.842 | 72.842 |
| RPS9 | 4.00E−73 | 0.326 | 0.956 | 0.876 | 9.47E−69 | −72.398 | 72.398 |
| CFL1 | 4.81E−73 | −0.361 | 0.379 | 0.643 | 1.14E−68 | −72.318 | 72.318 |
| RPL4 | 3.59E−70 | 0.342 | 0.791 | 0.654 | 8.52E−66 | −69.445 | 69.445 |
| EIF4EBP1 | 3.03E−69 | 0.287 | 0.256 | 0.058 | 7.17E−65 | −68.519 | 68.519 |
| EGR1 | 9.85E−69 | 0.414 | 0.263 | 0.064 | 2.33E−64 | −68.007 | 68.007 |
| SPP1 | 2.15E−68 | 0.544 | 0.693 | 0.534 | 5.09E−64 | −67.668 | 67.668 |
| TMEM50A | 3.91E−68 | −0.260 | 0.079 | 0.279 | 9.27E−64 | −67.408 | 67.408 |
| DARS | 3.93E−68 | 0.276 | 0.278 | 0.073 | 9.31E−64 | −67.406 | 67.406 |
| GMFG | 1.69E−67 | −0.273 | 0.071 | 0.268 | 4.02E−63 | −66.771 | 66.771 |
| RPS29 | 1.88E−67 | 0.310 | 0.977 | 0.928 | 4.46E−63 | −66.726 | 66.726 |
| PDK4 | 4.91E−67 | 0.582 | 0.455 | 0.241 | 1.16E−62 | −66.309 | 66.309 |
| SLC16A3 | 5.63E−67 | 0.271 | 0.252 | 0.058 | 1.33E−62 | −66.250 | 66.250 |
| ADIRF | 1.22E−66 | 0.468 | 0.468 | 0.227 | 2.89E−62 | −65.914 | 65.914 |
| FTL | 7.85E−66 | 0.281 | 0.988 | 0.95 | 1.86E−61 | −65.105 | 65.105 |
| RPS26 | 1.53E−65 | 0.292 | 0.933 | 0.862 | 3.64E−61 | −64.814 | 64.814 |
| MT1E | 1.42E−64 | 0.704 | 0.289 | 0.086 | 3.38E−60 | −63.846 | 63.846 |
| SRGN | 1.59E−64 | −0.479 | 0.216 | 0.448 | 3.78E−60 | −63.797 | 63.797 |
| PFDN5 | 1.19E−63 | 0.334 | 0.801 | 0.71 | 2.83E−59 | −62.923 | 62.923 |
| CD99 | 1.64E−63 | −0.315 | 0.138 | 0.349 | 3.90E−59 | −62.784 | 62.784 |
| LGALS3 | 6.30E−63 | 0.316 | 0.295 | 0.092 | 1.49E−58 | −62.200 | 62.200 |
| CYB5A | 8.43E−63 | 0.351 | 0.745 | 0.586 | 2.00E−58 | −62.074 | 62.074 |
| HLA-DRA | 1.74E−62 | −0.415 | 0.352 | 0.579 | 4.12E−58 | −61.760 | 61.760 |
| APOC1 | 2.80E−62 | 0.392 | 0.305 | 0.102 | 6.63E−58 | −61.553 | 61.553 |
| HLA-DRB5 | 5.44E−62 | −0.347 | 0.156 | 0.375 | 1.29E−57 | −61.264 | 61.264 |
| MT-CYB | 1.74E−61 | −0.356 | 0.924 | 0.979 | 4.11E−57 | −60.761 | 60.761 |
| UBC | 1.83E−61 | 0.415 | 0.916 | 0.873 | 4.35E−57 | −60.737 | 60.737 |
| BNIP3L | 2.72E−61 | 0.280 | 0.282 | 0.086 | 6.44E−57 | −60.566 | 60.566 |
| MYL6 | 5.12E−61 | −0.344 | 0.723 | 0.869 | 1.21E−56 | −60.291 | 60.291 |
| APLP2 | 1.87E−60 | −0.341 | 0.156 | 0.361 | 4.42E−56 | −59.729 | 59.729 |
| SLC25A6 | 6.61E−60 | 0.335 | 0.619 | 0.424 | 1.57E−55 | −59.180 | 59.180 |
| CCND1 | 7.78E−60 | 0.342 | 0.271 | 0.08 | 1.84E−55 | −59.109 | 59.109 |
| PNRC1 | 1.06E−58 | 0.377 | 0.412 | 0.204 | 2.50E−54 | −57.976 | 57.976 |
| PPDPF | 1.58E−57 | 0.405 | 0.586 | 0.408 | 3.74E−53 | −56.802 | 56.802 |
| CNN3 | 1.90E−57 | −0.317 | 0.162 | 0.365 | 4.50E−53 | −56.722 | 56.722 |
| GNB2L1 | 2.24E−57 | 0.305 | 0.886 | 0.79 | 5.31E−53 | −56.650 | 56.650 |
| YWHAB | 2.82E−57 | −0.263 | 0.104 | 0.297 | 6.69E−53 | −56.549 | 56.549 |
| TMA7 | 9.93E−56 | −0.291 | 0.261 | 0.497 | 2.35E−51 | −55.003 | 55.003 |
| BTF3 | 1.55E−55 | 0.344 | 0.65 | 0.494 | 3.68E−51 | −54.810 | 54.810 |
| GABARAPL2 | 2.32E−55 | −0.252 | 0.089 | 0.27 | 5.50E−51 | −54.634 | 54.634 |
| HMGB1 | 4.32E−55 | −0.369 | 0.391 | 0.589 | 1.02E−50 | −54.364 | 54.364 |
| SEC61G | 5.53E−55 | 0.347 | 0.417 | 0.214 | 1.31E−50 | −54.257 | 54.257 |
| HERPUD1 | 5.23E−54 | −0.343 | 0.165 | 0.365 | 1.24E−49 | −53.281 | 53.281 |
| DDX5 | 8.44E−54 | −0.256 | 0.51 | 0.731 | 2.00E−49 | −53.073 | 53.073 |
| CAPZA2 | 2.41E−53 | −0.293 | 0.147 | 0.339 | 5.71E−49 | −52.618 | 52.618 |
| S100A10 | 2.51E−53 | 0.396 | 0.641 | 0.459 | 5.95E−49 | −52.600 | 52.600 |
| RPL27 | 4.71E−53 | 0.260 | 0.92 | 0.835 | 1.12E−48 | −52.327 | 52.327 |
| NAP1L1 | 5.41E−53 | 0.324 | 0.407 | 0.202 | 1.28E−48 | −52.267 | 52.267 |
| RPS10 | 1.84E−52 | 0.312 | 0.756 | 0.644 | 4.37E−48 | −51.734 | 51.734 |
| ATP5G2 | 6.31E−52 | 0.324 | 0.616 | 0.45 | 1.49E−47 | −51.200 | 51.200 |
| RPSA | 1.70E−51 | 0.298 | 0.766 | 0.637 | 4.03E−47 | −50.770 | 50.770 |
| RPL14 | 6.53E−51 | 0.272 | 0.94 | 0.862 | 1.55E−46 | −50.185 | 50.185 |
| IGFBP3 | 1.25E−50 | −0.837 | 0.45 | 0.573 | 2.96E−46 | −49.903 | 49.903 |
| HLA-DPA1 | 3.43E−50 | −0.253 | 0.164 | 0.371 | 8.14E−46 | −49.464 | 49.464 |
| EIF3E | 8.91E−50 | 0.322 | 0.402 | 0.206 | 2.11E−45 | −49.050 | 49.050 |
| NDRG1 | 2.49E−49 | 0.265 | 0.272 | 0.099 | 5.89E−45 | −48.604 | 48.604 |
| KTN1 | 5.35E−49 | −0.262 | 0.145 | 0.331 | 1.27E−44 | −48.272 | 48.272 |
| UBA52 | 1.94E−47 | 0.257 | 0.923 | 0.868 | 4.59E−43 | −46.713 | 46.713 |
| NBEAL1 | 1.07E−46 | 0.317 | 0.407 | 0.211 | 2.55E−42 | −45.969 | 45.969 |
| GLTSCR2 | 3.66E−46 | 0.291 | 0.421 | 0.229 | 8.67E−42 | −45.437 | 45.437 |
| IGFBP4 | 4.53E−46 | −0.262 | 0.161 | 0.345 | 1.07E−41 | −45.344 | 45.344 |
| SEC62 | 2.16E−45 | −0.335 | 0.271 | 0.452 | 5.11E−41 | −44.666 | 44.666 |
| ATP5E | 1.08E−43 | −0.285 | 0.762 | 0.886 | 2.56E−39 | −42.967 | 42.967 |
| POMP | 2.30E−43 | −0.318 | 0.238 | 0.419 | 5.46E−39 | −42.638 | 42.638 |
| RPL17 | 1.04E−42 | 0.316 | 0.517 | 0.35 | 2.48E−38 | −41.981 | 41.981 |
| IL32 | 2.96E−42 | 0.308 | 0.394 | 0.202 | 7.01E−38 | −41.529 | 41.529 |
| ST13 | 3.44E−42 | 0.262 | 0.33 | 0.153 | 8.15E−38 | −41.464 | 41.464 |
| FOS | 3.49E−42 | 0.520 | 0.508 | 0.351 | 8.28E−38 | −41.457 | 41.457 |
| PEBP1 | 1.22E−41 | 0.273 | 0.703 | 0.565 | 2.90E−37 | −40.912 | 40.912 |
| TMSB10 | 3.82E−41 | 0.313 | 0.99 | 0.98 | 9.06E−37 | −40.418 | 40.418 |
| H3F3A | 2.58E−37 | −0.250 | 0.611 | 0.771 | 6.11E−33 | −36.588 | 36.588 |
| FXYD2 | 6.38E−36 | 0.318 | 0.653 | 0.533 | 1.51E−31 | −35.195 | 35.195 |
| BST2 | 2.18E−35 | −0.266 | 0.227 | 0.386 | 5.17E−31 | −34.661 | 34.661 |
| RAC1 | 5.11E−34 | −0.274 | 0.294 | 0.453 | 1.21E−29 | −33.291 | 33.291 |
| HSP90B1 | 1.35E−33 | −0.257 | 0.232 | 0.391 | 3.20E−29 | −32.870 | 32.870 |
| GSTP1 | 1.13E−30 | 0.296 | 0.464 | 0.319 | 2.67E−26 | −29.948 | 29.948 |
| NEAT1 | 2.77E−27 | −0.618 | 0.558 | 0.607 | 6.57E−23 | −26.557 | 26.557 |
| VIM | 7.41E−26 | 0.324 | 0.804 | 0.709 | 1.76E−21 | −25.130 | 25.130 |
| MTRNR2L8 | 1.03E−23 | −0.311 | 0.219 | 0.349 | 2.44E−19 | −22.987 | 22.987 |
| ZFP36 | 1.01E−19 | 0.271 | 0.446 | 0.338 | 2.38E−15 | −18.997 | 18.997 |
| S100A6 | 2.99E−18 | −0.274 | 0.551 | 0.667 | 7.10E−14 | −17.524 | 17.524 |
| KLF6 | 1.19E−17 | 0.265 | 0.414 | 0.312 | 2.81E−13 | −16.925 | 16.925 |
| JUN | 1.10E−16 | 0.291 | 0.436 | 0.335 | 2.60E−12 | −15.960 | 15.960 |
| DDIT4 | 7.83E−13 | 0.268 | 0.539 | 0.464 | 1.86E−08 | −12.106 | 12.106 |
| MALAT1 | 4.36E−11 | −0.679 | 0.987 | 0.995 | 1.03E−06 | −10.361 | 10.361 |
| MT1G | 3.45E−06 | −0.252 | 0.445 | 0.496 | 0.081821324 | −5.462 | 5.462 |
| GPX3 | 2.88E−05 | −0.299 | 0.741 | 0.699 | 0.682517049 | −4.541 | 4.541 |

TABLE 3
List of oligonucleotides used for qPCR.

| Gene | Forward primer | | Reverse primer | |
|---|---|---|---|---|
| ACAD9 | TCGGAGATGGGTTTAAGGTG | SEQ ID NO: | CGTAAGCCTTCTGAGCCATC | SEQ ID NO: |
| | | 45 | | 61 |
| ACOT7 | CCGAAAACATCCTCACAGGT | SEQ ID NO: | GTTCCTCCACTTGGTCTCCA | SEQ ID NO: |
| | | 46 | | 62 |
| APOE | GGTCGCTTTTGGGATTACCT | SEQ ID NO: | TCCAGTTCCGATTTGTAGGC | SEQ ID NO: |
| | | 47 | | 63 |
| β-Actin | CATGTACGTTGCTATCCAGG | SEQ ID NO: | CTCCTTAATGTCACGCACGA | SEQ ID NO: |
| | | 48 | T | 64 |
| CPTIA | GAAGATGGCAGAAGCTCACC | SEQ ID NO: | TGGCGTACATCGTTGTCAT | SEQ ID NO: |
| | | 49 | | 65 |
| DBI | TGGCCACTACAAACAAGCAA | SEQ ID NO: | TGGCACAGTAACCAAATCCA | SEQ ID NO: |
| | | 50 | | 66 |
| EPAS1 | GACATGAAGTTCACCTACTGTGAT | SEQ ID NO: | CGGAGTCTAGCGCATGGTA | SEQ ID NO: |
| | G | 51 | | 67 |
| FABP7 | CCAGCTGGGAGAAGAGTTTG | SEQ ID NO: | CTCATAGTGGCGAACAGCAA | SEQ ID NO: |
| | | 52 | | 68 |
| HIF1a | CAGCTATTTGCGTGTGAGGA | SEQ ID NO: | TTCATCTGTGCTTTCATGTCA | SEQ ID NO: |
| | | 53 | TC | 69 |
| IGFBP3 | CAGAGACTCGAGCACAGCAC | SEQ ID NO: | GATGACCGGGGTTTAAAGGT | SEQ ID NO: |
| | | 54 | | 70 |
| LOX | GGACTGAGAAAGGGGAAAGG | SEQ ID NO: | GGACGTGGCTCACAGAAAAT | SEQ ID NO: |
| | | 55 | | 71 |
| PLIN4 | CAGATGCAGGAAGCATCAAA | SEQ ID NO: | GCGACTAAAAGGCACTCTGG | SEQ ID NO: |
| | | 56 | | 72 |
| RARRES2 | GTGCAAAGTCAGGCCCAATG | SEQ ID NO: | TTGGGTCTCTATGGGGCAGT | SEQ ID NO: |
| | | 57 | | 73 |
| RORC | CAGTGAGAGCCCAGAAGGAC | SEQ ID NO: | TCATCCCATCCATTTTTGGT | SEQ ID NO: |
| | | 58 | | 74 |
| SMPD3 | GAGGGCTGCATCTCTACCAG | SEQ ID NO: | ACCCTGTGAAGTGAGGGTTG | SEQ ID NO: |
| | | 59 | | 75 |
| VEGFA | TCCTCACACCATTGAAACCA | SEQ ID NO: | GATCCTGCCCTGTCTCTCTG | SEQ ID NO: |
| | | 60 | | 76 |

TABLE 4
List of recombinant DNAs.

| | Name in | | |
|---|---|---|---|
| TRCN Name | Manuscript | Gene Name | |
| TRCN0000063443 | shRARRES2-1 | RARRES2; | SEQ ID |
| | | CCAATGGGAGG | NO: 77 |
| | | AAACGGAAAT | |
| TRCN0000373361 | shRARRES2-2 | RARRES2; | SEQ ID |
| | | CCCATAGAGAC | NO: 78 |
| | | CCAAGTTCTG | |
| TRCN0000373359 | shRARRES2-3 | RARRES2; | SEQ ID |
| | | CAGGTGGCCCT | NO: 79 |
| | | GGAGGAATTT | |
| TRCN0000003803 | shHIF2α | EPAS1 | |
| siGENOME | | | |
| SMARTpool | | Target | |
| siRNA | Gene Name | Sequence | |
| M-021441-01 | KLF6 | GCCUAGAGCU | SEQ ID |
| | | GGAACGUUA | NO: 41 |
| M-021441-02 | KLF6 | GCAGGAAAGU | SEQ ID |
| | | UUACACCAA | NO: 42 |
| M-021441-03 | KLF6 | UGCAAGAAGU | SEQ ID |
| | | GAUGAGUUA | NO: 43 |
| M-021441-04 | KLF6 | AAAUUGAGCU | SEQ ID |
| | | CCUCUGUCA | NO: 44 |

TABLE 5
List of noncoding guide RNAs for CRISPR-Cas9.

| Gene number | Sequence | |
|---|---|---|
| RARRES2 A1 | 5′-CACCGGACCAGTGTGGAGAGCGCCG-3′ | SEQ ID NO: 17 |
| RARRES2 A2 | 5′-AAACCGGCGCTCTCCACACTGGTCC-3′ | SEQ ID NO: 18 |
| RARRES2 B1 | 5′-CACCGGCGACGGCTGCTGATCCCTC-3′ | SEQ ID NO: 19 |
| RARRES2 B2 | 5′-AAACGAGGGATCAGCAGCCGTCGCC-3′ | SEQ ID NO: 20 |
| RARRES2 C1 | 5′-CACCGCTATGGGGCAGTGGACCAAC-3′ | SEQ ID NO: 21 |
| RARRES2 C2 | 5′-AAACGTTGGTCCACTGCCCCATAGC-3′ | SEQ ID NO: 22 |
| RARRES2 D1 | 5′-CACCGCCAGTGCTGGCTTAGCTGCG-3′ | SEQ ID NO: 23 |
| RARRES2 D2 | 5′-AAACCGCAGCTAAGCCAGCACTGGC-3′ | SEQ ID NO: 24 |
| RARRES2 E1 | 5′-CACCGCCCTTCTTACCCGCAGAACT-3′ | SEQ ID NO: 25 |
| RARRES2 E2 | 5′-AAACAGTTCTGCGGGTAAGAAGGGC-3′ | SEQ ID NO: 26 |
| RARRES2 F1 | 5′-CACCGATTGGGCCTGACTTTGCACT-3′ | SEQ ID NO: 27 |
| RARRES2 F2 | 5′-AAACAGTGCAAAGTCAGGCCCAATC-3′ | SEQ ID NO: 28 |
| CMKLR1 F1 | 5′-CACCGGAACCACCGCAGCGTTCGCC-3′ | SEQ ID NO: 29 |
| CMKLR1 R1 | 5′-AAACGGCGAACGCTGCGGTGGTTCC-3′ | SEQ ID NO: 30 |
| CMKLR1 F2 | 5′-CACCGCAAACTGCAGCGCAACCGCC-3′ | SEQ ID NO: 31 |
| CMKLR1 R2 | 5′-AAACGGCGGTTGCGCTGCAGTTTGC-3′ | SEQ ID NO: 32 |
| CMKLR1 F3 | 5′-CACCGTGTGGGGTATAGCCGGCACA-3′ | SEQ ID NO: 33 |
| CMKLR1 R3 | 5′-AAACTGTGCCGGCTATACCCCACAC-3′ | SEQ ID NO: 34 |
| CMKLR1 F4 | 5′-CACCGCCATATCACCTATGCCGCCA-3′ | SEQ ID NO: 35 |
| CMKLR1 R4 | 5′-AAACTGGCGGCATAGGTGATATGGC-3′ | SEQ ID NO: 36 |
| CMKLR1 F5 | 5′-CACCGGTATTCATCACCGTAACTGA-3′ | SEQ ID NO: 37 |
| CMKLR1 R5 | 5′-AAACTCAGTTACGGTGATGAATACC-3′ | SEQ ID NO: 38 |
| CMKLR1 F6 | 5′-CACCGGCGCTGCAGTTTGCACACGA-3′ | SEQ ID NO: 39 |
| CMKLR1 R6 | 5′-AAACTCGTGTGCAAACTGCAGCGCC-3′ | SEQ ID NO: 40 |

TABLE 6
List of antibodies.

| Antibodies | Source | Catalog Number |
|---|---|---|
| Rabbit anti-RARRES2 (IB) (Polyclonal) | Proteintech | Cat# 10216-1-AP |
| Rabbit anti-EPAS | Novus | Cat# NB100-122 |
| Mouse ant-β-Actin (Monoclonal) | Sigma-Aldrich | Cat# A1987 |
| Mouse anti-NDUFA9 (Monoclonal) | Abcam | Cat# ab14713 |
| Mouse anti-UQCRC2 (Monoclonal) | Abcam | Cat# ab14745 |
| Mouse anti-COX1 (Monoclonal) | Abcam | Cat# ab14705 |
| Rabbit anti-SDHA (Polyclonal) | Proteintech | Cat# 14865-1-AP |
| Mouse anti-ATP5A (Monoclonal) | Abcam | Cat# ab14748 |
| Rabbit anti-VHL (Polyclonal) | Cell signaling | Cat# 2738S |
| Rabbit anti-p-Akt Ser473 (Polyclonal) | Cell signaling | Cat# 9271S |
| Rabbit anti-p-Akt Thr308 (Polyclonal) | Cell signaling | Cat# 9275S |
| Rabbit anti-Akt (Polyclonal) | Cell signaling | Cat# 9272S |
| Rabbit anti-p-p44/42 MAPK Thr202/Tyr204 (Polyclonal) | Cell signaling | Cat# 9101S |
| Rabbit anti-p44/42 MAPK (Polyclonal) | Cell signaling | Cat# 9102S |
| Mouse anti-KLF6, clone 12A.8.3 (Monoclonal) | Millipore-Sigma | Cat# MABN119 |
| Mouse anti-CMKLR1 (ChemR23, C-7) (Monoclonal) | Santa Cruz | Cat# sc-374570 |
| Rabbit anti-RARRES2 (Polyclonal) (IHC) | Phoenix Pharmaceuticals | Cat# H-002-52 |
| Mouse Gamma Globulin | Jackson Immuno Research | Cat# 015-000-002 |

SEQUENCES
Heavy chain: DNA sequence (396 bp)
SEQ ID NO: 1
ATGGAATGGAACTGGGTCGTTCTCTTCCTCCTGTCATTAA
CTGCAGGTGTCTATTCCCAGGGTCAGATGCAGCAGTCTGG
AGCTGAGCTGGTGAAGCCTGGGGCTTCAGTGAAGCTGTCC
TGCAAGACTTCTGGCTTCACCTTCAGCAGTAGCTATATAA
GTTGGTTGAAGCAGAAGCCTCGACAGAGTCTTGAGTGGAT
TGCATGGATTTATGCTGGAACTGGTGGTACTAGCTATAAT
CAGAAGTTCACAGGCAAGGCCCAACTGACTGTAGACACAT
CCTCCAGCACAGCCTACATGCAACTCAGCAGCCTGACATC
TGAAGACTCTGCCATCTATTACTGTGCAAGCTTCTGGGAC
TCCTGGGGCCAAGGCACCACTCTCACAGTCTCCTCA,
Light chain: DNA sequence (396 bp)
SEQ ID NO: 2
Signal sequence-FR1-CDR1-FR2-CDR2-FR3-CDR3-FR4
ATGAGGTGCCTAGCTGAGTTCCTGGGGCTGCTTGTGCTCT
GGATCCCTGGAGCCATTGGGGATATTGTGATGACTCAGGC
TGCACCCTCTGTACCTGTCACTCCTGGAGAGTCAGTATCC
ATCTCCTGCAGGTCTAGTAAGAGTCTCCTGCATAGTAATG
GCAACACTTACTTGTATTGGTTCCTGCAGAGGCCAGGCCA
GTCTCCTCAGCTCCTGATATATCGGATGTCCAACCTTGCC
TCAGGAGTCCCAGACAGGTTCAGTGGCAGTGGGTCAGGAA
CTGCTTTCACACTGAGAATCAGTAGAGTGGAGGCTGAGGA
TGTGGGTGTTTATTACTGTATGCAACATCTAGAATATCCG
CTCACGTTCGGTGCTGGGACCAAGCTGGAGCTGAAA,
Heavy chain: Amino acid sequence (132 aa)
Signal peptide-FR1-CDR1-FR2-CDR2-FR3-CDR3-FR4
SEQ ID NO: 3
MEWNWVVLFLLSLTAGVYSQGQMQQSGAELVKPGASVKLS
CKTSGFTFSSSYISWLKQKPRQSLEWIAWIYAGTGGTSYN
QKFTGKAQLTVDTSSSTAYMQLSSLTSEDSAIYYCASFWD
SWGQGTTLTVSS,
Light chain: Amino acid sequence (132 aa)
Signal peptide-FR1-CDR1-FR2-CDR2-FR3-CDR3-FR4
SEQ ID NO: 4
MRCLAEFLGLLVLWIPGAIGDIVMTQAAPSVPVTPGESVS
ISCRSSKSLLHSNGNTYLYWFLQRPGQSPQLLIYRMSNLA
SGVPDRFSGSGSGTAFTLRISRVEAEDVGVYYCMQHLEYP
LTFGAGTKLELK,
CDRH1, DNA sequence
SEQ ID NO: 5
AGTAGCTATATAAGT,
CDRH2, DNA sequence
SEQ ID NO: 6
TGGATTTATGCTGGAACTGGTGGTACTAGCTATAATCAGA
AGTTCACAGGC,
CDRH3, DNA sequence
SEQ ID NO: 7
TTCTGGGACTCC,
CDRH1, amino acid sequence
SEQ ID NO: 8
SSYIS,
CDRH2, amino acid sequence
SEQ ID NO: 9
WIYAGTGGTSYNQKFTG,
CDRH3, amino acid sequence
SEQ ID NO: 10
FWDS,
CDRL1, DNA sequence
SEQ ID NO: 11
AGGTCTAGTAAGAGTCTCCTGCATAGTAATGGCAACACTT
ACTTGTAT,
CDRL2, DNA sequence
SEQ ID NO: 12
CGGATGTCCAACCTTGCCTCA,
CDRL3, DNA sequence
SEQ ID NO: 13
ATGCAACATCTAGAATATCCGCTCACG,
CDRL1, amino acid sequence
SEQ ID NO: 14
RSSKSLLHSNGNTYLY
CDRL2, amino acid sequence
SEQ ID NO: 15
RMSNLAS,
CDRL3, amino acid sequence
SEQ ID NO: 16
MQHLEYPLT,
SEQ ID NO: 17
5′-CACCGGACCAGTGTGGAGAGCGCCG-3′
SEQ ID NO: 18
5′-AAACCGGCGCTCTCCACACTGGTCC-3′
SEQ ID NO: 19
5′-CACCGGCGACGGCTGCTGATCCCTC-3′
SEQ ID NO: 20
5′-AAACGAGGGATCAGCAGCCGTCGCC-3′
SEQ ID NO: 21
5′-CACCGCTATGGGGCAGTGGACCAAC-3′
SEQ ID NO: 22
5′-AAACGTTGGTCCACTGCCCCATAGC-3′
SEQ ID NO: 23
5′-CACCGCCAGTGCTGGCTTAGCTGCG-3′
SEQ ID NO: 24
5′- AAACCGCAGCTAAGCCAGCACTGGC-3′
SEQ ID NO: 25
5′-CACCGCCCTTCTTACCCGCAGAACT-3′
SEQ ID NO: 26
5′-AAACAGTTCTGCGGGTAAGAAGGGC-3′
SEQ ID NO: 27
5′- CACCGATTGGGCCTGACTTTGCACT-3′
SEQ ID NO: 28
5′- AAACAGTGCAAAGTCAGGCCCAATC-3′
SEQ ID NO: 29
5′-CACCGGAACCACCGCAGCGTTCGCC-3′
SEQ ID NO: 30
5′-AAACGGCGAACGCTGCGGTGGTTCC-3′
SEQ ID NO: 31
5′-CACCGCAAACTGCAGCGCAACCGCC-3′
SEQ ID NO: 32
5′-AAACGGCGGTTGCGCTGCAGTTTGC-3′
SEQ ID NO: 33
5′-CACCGTGTGGGGTATAGCCGGCACA-3′
SEQ ID NO: 34
5′-AAACTGTGCCGGCTATACCCCACAC-3′
SEQ ID NO: 35
5′-CACCGCCATATCACCTATGCCGCCA-3′
SEQ ID NO: 36
5′-AAACTGGCGGCATAGGTGATATGGC-3′
SEQ ID NO: 37
5′-CACCGGTATTCATCACCGTAACTGA-3′
SEQ ID NO: 38
5′-AAACTCAGTTACGGTGATGAATACC-3′
SEQ ID NO: 39
5′-CACCGGCGCTGCAGTTTGCACACGA-3′
SEQ ID NO: 40
5′-AAACTCGTGTGCAAACTGCAGCGCC-3′
SEQ ID NO: 41
GCCUAGAGCUGGAACGUUA
SEQ ID NO: 42
GCAGGAAAGUUUACACCAA
SEQ ID NO: 43
UGCAAGAAGUGAUGAGUUA
SEQ ID NO: 44
AAAUUGAGCUCCUCUGUCA
SEQ ID NO: 45
TCGGAGATGGGTTTAAGGTG
SEQ ID NO: 46
CCGAAAACATCCTCACAGGT
SEQ ID NO: 47
GGTCGCTTTTGGGATTACCT
SEQ ID NO: 48
CATGTACGTTGCTATCCAGG
SEQ ID NO: 49
GAAGATGGCAGAAGCTCACC
SEQ ID NO: 50
TGGCCACTACAAACAAGCAA
SEQ ID NO: 51
GACATGAAGTTCACCTACTGTGATG
SEQ ID NO: 52
CCAGCTGGGAGAAGAGTTTG
SEQ ID NO: 53
CAGCTATTTGCGTGTGAGGA
SEQ ID NO: 54
CAGAGACTCGAGCACAGCAC
SEQ ID NO: 55
GGACTGAGAAAGGGGAAAGG
SEQ ID NO: 56
CAGATGCAGGAAGCATCAAA
SEQ ID NO: 57
GTGCAAAGTCAGGCCCAATG
SEQ ID NO: 58
CAGTGAGAGCCCAGAAGGAC
SEQ ID NO: 59
GAGGGCTGCATCTCTACCAG
SEQ ID NO: 60
TCCTCACACCATTGAAACCA
SEQ ID NO: 61
CGTAAGCCTTCTGAGCCATC
SEQ ID NO: 62
GTTCCTCCACTTGGTCTCCA
SEQ ID NO: 63
TCCAGTTCCGATTTGTAGGC
SEQ ID NO: 64
CTCCTTAATGTCACGCACGAT
SEQ ID NO: 65
TGGCGTACATCGTTGTCAT
SEQ ID NO: 66
TGGCACAGTAACCAAATCCA
SEQ ID NO: 67
CGGAGTCTAGCGCATGGTA
SEQ ID NO: 68
CTCATAGTGGCGAACAGCAA
SEQ ID NO: 69
TTCATCTGTGCTTTCATGTCATC
SEQ ID NO: 70
GATGACCGGGGTTTAAAGGT
SEQ ID NO: 71
GGACGTGGCTCACAGAAAAT
SEQ ID NO: 72
GCGACTAAAAGGCACTCTGG
SEQ ID NO: 73
TTGGGTCTCTATGGGGCAGT
SEQ ID NO: 74
TCATCCCATCCATTTTTGGT
SEQ ID NO: 75
ACCCTGTGAAGTGAGGGTTG
SEQ ID NO: 76
GATCCTGCCCTGTCTCTCTG
RARRES2;
SEQ ID NO: 77
CCAATGGGAGGAAACGGAAAT
RARRES2;
SEQ ID NO: 78
CCCATAGAGACCCAAGTTCTG
RARRES2;
SEQ ID NO: 79
CAGGTGGCCCTGGAGGAATTT
Chemerin, amino acid sequence
SEQ ID NO: 80
MRRLLIPLALWLGAVGVGVAELTEAQRRGLQVALEEFHKH
PPVQWAFQETSVESAVDTPFPAGIFVRLEFKLQQTSCRKR
DWKKPECKVRPNGRKRKCLACIKLGSEDKVLGRLVHCPIE
TQVLREAEEHQETQCLRVQRAGEDPHSFYFPGQFAFSKAL
PRS,
SEQ ID NO: 81
CASFWDSW,
SEQ ID NO: 82
CMQHLEYPLTF,

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A method of treating kidney cancer in a subject, comprising administering to the subject a therapeutically effective amount of an inhibitor of chemerin.

2. The method of claim 1, wherein the inhibitor of chemerin is a polypeptide, a polynucleotide, a small molecule, or a gene editing tool.

3. The method of claim 2, wherein the polypeptide is a recombinant antibody.

4. The method of claim 3, wherein the recombinant antibody is a humanized antibody.

5. The method of claim 3, wherein the recombinant antibody comprises a heavy chain variable domain (VH) comprising a heavy chain complementarity-determining region (CDRH)1, a CDRH2, and/or a CDRH3, wherein
the CDRH1 comprises a sequence at least 80% identical to SEQ ID NO: 8 or a fragment thereof,
the CDRH2 comprises a sequence at least 80% identical to SEQ ID NO: 9 or a fragment thereof, and
the CDRH3 comprises a sequence at least 80% identical to SEQ ID NO: 10 or a fragment thereof.

6. The method of claim 5, wherein the VH comprises the sequence of SEQ ID NO: 3 or a fragment thereof.

7. The method of claim 3, wherein the recombinant antibody comprises a light chain variable domain (VL) comprising a light chain complementarity-determining region (CDRL)1, a CDRL2, and/or a CDRL3, wherein
the CDRL1 comprises a sequence at least 80% identical to SEQ ID NO: 14 or a fragment thereof,
the CDRL2 comprises a sequence at least 80% identical to SEQ ID NO: 15 or a fragment thereof, and
the CDRL3 comprises a sequence at least 80% identical to SEQ ID NO: 16 or a fragment thereof.

8. The method of claim 7, wherein the VL comprises the sequence of SEQ ID NO: 4 or a fragment thereof.

9. The method of claim 2, wherein the polynucleotide is an siRNA or an shRNA.

10. The method of claim 2, wherein the gene editing tool is a CRISPR/Cas endonuclease (Cas)9 system.

11. The method of claim 10, wherein the CRISPR/Cas9 system comprises a guide RNA comprising a sequence selected from the group consisting of SEQ ID NOs: 17-40 or a fragment thereof.

12. A method of treating kidney cancer in a subject, comprising:
obtaining a biological sample from the subject;
determining if the biological sample has an increased level of chemerin relative to a reference control; and
administering to the subject a therapeutically effective amount of an anti-cancer agent if the biological sample has an increased level of chemerin.

13. The method of claim 12, wherein the anti-cancer agent comprises an inhibitor of chemerin.

14. A recombinant antibody comprising a heavy chain variable domain (VH) comprising a heavy chain complementarity-determining region (CDRH)1, a CDRH2, and/or a CDRH3, wherein
the CDRH1 comprises a sequence at least 80% identical to SEQ ID NO: 8 or a fragment thereof,
the CDRH2 comprises a sequence at least 80% identical to SEQ ID NO: 9 or a fragment thereof, and
the CDRH3 comprises a sequence at least 80% identical to SEQ ID NO: 10 or a fragment thereof.

15. The recombinant antibody of claim 14, wherein the VH comprises the sequence of SEQ ID NO: 3 or a fragment thereof.

16. The recombinant antibody of claim 14, further comprising a light chain variable domain (VL) comprising a light chain complementarity-determining region (CDRL)1, a CDRL2, and/or a CDRL3, wherein
the CDRL1 comprises a sequence at least 80% identical to SEQ ID NO: 14 or a fragment thereof,
the CDRL2 comprises a sequence at least 80% identical to SEQ ID NO: 15 or a fragment thereof, and
the CDRL3 comprises a sequence at least 80% identical to SEQ ID NO: 16 or a fragment thereof.

17. The recombinant antibody of claim 16, wherein the VL comprises the sequence of SEQ ID NO: 4 or a fragment thereof.

18. The recombinant antibody of claim 14, wherein the recombinant antibody is a humanized antibody.

19. A recombinant polynucleotide comprising a nucleic acid sequence encoding the recombinant antibody of claim 14.

20. An expression vector comprising the recombinant polynucleotide of claim 19.


SEQUENCE LISTING (82 sequences)

SEQ ID NO: 1 - DNA, 396 bp, synthetic construct
  source 1..396: mol_type = other DNA
atggaatggaactgggtcgttctcttcctcctgtcattaactgcaggtgtctattcccagggtcagatgcagcagtctggagctgagctggtgaagcctggggcttcagtgaagctgtcctgcaagacttctggcttcaccttcagcagtagctatataagttggttgaagcagaagcctcgacagagtcttgagtggattgcatggatttatgctggaactggtggtactagctataatcagaagttcacaggcaaggcccaactgactgtagacacatcctccagcacagcctacatgcaactcagcagcctgacatctgaagactctgccatctattactgtgcaagcttctgggactcctggggccaaggcaccactctcacagtctcctca

SEQ ID NO: 2 - DNA, 396 bp, synthetic construct
  source 1..396: mol_type = other DNA
atgaggtgcctagctgagttcctggggctgcttgtgctctggatccctggagccattggggatattgtgatgactcaggctgcaccctctgtacctgtcactcctggagagtcagtatccatctcctgcaggtctagtaagagtctcctgcatagtaatggcaacacttacttgtattggttcctgcagaggccaggccagtctcctcagctcctgatatatcggatgtccaaccttgcctcaggagtcccagacaggttcagtggcagtgggtcaggaactgctttcacactgagaatcagtagagtggaggctgaggatgtgggtgtttattactgtatgcaacatctagaatatccgctcacgttcggtgctgggaccaagctggagctgaaa

SEQ ID NO: 3 - protein, 132 aa, synthetic construct
MEWNWVVLFLLSLTAGVYSQGQMQQSGAELVKPGASVKLSCKTSGFTFSSSYISWLKQKPRQSLEWIAWIYAGTGGTSYNQKFTGKAQLTVDTSSSTAYMQLSSLTSEDSAIYYCASFWDSWGQGTTLTVSS

SEQ ID NO: 4 - protein, 132 aa, synthetic construct
MRCLAEFLGLLVLWIPGAIGDIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPLTFGAGTKLELK

SEQ ID NO: 5 - DNA, 15 bp, synthetic construct
  source 1..15: mol_type = other DNA
agtagctatataagt

SEQ ID NO: 6 - DNA, 51 bp, synthetic construct
  source 1..51: mol_type = other DNA
tggatttatgctggaactggtggtactagctataatcagaagttcacaggc

SEQ ID NO: 7 - DNA, 12 bp, synthetic construct
  source 1..12: mol_type = other DNA
ttctgggactcc

SEQ ID NO: 8 - protein, 5 aa, synthetic construct
SSYIS

SEQ ID NO: 9 - protein, 17 aa, synthetic construct
WIYAGTGGTSYNQKFTG

SEQ ID NO: 10 - protein, 4 aa, synthetic construct
FWDS

SEQ ID NO: 11 - DNA, 48 bp, synthetic construct
  source 1..48: mol_type = other DNA
aggtctagtaagagtctcctgcatagtaatggcaacacttacttgtat

SEQ ID NO: 12 - DNA, 21 bp, synthetic construct
  source 1..21: mol_type = other DNA
cggatgtccaaccttgcctca

SEQ ID NO: 13 - DNA, 27 bp, synthetic construct
  source 1..27: mol_type = other DNA
atgcaacatctagaatatccgctcacg

SEQ ID NO: 14 - protein, 16 aa, synthetic construct
RSSKSLLHSNGNTYLY

SEQ ID NO: 15 - protein, 7 aa, synthetic construct
RMSNLAS

SEQ ID NO: 16 - protein, 9 aa, synthetic construct
MQHLEYPLT

SEQ ID NO: 17 - DNA, 25 bp, synthetic construct
  source 1..25: mol_type = other DNA
caccggaccagtgtggagagcgccg

SEQ ID NO: 18 - DNA, 25 bp, synthetic construct
  source 1..25: mol_type = other DNA
aaaccggcgctctccacactggtcc

SEQ ID NO: 19 - DNA, 25 bp, synthetic construct
  source 1..25: mol_type = other DNA
caccggcgacggctgctgatccctc

SEQ ID NO: 20 - DNA, 25 bp, synthetic construct
  source 1..25: mol_type = other DNA
aaacgagggatcagcagccgtcgcc

SEQ ID NO: 21 - DNA, 25 bp, synthetic construct
  source 1..25: mol_type = other DNA
caccgctatggggcagtggaccaac

SEQ ID NO: 22 - DNA, 25 bp, synthetic construct
  source 1..25: mol_type = other DNA
aaacgttggtccactgccccatagc

SEQ ID NO: 23 - DNA, 25 bp, synthetic construct
  source 1..25: mol_type = other DNA
caccgccagtgctggcttagctgcg

SEQ ID NO: 24 - DNA, 25 bp, synthetic construct
  source 1..25: mol_type = other DNA
aaaccgcagctaagccagcactggc

SEQ ID NO: 25 - DNA, 25 bp, synthetic construct
  source 1..25: mol_type = other DNA
caccgcccttcttacccgcagaact

SEQ ID NO: 26 - DNA, 25 bp, synthetic construct
  source 1..25: mol_type = other DNA
aaacagttctgcgggtaagaagggc

SEQ ID NO: 27 - DNA, 25 bp, synthetic construct
  source 1..25: mol_type = other DNA
caccgattgggcctgactttgcact

SEQ ID NO: 28 - DNA, 25 bp, synthetic construct
  source 1..25: mol_type = other DNA
aaacagtgcaaagtcaggcccaatc

SEQ ID NO: 29 - DNA, 25 bp, synthetic construct
  source 1..25: mol_type = other DNA
caccggaaccaccgcagcgttcgcc

SEQ ID NO: 30 - DNA, 25 bp, synthetic construct
  source 1..25: mol_type = other DNA
aaacggcgaacgctgcggtggttcc

SEQ ID NO: 31 - DNA, 25 bp, synthetic construct
  source 1..25: mol_type = other DNA
caccgcaaactgcagcgcaaccgcc

SEQ ID NO: 32 - DNA, 25 bp, synthetic construct
  source 1..25: mol_type = other DNA
aaacggcggttgcgctgcagtttgc

SEQ ID NO: 33 - DNA, 25 bp, synthetic construct
  source 1..25: mol_type = other DNA
caccgtgtggggtatagccggcaca

SEQ ID NO: 34 - DNA, 25 bp, synthetic construct
  source 1..25: mol_type = other DNA
aaactgtgccggctataccccacac

SEQ ID NO: 35 - DNA, 25 bp, synthetic construct
  source 1..25: mol_type = other DNA
caccgccatatcacctatgccgcca

SEQ ID NO: 36 - DNA, 25 bp, synthetic construct
  source 1..25: mol_type = other DNA
aaactggcggcataggtgatatggc

SEQ ID NO: 37 - DNA, 25 bp, synthetic construct
  source 1..25: mol_type = other DNA
caccggtattcatcaccgtaactga

SEQ ID NO: 38 - DNA, 25 bp, synthetic construct
  source 1..25: mol_type = other DNA
aaactcagttacggtgatgaatacc

SEQ ID NO: 39 - DNA, 25 bp, synthetic construct
  source 1..25: mol_type = other DNA
caccggcgctgcagtttgcacacga

SEQ ID NO: 40 - DNA, 25 bp, synthetic construct
  source 1..25: mol_type = other DNA
aaactcgtgtgcaaactgcagcgcc

SEQ ID NO: 41 - RNA, 19 nt, synthetic construct
  source 1..19: mol_type = other RNA
gcctagagctggaacgtta

SEQ ID NO: 42 - RNA, 19 nt, synthetic construct
  source 1..19: mol_type = other RNA
gcaggaaagtttacaccaa

SEQ ID NO: 43 - RNA, 19 nt, synthetic construct
  source 1..19: mol_type = other RNA
tgcaagaagtgatgagtta

SEQ ID NO: 44 - RNA, 19 nt, synthetic construct
  source 1..19: mol_type = other RNA
aaattgagctcctctgtca

SEQ ID NO: 45 - DNA, 20 bp, synthetic construct
  source 1..20: mol_type = other DNA
tcggagatgggtttaaggtg

SEQ ID NO: 46 - DNA, 20 bp, synthetic construct
  source 1..20: mol_type = other DNA
ccgaaaacatcctcacaggt

SEQ ID NO: 47 - DNA, 20 bp, synthetic construct
  source 1..20: mol_type = other DNA
ggtcgcttttgggattacct

SEQ ID NO: 48 - DNA, 20 bp, synthetic construct
  source 1..20: mol_type = other DNA
catgtacgttgctatccagg

SEQ ID NO: 49 - DNA, 20 bp, synthetic construct
  source 1..20: mol_type = other DNA
gaagatggcagaagctcacc

SEQ ID NO: 50 - DNA, 20 bp, synthetic construct
  source 1..20: mol_type = other DNA
tggccactacaaacaagcaa

SEQ ID NO: 51 - DNA, 25 bp, synthetic construct
  source 1..25: mol_type = other DNA
gacatgaagttcacctactgtgatg

SEQ ID NO: 52 - DNA, 20 bp, synthetic construct
  source 1..20: mol_type = other DNA
ccagctgggagaagagtttg

SEQ ID NO: 53 - DNA, 20 bp, synthetic construct
  source 1..20: mol_type = other DNA
cagctatttgcgtgtgagga

SEQ ID NO: 54 - DNA, 20 bp, synthetic construct
  source 1..20: mol_type = other DNA
cagagactcgagcacagcac

SEQ ID NO: 55 - DNA, 20 bp, synthetic construct
  source 1..20: mol_type = other DNA
ggactgagaaaggggaaagg

SEQ ID NO: 56 - DNA, 20 bp, synthetic construct
  source 1..20: mol_type = other DNA
cagatgcaggaagcatcaaa

SEQ ID NO: 57 - DNA, 20 bp, synthetic construct
  source 1..20: mol_type = other DNA
gtgcaaagtcaggcccaatg

SEQ ID NO: 58 - DNA, 20 bp, synthetic construct
  source 1..20: mol_type = other DNA
cagtgagagcccagaaggac

SEQ ID NO: 59 - DNA, 20 bp, synthetic construct
  source 1..20: mol_type = other DNA
gagggctgcatctctaccag

SEQ ID NO: 60 - DNA, 20 bp, synthetic construct
  source 1..20: mol_type = other DNA
tcctcacaccattgaaacca

SEQ ID NO: 61 - DNA, 20 bp, synthetic construct
  source 1..20: mol_type = other DNA
cgtaagccttctgagccatc

SEQ ID NO: 62 - DNA, 20 bp, synthetic construct
  source 1..20: mol_type = other DNA
gttcctccacttggtctcca

SEQ ID NO: 63 - DNA, 20 bp, synthetic construct
  source 1..20: mol_type = other DNA
tccagttccgatttgtaggc

SEQ ID NO: 64 - DNA, 21 bp, synthetic construct
  source 1..21: mol_type = other DNA
ctccttaatgtcacgcacgat

SEQ ID NO: 65 - DNA, 19 bp, synthetic construct
  source 1..19: mol_type = other DNA
tggcgtacatcgttgtcat

SEQ ID NO: 66 - DNA, 20 bp, synthetic construct
  source 1..20: mol_type = other DNA
tggcacagtaaccaaatcca

SEQ ID NO: 67 - DNA, 19 bp, synthetic construct
  source 1..19: mol_type = other DNA
cggagtctagcgcatggta

SEQ ID NO: 68 - DNA, 20 bp, synthetic construct
  source 1..20: mol_type = other DNA
ctcatagtggcgaacagcaa

SEQ ID NO: 69 - DNA, 23 bp, synthetic construct
  source 1..23: mol_type = other DNA
ttcatctgtgctttcatgtcatc

SEQ ID NO: 70 - DNA, 20 bp, synthetic construct
  source 1..20: mol_type = other DNA
gatgaccggggtttaaaggt

SEQ ID NO: 71 - DNA, 20 bp, synthetic construct
  source 1..20: mol_type = other DNA
ggacgtggctcacagaaaat

SEQ ID NO: 72 - DNA, 20 bp, synthetic construct
  source 1..20: mol_type = other DNA
gcgactaaaaggcactctgg

SEQ ID NO: 73 - DNA, 20 bp, synthetic construct
  source 1..20: mol_type = other DNA
ttgggtctctatggggcagt

SEQ ID NO: 74 - DNA, 20 bp, synthetic construct
  source 1..20: mol_type = other DNA
tcatcccatccatttttggt

SEQ ID NO: 75 - DNA, 20 bp, synthetic construct
  source 1..20: mol_type = other DNA
accctgtgaagtgagggttg

SEQ ID NO: 76 - DNA, 20 bp, synthetic construct
  source 1..20: mol_type = other DNA
gatcctgccctgtctctctg

SEQ ID NO: 77 - DNA, 21 bp, synthetic construct
  source 1..21: mol_type = other DNA
ccaatgggaggaaacggaaat

SEQ ID NO: 78 - DNA, 21 bp, synthetic construct
  source 1..21: mol_type = other DNA
cccatagagacccaagttctg

SEQ ID NO: 79 - DNA, 21 bp, synthetic construct
  source 1..21: mol_type = other DNA
caggtggccctggaggaattt

SEQ ID NO: 80 - protein, 163 aa, synthetic construct
MRRLLIPLALWLGAVGVGVAELTEAQRRGLQVALEEFHKHPPVQWAFQETSVESAVDTPFPAGIFVRLEFKLQQTSCRKRDWKKPECKVRPNGRKRKCLACIKLGSEDKVLGRLVHCPIETQVLREAEEHQETQCLRVQRAGEDPHSFYFPGQFAFSKALPRS

SEQ ID NO: 81 - protein, 8 aa, synthetic construct
CASFWDSW

SEQ ID NO: 82 - protein, 11 aa, synthetic construct
CMQHLEYPLTF